SEQUENCE LISTING (118 sequences)

SEQ ID NO: 1 - protein, 341 aa, Mycobacterium tuberculosis
  misc_feature (341)..(341): Xaa can be any naturally occurring amino acid
MTFFEQVRRLRSAATTLPRRLAIAAMGAVLVYGLVGTFGGPATAGAFSRPGLPVEYLQVPSASMGRDIKVQFQGGGPHAVYLLDGLRAQDDYNGWDINTPAFEEYYQSGLSVIMPVGGQSSFYTDWYQPSQSNGQNYTYKWETFLTREMPAWLQANKGVSPTGNAAVGLSMSGGSALILAAYYPQQFPYAASLSGFLNPSEGWWPTLIGLAMNDSGGYNANSMWGPSSDPAWKRNDPMVQIPRLVANNTRIWVYCGNGTPSDLGGDNIPAKFLEGLTLRTNQTFRDTYAADGGRNGVFNFPPNGTHSWPYWNEQLVAMKADIQHVLNGATPPAAPAAPAAX

SEQ ID NO: 2 - protein, 341 aa, Mycobacterium tuberculosis
  misc_feature (341)..(341): Xaa can be any naturally occurring amino acid
MNAPKLVIGANGFLGSHVTRQLVADCAPQKGEVRAMVRPAANTRSIDDLPLTRFHGDVFDTATVAEAMAGCDDVYYCVVDTRAWLRDPSPLFRTNVAGLRNVLDVATDASLRRFVFTSSYATVGRRRGHVATEEDRVDTRKVTPYVRSRVAAEDLVLQYAHDAGLPAVAMCVSTTYGGGDWGRTPHGAFIAGAVFGRLPFTMRGIRLEAVGVDDAARALILAAERGRNGERYLISERMMPLQEVVRIAADEAGVPPPRWSISVPVLYALGALGSLRARLTGKDTELSLASVRMMRSEADVDHGKAVRELGWQPRPVEESIREAARFWAAMRTVGKDPAASX

SEQ ID NO: 3 - protein, 323 aa, Mycobacterium tuberculosis
  misc_feature (323)..(323): Xaa can be any naturally occurring amino acid
MTVVVEKTPTTLPQATPNGAAPWHVRAGAFAIDVLPGLAVAATMALTALTVPPGSAWRWLCACLLGLTILLLAVNRLLLPTITGWSLGRALTGIRVVRRDGSAIGPWRLLVRDLAHLVDTLSLFVGWLWPLWDSRRRTFADLLLRTEVRRVEPVQRPAVIRRLTAAVALAAAGACASATAVGAAVVYVNEWQTDHTRAQLATRGPKLVVDVLSYDPETVQRDFERARSLATDRYRPQLSIQQDSVRESGPVRNQYWVTDSAVLSATPAQATMLLFMQGERGTPPNQRYIQSTVRAIFQKSRGQWRLDDLAVVMKPRQPTGEKX

SEQ ID NO: 4 - protein, 453 aa, Mycobacterium tuberculosis
  misc_feature (453)..(453): Xaa can be any naturally occurring amino acid
MSQAQPRPAAPNPKRNVKAIRTVRFWMAPIATTLALMSALAALYLGGILNPMTNLRHFPIALVNEDAGPAGQQIVDGLVSGLDKNKFDIRVVSPDEARRLLDTAAVYGSALIPPTFSSQLRDFGASAVTPTRTDRPAITISTNPRAGTLAASIAGQTLTRALTVVNGKVGERLTAEVAAQTGGVALAGAAAAGLASPIDVKSTAYNPLPNGTGNGLSAFYYALLLLLAGFTGSIVVSTLVDSMLGYVPAEFGPVYRFAEQVNISRFRTLLVKWAVMVVLALLTSGVYLAIAHGLGMPIPLGWQVWLYGVFAIIAVGVTSSSLIAVLGSMGLLVSMLIFVILGLPSAGATVPLEAVPAFFRWLAQFEPMHQVFLGVRSLLYLNGNADAGLSQALTMTSIGLIIGLLLGGFITHLYDRSSFHRIPGAVEMAIAVEHQAQYQARQSARESSSEQPX

SEQ ID NO: 5 - protein, 692 aa, Mycobacterium tuberculosis
  misc_feature (692)..(692): Xaa can be any naturally occurring amino acid
MTDDERFSLLVGLTGASDLWPVRDERIPQGVPMCAGYVPGIPRLGVPALLMSDAGLGVTNPGYRPGDTATALPAGLALAASFNPVLARSSGKAIGREARSRGFNVQLAGAINLARDPRNGRNFEYLSEDPLLSATMAAESIIGIQQQGVIATTKHFSLNCNETNRHWLDAVIDPDAHRESDLLAFEIVIERSQPGAVMAAYNKVNGDYAAGNDHLLNDVLKGAWGYRGWVMSDWGGTPSWECALAGLDQECGAQIDAVLWQSEAFTDRLRAAYADGNLPKGRLSDMVRRILRSMFAVGIDRWKPAPAPDMNAHNEIAAQMARQGIVLLQNRGLLPLAPESAGRIAVIGGYAHLGVPAGYGSSAVTPPGGYAGVIPIGGSGLAAGLRNLYLLPSSPLSELRKRLPNAQFEFDPGINPAEAVLAARRADIAIVFAIRAEGEGFDSADLSLPWGQDALIAAVASANANTVVVLETGNPVTMPWRDSVNAIMQAWYPGQAGGQAVAEIVTGQVNPSGRLPITFPVDLGQTPRSQPPELGAPWGTSTTIHYTEGADVGYRWFASTNQTPMFAFGHGLSYTSFEYRDLVVTGGHTVHASFSVTNTGDRSGADVPQLYMIAAPGESRLRLLGFERVELEPGQTRRVRIEADPRLLARYDGEARSWRIEPGGYTVAVGASAVALKLAAKVKLAGRGFGRX

SEQ ID NO: 6 - protein, 511 aa, Mycobacterium tuberculosis
MRSVTCSATLVLPVIEPTPADRRPRHLLLGSAGHVSGRLDTGRFVQTHPAKDVSVPIATINPATGETVKTFTAATDDEVDAAIARAHRRFADYRQTSFAQRARWANATADLLEAEADQAAAMMTLEMGKTLAAAKAEALKCAKGFRYYAENAEALLADEPADAAKVGASAAYGRYQPLGVILAVMPWNFPLWQAVRFAAPALMAGNVGLLKHASNVPQCALYLADVIARGGFPDGCFQTLLVSSGAVEAILRDPRVAAATLTGSEPAGQSVGAIAGNEIKPTVLELGGSDPFIVMPSADLDAAVSTAVTGRVQNNGQSCIAAKRFIVHADIYDDFVDKFVARMAALRVGDPTDPDTDVGPLATEQGRNEVAKQVEDAAAAGAVIRCGGKRLDRPGWFYPPTVITDISKDMALYTEEVFGPVASVFRAANIDEAVEIANATTFGLGSNAWTRDETEQRRFIDDIVAGQVFINGMTVSYPELPFGGVKRSGYGRELSAHGIREFCNIKTVWIA

SEQ ID NO: 7 - protein, 160 aa, Mycobacterium tuberculosis
  misc_feature (160)..(160): Xaa can be any naturally occurring amino acid
MNNLALWSRPVWDVEPWDRWLRDFFGPAATTDWYRPVAGDFTPAAEIVKDGDDAVVRLELPGIDVDKDVNVELDPGQPVSRLVIRGEHRDEHTQDAGDKDGRTLREIRYGSFRRSFRLPAHVTSEAIAASYDAGVLTVRVAGAYKAPAETQAQRIAITKX

SEQ ID NO: 8 - protein, 285 aa, Mycobacterium tuberculosis
  misc_feature (285)..(285): Xaa can be any naturally occurring amino acid
MVPLWFTLSALCFVGAVVLLYVDIDRRRGRSRRRKSWARSHGFDYERESTEILKRWTRGVMSTVGDVAAHNVVLGQIRGEAVYIFDLEEVATVIALHRKVGTNVVVDLRLKGLKEPRESDIWLLGAIGPRMVYSTNLDAARRACDRRMVTFAHTAPDCAEIMWNEQNWTLVSMPIASTRAQWDEGLRTVRQFNDLLRVLPPLPQEMPQQTGVGPRGAAPGRPVAPGGPAELPPRRAQPDPATTVLPDPARRAPEPIRRDEGRSEGVRRPPPAGRNGQQATNYQHX

SEQ ID NO: 9 - protein, 386 aa, Mycobacterium tuberculosis
  misc_feature (386)..(386): Xaa can be any naturally occurring amino acid
MSSTVLVINSGSSSLKFQLVEPVAGMSRAAGIVERIGERSSPVADHAQALHRAFKMLAEDGIDLQTCGLVAVGHRVVHGGTEFHQPTLLDDTVIGKLEELSALAPLHNPPAVLGIKVARRLLANVAHVAVFDTAFFHDLPPAAATYAIDRDVADRWHIRRYGFHGTSHQYVSERAAAFLGRPLDGLNQIVLHLGNGASASAIARGRPVETSMGLTPLEGLVMGTRSGDLDPGVISYLWRTARMGVEDIESMLNHRSGMLGLAGERDFRRLRLVIETGDRSAQLAYEVFIHRLRKYLGAYLAVLGHTDVVSFTAGIGENDAAVRRDALAGLQGLGIALDQDRNLGPGHGARRISSDDSPIAVLVVPTNEELAIARDCLRVLGGRRAX

SEQ ID NO: 10 - protein, 329 aa, Mycobacterium tuberculosis
  misc_feature (329)..(329): Xaa can be any naturally occurring amino acid
MVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPX

SEQ ID NO: 11 - protein, 129 aa, Mycobacterium tuberculosis
  misc_feature (129)..(129): Xaa can be any naturally occurring amino acid
MEILASRMLLRPADYQRSLSFYRDQIGLAIAREYGAGTVFFAGQSLLELAGYGEPDHSRGPFPGALWLQVRDLEATQTELVSRGVSIAREPRREPWGLHEMHVTDPDGITLIFVEVPEGHPLRTDTRAX

SEQ ID NO: 12 - protein, 435 aa, Mycobacterium tuberculosis
  misc_feature (435)..(435): Xaa can be any naturally occurring amino acid
MLEVAAEPTRRRLLQLLAPGERTVTQLASQFTVTRSAISQHLGMLAEAGLVTARKQGRERYYRLDERGVLRLRALMESFWSDELDRLVADAAHYPPSQGDCAMPFEKAVVVPLDPTSTFALITQPDRLRRWMAVAARIELRTGGAYRWTVTPGHSAAGTVIDVDPGKRVVFTWGWEDHGDPPPGGSTVTITLTPVDGGTEVRLVHDGLTAQQAARHAKGWNHFLDRLVVAGQRGDAGPDEWAAAPDPLDELSCAEATLAVLQHVLRGIGASDLTRQTPCTEYDVSQLADHLLRSLAIIGAAAGAQLAPRDVDAPLETQVADAAQAVMEAWRRRGLAGTVELNSNQVPATVPVGILCLEFLVHAWDFAIATGSQVIASEPVSEYVLAVAGKVITPATRNSAGFAAPAAVGSFAPVLDRLIAFTGRQPTAGHVSATX

SEQ ID NO: 13 - protein, 162 aa, Mycobacterium tuberculosis
  misc_feature (162)..(162): Xaa can be any naturally occurring amino acid
MSDEGDVADEAVADGAENADSRGSGGRTALVTKPVVRPQRPTGKRSRSRAAGADADVDVEEPSTAASEATGVAKDDSTTKAVSKAARAKKASKPKARSVNPIAFVYNYLKQVVAEMRKVIWPNRKQMLTYTSVVLAFLAFMVALVAGADLGLTKLVMLVFGX

SEQ ID NO: 14 - protein, 336 aa, Mycobacterium tuberculosis
  misc_feature (336)..(336): Xaa can be any naturally occurring amino acid
MAVPAVSPQPILAPLTPAAIFLVATIGADGEATVHDALSKISGLVRAIGFRDPTKHLSVVVSIGSDAWDRLFAGPRPTELHPFVELTGPRHTAPATPGDLLFHIRAETMDVCFELAGRILKSMGDAVTVVDEVHGFRFFDNRDLLGFVDGTENPSGPIAIKATTIGDEDRNFAGSCYVHVQKYVHDMASWESLSVTEQERVIGRTKLDDIELDDNAKPANSHVALNVITDDDGTERKIVRHNMPFGEVGKGEYGTYFIGYSRTPTVTEQMLRNMFLGDPAGNTDRVLDFSTAVTGGLFFSPTIDFLDHPPPLPQAATPTLAAGSLSIGSLKGSPRX

SEQ ID NO: 15 - protein, 158 aa, Mycobacterium tuberculosis
  misc_feature (158)..(158): Xaa can be any naturally occurring amino acid
MIANLVAVAIRASREVVIEAPPEVIVEALADMDAVPSWSSVHKRVEVVDTYSDGRPHHVKVTIKVAGIVDTELLEYHWGPDWVVWDAAKTAQQHGQHGEYNLRREDNDKTRVRFTLTVEPSAPLPAFWVNIARKKILHAATEGLRKQVVGRRRFTSGX

SEQ ID NO: 16 - protein, 607 aa, Mycobacterium tuberculosis
  misc_feature (607)..(607): Xaa can be any naturally occurring amino acid
MSYVLATPEMVAAAANNLAQIGSTLSAANAAALAPTTGVLAAGADEVSAAVASLFSGHAQAYQTLGTQAAAFHERFIQALSTAAGAYGSAEAANASPLQQALNVINAPTQTLLGRPLIGNGTNGAPGTGQAGGPGGLLYGNGGNGGSGGVGQAGGAGGSAGLIGIGGTGGAGGAGAVGGVGGNGGWLYGNGGAGGLGGTGVAGVNGGMGAAGGAGGNAYLFGSGGAGGQGGMGAAGADGVNPTPTGTADAGSTGTDQTLGGNAIGGNGGPGDAGDAMTSGGAGGSGGNAVSTVNGDAVGGEGGKGGEGAYGGAGGAGGSAASIGNAAIGGNGGAGGNAQAPGGVGGAGGEGGDAQVGTNSPSNAEAGNGGSGGNGFDSFASGGTGGAGGTGGAGGRGGLLIGDGGAGGAGGVGGTGGSGAPGGGGGAGGDGGAANTDSAGSSRKAFGGDGGVGGDGASALGTGGEGGIGGQGGNGGAGGLLIGNGGAGGVGGTAGAGGTGGSGGAGGAGGAGGGGTNSGPGAAFGGNGNTGGNGGNGGAPGALGGKGGSGGLIGRAGSDGGVGAGGAGGAGGAGGTGGEGGTGGDGKTTDGNPGMGGSPGSAGQPGX

SEQ ID NO: 17 - protein, 536 aa, Mycobacterium tuberculosis
  misc_feature (536)..(536): Xaa can be any naturally occurring amino acid
MSDHDRDFDVVVVGGGHNGLVAAAYLARAGLRVRLLERLAQTGGAAVSIQAFDGVEVALSRYSYLVSLLPSRIVADLGAPVRLARRPFSSYTPAPATAGRSGLLIGPTGEPRAAHLAAIGAAPDAHGFAAFYRRCRLVTARLWPTLIEPLRTREQARRDIVEYGGHEAAAAWQAMVDEPIGHAIAGAVANDLLRGVIATDALIGTFARMHEPSLMQNICFLYHLVGGGTGVWHVPIGGMGSVTSALATAAARHGAEIVTGADVFALDPDGTVRYHSDGSDGAEHLVRGRFVLVGVTPAVLASLLGEPVAALAPGAQVKVNMVVRRLPRLRDDSVTPQQAFAGTFHVNETWSQLDAAYSQAASGRLPDPLPCEAYCHSLTDPSILSARLRDAGAQTLTVFGLHTPHSVFGDTEGLAERLTAAVLASLNSVLAEPIQDVLWTDAQSKPCIETTTTLDLQRTLGMTGGNIFHGALSWPFADNDDPLDTPARQWGVATDHERIMLCGSGARRGGAVSGIGGHNAAMAVLACLASRRKSPX

SEQ ID NO: 18 - protein, 383 aa, Mycobacterium tuberculosis
  misc_feature (383)..(383): Xaa can be any naturally occurring amino acid
MNIWTTPERQQLRKTVRAFAEREILPHVDEWERIGELPRGLHRLAGAAGLLGAGFPEAVGGGGGDGADPVIICEEMHQAGAPGGVYASLFTCGIAVPHMVASGDERLIATYVRPTLAGEKIGALAITEPGGGSDVGHLRTSAVRDGDHYVINGAKTYITSGVRADYVVTAVRTGGPGAAGVSLLVVEKDTPGFEVTRKLDKMGWRSSDTAELCYTDVAVPATNLVGAENSGFTQIARAFVSERIGLAAQAYSSAQRCLDLTAQWCRDRETFGRPLISRQSVQNTLAEMARRIDVARVYAHHVVERQLAGETDLIAQVCFAKNTAVQAGEWVANQAVQLFGGMGYMAESEVERQYRDMRILGIGGGTTEILTALAAKTLGYQSX

SEQ ID NO: 19 - protein, 254 aa, Mycobacterium tuberculosis
MSVDYPQMAATRGRIEPAPRRVRGYLGHVLVFDTSAARYVWEVPYYPQYYIPLADVRMEFLRDENHPQRVQLGPSRLHSLVSAGQTHRSAARVFDVDGDSPVAGTVRFNWDPLRWFEEDEPIYGHPRNPYQRADALRSHRHVRVELDGIVLADTRSPVLLFETGIPTRYYIDPADIAFEHLEPTSTQTLCPYKGTTSGYWSVRVGDAVHRDLAWTYHYPLPAVAPIAGLVAFYNEKVDLTVDGVALPRPHTQFS

SEQ ID NO: 20 - protein, 768 aa, Mycobacterium tuberculosis
  misc_feature (768)..(768): Xaa can be any naturally occurring amino acid
MSFVVVAPEVLAAAASDLAGIGSTLAQANAAALAPTTAVLAAGADEVSAAIASLFGAHGQAYQAVSAQMSAFHAQFMQALTGAGGAYAAAEAVNVSAAQSVEQDLLAAINARFERIFGRPLIGDGANGGPGQDGGPGGLLYGNGGNGGTSTTVGMAGGNGGAAGLIGNGGFGGGGGPGAAGGNGGAGGWLFGNGGAGGAGGLGVAPGVPGGAGGAGGAGGVGGPAGLWGHGGAGGAGGAGVAGAGGFEGTIGAGGAGGVGGAGGVGGAGGAGGWLYGDAGAGGDGGVGGAGGTGGLGNRGGAGGAGGAGGVGGAGGAAGLWGGGGAGGVGGTGGGAGLGAQSVTFSSSLSGLSGGDGGAGGAGGAGGAGGTGGWLYGGGGAAGSGGDGGTGGQGGAGGAGVFSLFGSGGGPGGNGGVGGVGGVGGAGGRAGLFGVGGLGGAGGDAGDSGEGGFGGPGLAGGLFGNPGNGGVGGIGGDAAAGGAGGAGGNGGAGGNGGWLFGNGGAGGSGGDGGAAGRGGAGNLGSAGGINAPAGNPGSGSVGIGGAGGAGGTAGLFGDGGAGGAGGAGAAGGFGGISAATPSAGSEGAMGGAGGVGGNARLLGTGGAGGVGGGGGAGGDGGRGGVATPGGQGGDAGDGGAGGAGGNGGGASGAGGWLLGTGGAGGAGGNGGNGGKAGFSPGPTNFGLNGAGGGGGVGGNGATGPWLFGDGGPTPGSTGAGAAGGHGGDAQLIGNGGHGGAGGTGVPNGSGGAGGLSGLLFGEPGANGX

SEQ ID NO: 21 - protein, 854 aa, Mycobacterium tuberculosis
  misc_feature (854)..(854): Xaa can be any naturally occurring amino acid
MSFVIAAPEALVAVASDLAGIGSALAEANAAALAPTTALLAAGADEVSAAIAALFGAHGQAYQTVSAQASAFHAQFVQALTGGGGAYAAAEAANVSAAQSTDQRLLDLINGPTQALLGRPLIGDGANGGPGQDGGPGGLLYGNGGNGGTSTTAGVAGGNGGAAGLIGNGGAGGGGGAGAAGGNGGAGGWLYGNGGAGGAGGTSVIPGVAGGNGGAGGSAGLWGTGGAGGDGGNGRSGPVNVAGSAGGNGGAGGAAGLFGDAGAGGNGGKGGAGGAAFSINFTAGDGGAGGAGGSGGHALLWGAGGAGGNGGSGGTGGAGGSTAGAGGNGGAGGGGGTGGLLFGNGGAGGHGAAAGNGLAAGNGVSSSGGGGAGGTGGAGGDGGAGGAGGNARLWGVGGAGGAGGDGGAGGAGGKGGSGLSGNANGGAGGDSGRGGTGGAGGEGGAAGLLVGTGGHGGDGGAGGAAVKGGDGGAAAGTGIAGAGGRGGAGGSGGSGGDGGGGAAGPAGWLFGDGGAGGNGGAAAAGGAGGQAGGGGGNGGNGGNGGNGGNGGNGATGGWLYGNGGAGGQGATAGAGGAGANGVSSTNGGGTGGNGGIGGTGGSGGAGGNAGLLGVGGAGGHGASGGAGDRGGAGGTGFISSDGGAGGDGGDGGNGGAGGTGGLLFGAGGNGGPGGSGGAADIGGNGGAGNGGGTDGNGGNGGSGGGAGSGGDGGGAGGNGAWLFGNGGAGGGGGKGGNGAGGGLGGGSFGLPGLNGSGGDGGDGGNGAPGGVLYGNGGAGGQGSSGGIGGPGATGGAGGKGGDGGDAQLIGDGGNGGNGGAGGTGGTPGPGGPGGSGGLGGLLFGQTGTAGVSPX

SEQ ID NO: 22 - protein, 857 aa, Mycobacterium tuberculosis
  misc_feature (857)..(857): Xaa can be any naturally occurring amino acid
MPQPVGRKSTALPSPVVPPQANASALRRVLRRARDGVTLNVDEAAIAMTARGDELADLCASAARVRDAGLVSAGRHGPSGRLAISYSRKVFIPVTRLCRDNCHYCTFVTVPGKLRAQGSSTYMEPDEILDVARRGAEFGCKEALFTLGDRPEARWRQAREWLGERGYDSTLSYVRAMAIRVLEQTGLLPHLNPGVMSWSEMSRLKPVAPSMGMMLETTSRRLFETKGLAHYGSPDKDPAVRLRVLTDAGRLSIPFTTGLLVGIGETLSERADTLHAIRKSHKEFGHIQEVIVQNFRAKEHTAMAAFPDAGIEDYLATVAVARLVLGPGMRIQAPPNLVSGDECRALVGAGVDDWGGVSPLTPDHVNPERPWPALDELAAVTAEAGYDMVQRLTAQPKYVQAGAAWIDPRVRGHVVALADPATGLARDVNPVGMPWQEPDDVASWGRVDLGAAIDTQGRNTAVRSDLASAFGDWESIREQVHELAVRAPERIDTDVLAALRSAERAPAGCTDGEYLALATADGPALEAVAALADSLRRDVVGDEVTFVVNRNINFTNICYTGCRFCAFAQRKGDADAYSLSVGEVADRAWEAHVAGATEVCMQGGIDPELPVTGYADLVRAVKARVPSMHVHAFSPMEIANGVTKSGLSIREWLIGLREAGLDTIPGTAAEILDDEVRWVLTKGKLPTSLWIEIVTTAHEVGLRSSSTMMYGHVDSPRHWVAHLNVLRDIQDRTGGFTEFVPLPFVHQNSPLYLAGAARPGPSHRDNRAVHALARIMLHGRISHIQTSWVKLGVRRTQVMLEGGANDLGGTLMEETISRMAGSEHGSAKTVAELVAIAEGIGRPARQRTTTYALLAAX

SEQ ID NO: 23 - protein, 330 aa, Mycobacterium tuberculosis
  misc_feature (330)..(330): Xaa can be any naturally occurring amino acid
MAGWFAHTLRPAMLAAGRSDRLGRIVERSPLTRGVVRRFVPGDTLDDVVDIVTALRDSGRYLSIDYLGENVTDADDAAAAVRAYLGLLDVLGRRGDIACDGVRPLEVSLKLSALGQALDRDGQKIALDNARAICERAERVGAWVTVDAEDHTTTDSTLSISGDLRVDFPWLGTVVQAYLRRTLADCAELAAVGARVRLCKGAYDEPASVAYRDAAQVTDSYLRCLRVLTAGRGYPMVATHDPVIIAAVPGITRESGRSQGDFEYQMLYGVRDDEQRRLTGAGNHVRVYVPFGTRWYGYFLRRLAERPANLAFFLRALTDRRRARGCAERX

SEQ ID NO: 24 - protein, 562 aa, Mycobacterium tuberculosis
  misc_feature (562)..(562): Xaa can be any naturally occurring amino acid
MARNPSPALDRPWRRPGALRYALERVRGVAKPPITVTDPPADVVIERDVEVPTRDGTLLRINVFRSAEGGARPVIASIHPYGKDALPRRRGNRWTFSPQYRMLRQPKPLTFSALTGWEAPDPAWWTAQGFVVVNADSRGCGRSDGTGDLLSHQEAEDTYDLVGWLADQAWSDGRVVMLGVSYLAISQYAVAALQPPALRAICPWEGFTDAYRDLAFPGGIRESGFTRLWSRGVRRRTRQTYDMEQMQEAHPLRDDFWRSRVPDLSAIKVPMLVCGSFSDNNLHSRGSIRAFTRSGCGHARLYTHRGGKWETFYSATALSEQLKFLRDALAGSSGSRSVRLEVREDRDTITAVREETQWPLAGTRWRPMYLAGPGLLATEPPPTAGSIRFQTRSRAAAFNWTIPEDIELTGPMAARLWVQLDGCDDANLFVGVEKWRDGQFVAFEGSYGWGRDRVTTGWQRVSLRELDPELSQPWEPVPACARPRPVTAGEVVAVDVALGPSATLFRAGEQLRLVVGGRWLSPRNPLTGQFPAAYPRPPRGRVTLHWGPRYDAHLLIPEVPGX

SEQ ID NO: 25 - protein, 373 aa, Mycobacterium tuberculosis
  misc_feature (373)..(373): Xaa can be any naturally occurring amino acid
MKTVVVSGASVAGTAAAYWLGRHGYSVTMVERHPGLRPGGQAIDVRGPALDVLERMGLLAAAQEHKTRIRGASFVDRDGNELFRDTESTPTGGPVNSPDIELLRDDLVELLYGATQPSVEYLFDDSISTLQDDGDSVRVTFERAAAREFDLVIGADGLHSNVRRLVFGPEEQFVKRLGTHAAIFTVPNFLELDYWQTWHYGDSTMAGVYSARNNTEARAALAFMDTELRIDYRDTEAQFAELQRRMAEDGWVRAQLLHYMRSAPDFYFDEMSQILMDRWSRGRVALVGDAGYCCSPLSGQGTSVALLGAYILAGELKAAGDDYQLGFANYHAEFHGFVERNQWLVSDNIPGGAPIPQEEFERIVHSITIKDYX

SEQ ID NO: 26 - protein, 702 aa, Mycobacterium tuberculosis
  misc_feature (702)..(702): Xaa can be any naturally occurring amino acid
MSGRAIGTETEWWVPGRVEIDDVAPVVSCGVYPAKAVVGEVVPVSAAVWREGHEAVAATLVVRYLGVRYPHLTDRPRARVLPTPSEPQQRVKPLLIPMTSGQEPFVFHGQFTPDRVGLWTFRVDGWGDPIHTWRHGLIAKLDAGQGETELSNDLLVGAVLLERAATGVPRGLRDPLLAAAAALRTPGDPVTRTALALTPEIEELLADYPLRDLVTRGEQFGVWVDRPLARFGAWYEMFPRSTGGWDDDGNPVHGTFATAAAELPRIAGMGFDVVYLPPIHPIGKVHRKGRNNSPTAAPTDVGSPWAIGSDEGGHDTVHPSLGTIDDFDDFVSAARDLGMEVALDLALQCAPDHPWAREHRQWFTELPDGTIAYAENPPKKYQDIYPLNFDNDPEGLYDEVLRVVQHWVNHGVKFFRVDNPHTKPPNFWAWLIAQVKTVDPDVLFLSEAFTPPARQYGLAKLGFTQSYSYFTWRTTKWELTEFGNQIAELADYRRPNLFVNTPDILHAVLQHNGPGMFAIRAVLAATMSPAWGMYCGYELFEHRAVREGSEEYLDSEKYELRPRDFASALDQGRSLQPFITRLNIIRRLHPAFQQLRTIHFHHVDNDALLAYSKFDPATGDCVLVVVTLNAFGPEEATLWLDMAALGMEDYDRFWVRDEITGEEYQWGQANYIRIDPARAVAHIINMPAVPYESRNTLLRRRX

SEQ ID NO: 27 - protein, 260 aa, Mycobacterium tuberculosis
  misc_feature (260)..(260): Xaa can be any naturally occurring amino acid
MSKREDGRLDHELRPVIITRGFTENPAGSVLIEFGHTKVLCTASVTEGVPRWRKATGLGWLTAEYAMLPSATHSRSDRESVRGRLSGRTQEISRLIGRSLRACIDLAALGENTIAIDCDVLQADGGTRTAAITGAYVALADAVTYLSAAGKLSDPRPLSCAIAAVSVGVVDGRIRVDLPYEEDSRAEVDMNVVATDTGTLVEIQGTGEGATFARSTLDKLLDMALGACDTLFAAQRDALALPYPGVLPQGPPPPKAFGTX

SEQ ID NO: 28 - protein, 190 aa, Mycobacterium tuberculosis
LLGNTIHVPCQPCRHGHGAPSRGLRGRPADRWPVARATPTLHVCPQNQGVGLDFVRKPEYGRLRWPAYPAGTNNDRLISMRDGGIVALPQLTDEQRAAALEKAAAARRARAELKDRLKRGGTNLTQVLKDAESDEVLGKMKVSALLEALPKVGKVKAQEIMTELEIAPTRRLRGLGDRQRKALLEKFGSA

SEQ ID NO: 29 - protein, 419 aa, Mycobacterium tuberculosis
  misc_feature (419)..(419): Xaa can be any naturally occurring amino acid
MVDHKRIPKQVIVGVSGGIAAYKACTVVRQLTEASHRVRVIPTESALRFVGAATFEALSGEPVCTDVFADVPAVPHVHLGQQADLVVVAPATADLLARAAAGRADDLLTATLLTARCPVLFAPAMHTEMWLHPATVDNVATLRRRGAVVLEPATGRLTGADSGAGRLPEAEEITTLAQLLLERHDALPYDLAGRKLLVTAGGTREPIDPVRFIGNRSSGKQGYAVARVAAQRGADVTLIAGHTAGLVDPAGVEVVHVSSAQQLADAVSKHAPTADVLVMAAAVADFRPAQVATAKIKKGVEGPPTIELLRNDDVLAGVVRARAHGQLPNMRAIVGFAAETGDANGDVLFHARAKLRRKGCDLLVVNAVGEGRAFEVDSNDGWLLASDGTESALQHGSKTLMASRIVDAIVTFLAGCSSX

SEQ ID NO: 30 - protein, 2109 aa, Mycobacterium tuberculosis
  misc_feature (2109)..(2109): Xaa can be any naturally occurring amino acid
MGKERTKTVDRTRVTPVAVIGMGCRLPGGIDSPDRLWEALLRGDDLVTEIPADRWDIDEYYDPEPGVPGRTDCKWGAYLDNVGDFDPEFFGIGEKEAIAIDPQHRLLLETSWEAMEHGGLTPNQMASRTGVFVGLVHTDYILVHADNQTFEGPYGNTGTNACFASGRVAYAMGLQGPAITVDTACSSGLTAIHLACRSLHDGESDIALAGGVYVMLEPRRFASGSALGMLSATGRCHAFDVSADGFVSGEGCVMLALKRLPDALADGDRILAVIRGTAANQDGHTVNIATPSRSAQVAAYREALDVAGVDPATVGMVEAHGPGTPVGDPIEYASLAEVYGNDGPCALASVKTNFGHTQSAAGALGLMKAVLALQHGVVPQNLHFTALPDKLAAIETNLFVPQEITPWPGADQETPRRAAVSSYGMTGTNVHAIVEQAPVPAPESGAPGDTPATPGIDGALLFALSASSQDALRQTAARLADWVDAQGPELAPADLAYTLARRRGHRPVRTAVLAATTAELTEALREVATGEPPYPPAVGQDDRGPVWVFSGQGSQWAGMGADLLATEPVFAATIAAIEPLIAAESGFSVTEAMTAPEVVTGIDRVQPTLFAMQVALAATMKSYGVAPGAVIGHSLGESAAAVVAGALCLEDGVRVICRRSALMTRIAGAGAMASVELPAQQVLSELMARGVNDAVVAVVASPQSTVIGGATQTVRDLVAAWEQRDVLAREVAVDVASHSPQVDPILDELAEALAEISPLQPEIPYYSATSFDPREEPYCDAYYWVDNLRHTVRFAAAVQAALEDGYRVFTELTPHPLLTHAVDQTARSLDMSAAALAGMRREQPLPHGLRALAGDLYAAGAAVDFAVLYPTGRLINAPLPTWNHRRLLLDDTTRRIAHANTVAVHPLLGSHVRLPEEPERHVWQGEVGTVTQPWLADHQIHGAAALPGAAYCEMALAAARAVLGEASEVRDIRFEQMLLLDDETPIGVTATVEAPGVVPLTVETSHDGRYTRQLAAVLHVVREADDAPDQPPQKNIAELLASHPHKVDGAEVRQWLDKRGHRLGPAFAGLVDAYIAEGAGDTVLAEVNLPGPLRSQVKAYGVHPVLLDACFQSVAAHPAVQGMADGGLLLPLGVRRLRSYGSARHARYCCTTVTACGVGVEADLDVLDEHGAVVLAVRGLQLGTGASQASERARVLGERLLSIEWHERELPENSHAEPGAWLLISTCDATDLVAAQLTDALKVHDAQCTTMSWPQRADHAAQAARLRDQLGTGGFTGVFVLTAPQTGDPDAESPVRGGELVKHVVRIAREIPEITAQEPRLYVLTHNAQAVLSGDRPNLEQGGMRGLLRVIGAEHPHLKASYVDVDEQTGAESVARQLLAASGEDETAWRNDQWYTARLCPAPLRPEERQTTVVDHAEAGMRLQIRTPGDLQTLEFAAFDRVPPGPGEIEVAVTASSINFADVLVTFGRYQTLDGRQPQLGTDFAGVVSAVGPGVSELKVGDRVGGMSPNGCWATFVTCDARLATRLPEGLTDAQAAAVTTASATAWYGLQDLARIKAGDKVLIHSATGGVGQAAIAIARAAGAQIYATAGNEKRRDLLRDMGIEHVYDSRSVEFAEQIRRDTAGYGVDIVLNSVTGAAQLAGLKLLALGGRFIEIGKRDIYSNTRLELLPFRRNLAFYGLDLGLMSVSHPAAVRELLSTVYRLTVEGVLPMPQSTHYPLAEAATAIRVMGAAEHTGKLILDVPHAGRSSVVLPPEQARVFRSDGSYIITGGLGGLGLFLAEKMANAGAGRIVLSSRSQPSQKALETIELVRAIGSDVVVECGDIAQPDTADRLVTAATATGLPLRGVLHAAAVVEDATLANITDELIERDWAPKAYGAWQLHRATADQPLDWFCSFSSAAALVGSPGQGAYAAANSWLDTFTHWRRAQDLPATSIAWGAWGQIGRAIAFAEQTGDAIAPEEGAYAFETLLRHNRAYSGYAPVIGSPWLTAFAQHSPFAEKFQSLGQNRSGTSKFLAELVDLPREEWPDRLRRLLSKQVGLILRRTIDTDRLLSEYGLDSLSSQELRARVEAETGIRISATEINTTVRGLADLMCDKLAADRDAPAPAX

SEQ ID NO: 31 - protein, 766 aa, Mycobacterium tuberculosis
  misc_feature (766)..(766): Xaa can be any naturally occurring amino acid
MAFPVISTYRVQMRGRSNGFGFTFADAENLLDYLDDLGVSHLYLSPILTAVGGSTHGYDVTDPTTVSPELGGSDGLARLSAAARSRGMGLIVDIVPSHVGVGKPEQNAWWWDVLKFGRSSAYAEFFDIDWELGDGRIILPLLGSDSDVANLRVDGDLLRLGDLALPVAPGSGDGTGPAVHDRQHYRLVGWRHGLCGYRRFFSITSLAGLRQEDRAVFDASHAEVARWFTEGLVDGVRVDHLDGLSDPSGYLAQLRELLGPNAWIVVEKILAVDEALEPTLPVDGSTGYDVLREIGGVLVDPQGESPLTALVESAGVDYQEMPAMLADLKVHAAVHTLASELRRLRRCIAAAAGADHPLLPAAVAALLRHIGRYRCDYPGQAAVLPCALAETHSTTPQLAPGLQLIAAAVARGGEPAVRLQQLCGAVSAKAVEDCMFYRDARLVSLNEVGGEPRRFGVGAAEFHHRAATRARLWPRSMTTLSTHDTKRGEDVRARIGVLSQVPWLWAKFIGHAQAIAPAPDAVTGQFLWQNVFGVWPVSGEVSAALRGRLHTYAEKAIREAAWHTSWHNPNRAFEDDVHGWLDLVLDGPLASELTGLVAHLNSHAESDALAAKLLALTVPGVPDVYQGSELWDDSLVDPDNRRPVDYGTRRVALKALQHPKIRVLAAALRLRRTHPESFLGGAYHPVFAAGPAADHVVAFRRGDDILVAVTRWTVRLQQTGWDHTVLPLPDGSWTDALTGFTASGHTPAVELFADLPVVLLVRDNAX

SEQ ID NO: 32 - protein, 86 aa, Mycobacterium tuberculosis
  misc_feature (86)..(86): Xaa can be any naturally occurring amino acid
MPDEPTPPEATTPNSESDPRYDSAGVPTFESVREKIETRYGTALGATELDAESPQGRRLEDQYAQRQRAAAERLAQIRESMHTDEX

SEQ ID NO: 33 - protein, 312 aa, Mycobacterium tuberculosis
  misc_feature (312)..(312): Xaa can be any naturally occurring amino acid
MKTLALQLQGYLDHLTIERGVAANTLSSYRRDLRRYSKHLEERGITDLAKVGEHDVSEFLVALRRGDPDSGTAALSAVSAARALIAVRGLHRFAAAEGLAELDVARAVRPPTPSRRLPKSLTIDEVLSLLEGAGGDKPSDGPLTLRNRAVLELLYSTGARISEAVGLDLDDIDTHARSVLLRGKGGKQRLVPVGRPAVHALDAYLVRGRPDLARRGRGTAAIFLNARGGRLSRQSAWQVLQDAAERAGITAGVSPHMLRHSFATHLLEGGADVRVVQELLGHASVTTTQIYTLVTVHALREVWAGAHPRARX

SEQ ID NO: 34 - protein, 232 aa, Mycobacterium tuberculosis
  misc_feature (232)..(232): Xaa can be any naturally occurring amino acid
MTEHMPEHDPSYGIPDIAEPAELDADELKRVLEALLLVIDTPVTADALAAATEQPVYRVAAKLQLMADELTGRDSGIDLRHTSEGWRMYTRARFAPYVEKLLLDGARTKLTRAALETLAVVAYRQPVTRARVSAVRGVNVDAVMRTLLARGLITEVGTDADTGAVTFATTELFLERLGLTSLSELPDIAPLLPDVDTIDDLSESLDSEPRFIKLTGELASEQTLSFDVDRDX

SEQ ID NO: 35 - protein, 415 aa, Mycobacterium tuberculosis
  misc_feature (415)..(415): Xaa can be any naturally occurring amino acid
MHEVAAREQRSDGPMRLDAQGRLQRYEEAFADYDAPFAFVDLDAMWGNADQLLARAGDKPIRVASKSLRCRPLQREILDASERFDGLLTFTLTETLWLAGQGFSNLLLAYPPTDRAALRALGELTAKDPDGAPIVMVDSVEHLDLIERTTDKPVRLCLDFDAGYWRAGGRIKIGSKRSPLHTPEQARALAVEIARRPALTLAALMCYEAHIAGLGDNVAGKRVHNAIIRRMQRMSFEELRERRARAVELVREVADIKIVNAGGTGDLQLVAQEPLITEATAGSGFYAPTLFDSYSTFTLQPAAMFALPVCRRPGAKTVTALGGGYLASGVGAKDRMPTPYLPVGLKLNALEGTGEVQTPLSGDAARRLKLGDKVYFRHTKAGELCERFDHLHLVRGAEVVDTVPTYRGEGRTFLX

SEQ ID NO: 36 - protein, 188 aa, Mycobacterium tuberculosis
  misc_feature (188)..(188): Xaa can be any naturally occurring amino acid
MQNHDYVTYEEFGRRFFEVAVTPDRVAAAFADIAGSEFAMEPISQGPGGIAKVSANVKIREPRVTRKLGDLITFVIHIPLSIDLLLDLRLDKQRFMVAGDIALRATARAAEPLLLIVDVAKPRPSDITVNVSSKSIRGEVLRILAGVDGEIRRFIAQYVSAEIDSPKSQAAQVINVAEQLDSTWSGPX

SEQ ID NO: 37 - protein, 401 aa, Mycobacterium tuberculosis
  misc_feature (401)..(401): Xaa can be any naturally occurring amino acid
MTRVVVIGSGFAGLWAALGAARRLDELAVLAGTVDVMVVSNKPFHDIRVRNYEADLSACRIPLGDVLGPAGVAHVTAEVTAIDADGRRVTTSTGASYSYDRLVLASGSHVVKPALPGLAEFGFDVDTYDGAVRLQQHLQGLAGGPLTSAAATVVVVGAGLTGIETACELPGRLHALFARGDGVTPRVVLIDHNPFVGSDMGLSARPVIEQALLDNGVETRTGVSVAAVSPGGVTLSSGERLAAATVVWCAGMRASRLTEQLPVARDRLGRLQVDDYLRVIGVPAMFAAGDVAAARMDDEHLSVMSCQHGRPMGRYAGCNVINDLFDQPLLALRIPWYVTVLDLGSAGAVYTEGWERKVVSQGAPAKTTKQSINTRRIYPPLNGSRADLLAAAAPRVQPRPX

SEQ ID NO: 38 - protein, 640 aa, Mycobacterium tuberculosis
  misc_feature (640)..(640): Xaa can be any naturally occurring amino acid
MGPKLFKPSIDWSRAFPDSVYWVGKAWTISAICVLAILVLLRYLTPWGRQFWRITRAYFVGPNSVRVWLMLGVLLLSVVLAVRLNVLFSYQGNDMYTALQKAFEGIASGDGTVKRSGVRGFWMSIGVFSVMAVLHVTRVMADIYLTQRFIIAWRVWLTHHLTQDWLDGRAYYRDLFIDETIDNPDQRIQQDVDIFTAGAGGTPNAPSNGTASTLLFGAVQSIISVISFTAILWNLSGTLNIFGVSIPRAMFWTVLVYVFVATVISFIIGRPLIWLSFRNEKLNAAFRYALVRLRDAAEAVGFYRGERVEGTQLQRRFTPVIDNYRRYVRRSIAFNGWNLSVSQTIVPLPWVIQAPRLFAGQIDFGDVGQTATSFGNIHDSLSFFRNNYDAFASFRAAIIRLHGLVDANEKGRALPAVLTRPSDDESVELNDIEVRTPAGDRLIDPLDVRLDRGGSLVITGRSGAGKTTLLRSLAELWPYASGTLHRPGGENETMFLSQLPYVPLGTLRDVVCYPNSAAAIPDATLRDTLTKVALAPLCDRLDEERDWAKVLSPGEQQRVAFARILLTKPKAVFLDESTSALDTGLEFALYQLLRSELPDCIVISVSHRPALERLHENQLELLGGGQWRLAPVEAAPAEVX

SEQ ID NO: 39 - protein, 287 aa, Mycobacterium tuberculosis
  misc_feature (287)..(287): Xaa can be any naturally occurring amino acid
MPGRTSIGVKIRDKVQDKVIAITGGARGIGLATAAALHNLGAKVAIGDIDEAMAKESGADLDLDMYGKLDVTDPDSFSGFLDAVERQLGPIDVLVNNAGIMPVGRIVDEPDPVTRRILDINVYGVILGSKLAAQRMVPRGRGHVINVASLAGEIYAVGVATYCASKHAVVAFTDSARLEYRSAGVKFSMVLPSFVNTELIAGTGGIKGFKNAEPADIADAIVGLIVHPKPRVRVTKAAGSMIVAQRFMPRQVSEGLNRLLGGEHVFTDDVDMEKRRTYEARARGEEX

SEQ ID NO: 40 - protein, 278 aa, Mycobacterium tuberculosis
  misc_feature (278)..(278): Xaa can be any naturally occurring amino acid
MKAIFITGAGSGMGREGATLFHANGWRVGAIDRNEDGLAALRVQLGAERLWARAVDVTDKAALEGALADFCAGNVGGGLDMMWNNAGIGEGGWFEDVPYEAAVRVVDVNFKAVLTGAYAALPYLKKAPGSLMFSTSSSSGTYGMPRIAVYSATKHAVKGLTEALSVEWQRHGVRVADVLPGLIDTAILTSTRQHSDEGPYTISAEQIRAAAPKKGMFRLMPSSSVAEAAWRAYQHPTRLHWYVPRSIRWIDRLKGVSPEFVRRHIAKSLATLEPKRKX

SEQ ID NO: 41 - protein, 516 aa, Mycobacterium tuberculosis
  misc_feature (516)..(516): Xaa can be any naturally occurring amino acid
MGSVHDVIEAFRKAPSNAERGTKFEQLMVRYFELDPTMAQQYDAVWWWIDWPERRGRTDTGIDLVARERDTGNYTAIQCKFYEPTHTLAKGDIDSFFTASGKTGFTNRVIISTTDRWGRNAEDALADQLVPVQRIGMAEIAESPIDWDIAWPADDLQVNLTPAKRHELRPHQQQAIDAVFRGFAVGNDRGKLIMACGTGKTFTALKIAERIAADNGGSARILLLVPSISLLSQTLREWTAQSELDVRAFAVCSDTKVSRSAEDYHVHDVPIPVTTDARVLLHEMAHRRRAQGLTVVFCTYQSLPTVAKAQRLGVDEFDLVMCDEAHRTTGVTLAGDDESNFVRVHDGQYLKAARRLYMTATPRIFTESIKDRADQHSAELVSMDDELTFGPEFHRLSFGEAVERGLLTDYKVMVLTVDQGVIAPRLQQELSGVSGELMLDDASKIVGCWNGLAKRSGTGIVAGEPPMRRAVAFAKDIKTSKQVAELFPKVVEAYRELVDDGPGLACLNSSRRIQAX

SEQ ID NO: 42 - protein, 219 aa, Mycobacterium tuberculosis
  misc_feature (219)..(219): Xaa can be any naturally occurring amino acid
MRIAALVAVSLLIAGCSREVGGDVGQSQTIAPPAPAPSAAPSTPPAAGAPITTIVSWIEAGHPVDPAAYHVATRDGVTTQLGDDVAFSASSGTVACMTDARHTSGTLACLVRLANPPPRPETAYGEWKGGWVDFDGIHLQVGSARADPGPFVYGNGPELANGDTLSIGDYRCRSYQAGLFCVNYAHQSAVRFASAGIEPFGCLKPAPPPDGVGVAFGCX

SEQ ID NO: 43 - protein, 1195 aa, Mycobacterium tuberculosis
  misc_feature (1195)..(1195): Xaa can be any naturally occurring amino acid
MPEPTVLLLSTSDTDLISARSSGKNYRWANPSRLSDLELTDLLAEASIVVIRILGGYRAWQSGIDTVIAGGVPAVLVSGEQAADAELTDRSTVAAGTALQAHIYLAHGGVDNLRELHAFLCDTVLMTGFGFTPPVATPTWGVLERPDAGKTGPTIAVLYYRAQHLAGNTGYVEALCRAIEDAGGRPLPLYCASLRTAEPRLLERLGGADAMVVTVLAAGGVKPAAASAGGDDDSWNVEHLAALDIPILQGLCLTSPRDQWCANDDGLSPLDVASQVAVPEFDGRIITVPFSFKEIDDDGLISYVADPERCARVAGLAVRHARLRQVAPADKRVALVFSAYPTKHARIGNAVGLDTPASAVALLQAMRQRGYRVGDLPGVESNDGDALIHALIECGGHDPDWLTEGQLAGNPIRVSAKEYRDWFATLPAELTDVVTAYWGPPPGELFVDRSHDPDGEIVIAALRAGNLVLMVQPPRGFGENPVAIYHDPDLPPSHHYLAAYRWLDTGFSNGFGAHAVVHLGKHGNLEWLPGKTLGMSASCGPDAALGDLPLIYPFLVNDPGEGTQAKRRAHAVLVDHLIPPMARAETYGDIARLEQLLDEHASVAALDPGKLPAIRQQIWTLIRAAKMDHDLGLTERPEEDSFDDMLLHVDGWLCEIKDVQIRDGLHILGQNPTGEQELDLVLAILRARQLFGGAHAIPGLRQALGLAEDGTDERATVDQTEAKARELVAALQATGWDPSAADRLTGNADAAAVLRFAATEVIPRLAGTATEIEQVLRALDGRFIPAGPSGSPLRGLVNVLPTGRNFYSVDPKAVPSRLAWEAGVALADSLLARYRDEHGRWPRSVGLSVWGTSAMRTAGDDIAEVLALLGVRPVWDDASRRVIDLAPMQPAELGRPRIDVTVRISGFFRDAFPHVVTMLDDAVRLVADLDEAAEDNYVRAHAQADLAHHGDQRRATTRIFGSKPGTYGAGLLQLIDSRSWRDDADLAQVYTAWGGFAYGRDLDGREAIDDMNRQYRRIAVAAKNTDTREHDIADSDDYFQYHGGMVATVRALTGQAPAAYIGDNTRPDAIRTRTLSEETTRVFRARVVNPRWMAAMRRHGYKGAFEMAATVDYLFGYDATAGVMADWMYEQLTQRYVLDAQNRTFMTESNPWALHGMAERLLEAAGRGLWAQPAPETLDGLRQVLLETEGDLEAX

SEQ ID NO: 44 - protein, 138 aa, Mycobacterium tuberculosis
  misc_feature (138)..(138): Xaa can be any naturally occurring amino acid
MAMVNTTTRLSDDALAFLSERHLAMLTTLRADNSPHVVAVGFTFDPKTHIARVITTGGSQKAVNADRSGLAVLSQVDGARWLSLEGRAAVNSDIDAVRDAELRYAQRYRTPRPNPRRVVIEVQIERVLGSADLLDRAX

SEQ ID NO: 45 - protein, 394 aa, Mycobacterium tuberculosis
  misc_feature (394)..(394): Xaa can be any naturally occurring amino acid
MPAPDPMRGDPPHPAPPRLRSPLDPTSGDPLHPAPPRLRSPLDPTSGDPLHPAPPRLRSPLDPTSGDPLHPAPPRLRSPLVLLDGASMWFRSFFGVPSSITAPDGRPVNAVRGFIDSMAVVITQQRPNRLAVCLDLDWRPQFRVDLIPSYKAHRVAEPEPNGQPDVEEVPDELTPQVDMIMELLDAFGIAMAGAPGFEADDVLGTLATRERRDPVIVVSGDRDLLQVVADDPVPVRVLYLGRGLAKATLFGPAEVAERYGLPAHRAGAAYAELALLRGDPSDGLPGVPGVGEKTAATLLARHGSLDQIMAAADDRKTTMAKGLRTKLLAASAYIKAADRVVRVATDAPVTLSTPTDRFPLVAADPERTAELATRFGVESSIARLQKALDTLPGX

SEQ ID NO: 46 - protein, 292 aa, Mycobacterium tuberculosis
  misc_feature (292)..(292): Xaa can be any naturally occurring amino acid
MTWPLPDRLSINSLSGTPAVDLSSFTDFLRRQAPELLPASISGGAPLAGGDAQLPHGTTIVALKYPGGVVMAGDRRSTQGNMISGRDVRKVYITDDYTATGIAGTAAVAVEFARLYAVELEHYEKLEGVPLTFAGKINRLAIMVRGNLAAAMQGLLALPLLAGYDIHASDPQSAGRIVSFDAAGGWNIEEEGYQAVGSGSLFAKSSMKKLYSQVTDGDSGLRVAVEALYDAADDDSATGGPDLVRGIFPTAVIIDADGAVDVPESRIAELARAIIESRSGADTFGSDGGEKX

SEQ ID NO: 47 - protein, 268 aa, Mycobacterium tuberculosis
  misc_feature (268)..(268): Xaa can be any naturally occurring amino acid
MVSPAAPDLTDDLTDAELAADLAADAGKLLLQVRAEIGFDQPWTLGEAGDRQANSLLLRRLQAERPGDAVLSEEAHDDLARLKSDRVWIIDPLDGTREFSTPGRDDWAVHIALWRRSSNGQPEITDAAVALPARGNVVYRTDTVTSGAAPAGVPGTLRIAVSATRPPAVLHRIRQTLAIQPVSIGSAGAKAMAVIDGYVDAYLHAGGQWEWDSAAPAGVMLAAGMHASRLDGSPLRYNQLDPYLPDLLMCRAEVAPILLGAIADAWRX

SEQ ID NO: 48 - protein, 411 aa, Mycobacterium tuberculosis
  misc_feature (411)..(411): Xaa can be any naturally occurring amino acid
MKDTVSQPAGGRGATAPRPADAASPSCGSSPSADSVSVVLAGGGTAGHVEPAMAVADALVALDPRVRITALGTLRGLETRLVPQRGYHLELITAVPMPRKPGGDLARLPSRVWRAVREARDVLDDVDADVVVGFGGYVALPAYLAARGLPLPPRRRRRIPVVIHEANARAGLANRVGAHTADRVLSAVPDSGLRRAEVVGVPVRASIAALDRAVLRAEARAHFGFPDDARVLLVFGGSQGAVSLNRAVSGAAADLAAAGVCVLHAHGPQNVLELRRRAQGDPPYVAVPYLDRMELAYAAADLVICRAGAMTVAEVSAVGLPAIYVPLPIGNGEQRLNALPVVNAGGGMVVADAALTPELVARQVAGLLTDPARLAAMTAAAARVGHRDAAGQVARAALAVATGAGARTTTX

SEQ ID NO: 49 - protein, 144 aa, Mycobacterium tuberculosis
  misc_feature (144)..(144): Xaa can be any naturally occurring amino acid
MFLGTYTPKLDDKGRLTLPAKFRDALAGGLMVTKSQDHSLAVYPRAAFEQLARRASKAPRSNPEARAFLRNLAAGTDEQHPDSQGRITLSADHRRYASLSKDCVVIGAVDYLEIWDAQAWQNYQQIHEENFSAASDEALGDIFX

SEQ ID NO: 50 - protein, 380 aa, Mycobacterium tuberculosis
  misc_feature (380)..(380): Xaa can be any naturally occurring amino acid
MVVSTDQAHSLGDVLGIAVPPTGQGDPVRVLAYDPEAGGGFLDALALDTLALLEGRWLHVVETLDRRFPGSELSSIAPEELCALPGIQEVLGLHAVGELAAARRWDRIVVDCASTADALRMLTLPATFGLYVERAWPRHRRLSIGADDGRSAVLAELLERIRASVERLSTLLTDGALVSAHLVLTPERVVAAEAVRTLGSLALMGVRVEELLVNQLLVQDENYEYRSLPDHPAFHWYAERIGEQRAVLDDLDATIGDVALVLVPHLAGEPIGPKALGGLLDSARRRQGSAPPGPLQPIVDLESGSGLASIYRLRLALPQLDPGTLTLGRADDDLIVSAGGMRRRVRLASVLRRCTVLDAHLRGGELTVRFRPNPEVWPTX

SEQ ID NO: 51 - protein, 371 aa, Mycobacterium tuberculosis
  misc_feature (371)..(371): Xaa can be any naturally occurring amino acid
MALSAEGSSGGSRGGSPKAEAASVPSWPQILGRLTDNRDLARGQAAWAMDQIMTGNARPAQIAAFAVAMTMKAPTADEVGELAGVMLSHAHPLPADTVPDDAVDVVGTGGDGVNTVNLSTMAAIVVAAAGVPVVKHGNRAASSLSGGADTLEALGVRIDLGPDLVARSLAEVGIGFCFAPRFHPSYRHAAAVRREIGVPTVFNLLGPLTNPARPRAGLIGCAFADLAEVMAGVFAARRSSVLVVHGDDGLDELTTTTTSTIWRVAAGSVDKLTFDPAGFGFARAQLDQLAGGDAQANAAAVRAVLGGARGPVRDAVVLNAAGAIVAHAGLSSRAEWLPAWEEGLRRASAAIDTGAAEQLLARWVRFGRQIX

SEQ ID NO: 52 - protein, 231 aa, Mycobacterium tuberculosis
  misc_feature (231)..(231): Xaa can be any naturally occurring amino acid
MPGPHSPNPGVGTNGPAPYPEPSSHEPQALDYPHDLGAAEPAFAPGPADDAALPPAAYPGVPPQVSYPKRRHKRLLIGIVVALALVSAMTAAIIYGVRTNGANTAGTFSEGPAKTAIQGYLNALENRDVDTIVRNALCGIHDGVRDKRSDQALAKLSSDAFRKQFSQVEVTSIDKIVYWSQYQAQVLFTMQVTPAAGGPPRGQVQGIAQLLFQRGQVLVCSYVLRTAGSYX

SEQ ID NO: 53 - protein, 292 aa, Mycobacterium tuberculosis
  misc_feature (292)..(292): Xaa can be any naturally occurring amino acid
MSSPRERRPASQAPRLSRRPPAHQTSRSSPDTTAPTGSGLSNRFVNDNGIVTDTTASGTNCPPPPRAAARRASSPGESPQLVIFDLDGTLTDSARGIVSSFRHALNHIGAPVPEGDLATHIVGPPMHETLRAMGLGESAEEAIVAYRADYSARGWAMNSLFDGIGPLLADLRTAGVRLAVATSKAEPTARRILRHFGIEQHFEVIAGASTDGSRGSKVDVLAHALAQLRPLPERLVMVGDRSHDVDGAAAHGIDTVVVGWGYGRADFIDKTSTTVVTHAATIDELREALGVX

SEQ ID NO: 54 - protein, 261 aa, Mycobacterium tuberculosis
  misc_feature (261)..(261): Xaa can be any naturally occurring amino acid
MPKSRRAVSLSVLIGAVIAALAGALIAVTVPARPNRPEADREALWKIVHDRCEFGYRRTGAYAPCTFVDEQSGTALYKADFDPYQFLLIPLARITGIEDPALRESAGRNYLYDAWAARFLVTARLNNSLPESDVVLTINPKNARTQDQLHIHISCSSPTTSAALRNVDTSEYVGWKQLPIDLGGRRFQGLAVDTKAFESRNLFRDIYLKVTADGKKMENASIAVANVAQDQFLLLLAEGTEDQPVAAETLQDHDCSITKSX

SEQ ID NO: 55 - protein, 698 aa, Mycobacterium tuberculosis
  misc_feature (698)..(698): Xaa can be any naturally occurring amino acid
MCCAVCGPEPGRIGEVTPLGPCPAQHRGGPLRPSELAQASVMAALCAVTAIISVVVPFAAGLALLGTVPTGLLAYRYRLRVLAAATVAAGMIAFLIAGLGGFMGVVHSAYIGGLTGIVKRRGRGTPTVVVSSLIGGFVFGAAMVGMLAAMVRLRHLIFKVMTANVDGIAATLARMHMQGAAADVKRYFAEGLQYWPWVLLGYFNIGIMIVSLIGWWALSRLLERMRGIPDVHKLDPPPGDDVDALIGPVPVRLDKVRFRYPRAGQDALREVSLDVRAGEHLAIIGANGSGKTTLMLILAGRAPTSGTVDRPGTVGLGKLGGTAVVLQHPESQVLGTRVADDVVWGLPLGTTADVGRLLSEVGLEALAERDTGSLSGGELQRLALAAALAREPAMLIADEVTTMVDQQGRDALLAVLSGLTQRHRTALVHITHYDNEADSADRTLSLSDSPDNTDMVHTAAMPAPVIGVDQPQHAPALELVGVGHEYASGTPWAKTALRDINFVVEQGDGVLIHGGNGSGKSTLAWIMAGLTIPTTGACLLDGRPTHEQVGAVALSFQAARLQLMRSRVDLEVASAAGFSASEQDRVAAALTVVGLDPALGARRIDQLSGGQMRRVVLAGLLARAPRALILDEPLAGLDAASQRGLLRLLEDLRRARGLTVVVVSHDFAGMEELCPRTLHLRDGVLESAAASEAGGMSX

SEQ ID NO: 56 - protein, 311 aa, Mycobacterium tuberculosis
  misc_feature (311)..(311): Xaa can be any naturally occurring amino acid
MSIAEDITQLIGRTPLVRLRRVTDGAVADIVAKLEFFNPANSVKDRIGVAMLQAAEQAGLIKPDTIILEPTSGNTGIALAMVCAARGYRCVLTMPETMSLERRMLLRAYGAELILTPGADGMSGAIAKAEELAKTDQRYFVPQQFENPANPAIHRVTTAEEVWRDTDGKVDIVVAGVGTGGTITGVAQVIKERKPSARFVAVEPAASPVLSGGQKGPHPIQGIGAGFVPPVLDQDLVDEIITVGNEDALNVARRLAREEGLLVGISSGAATVAALQVARRPENAGKLIVVVLPDFGERYLSTPLFADVADX

SEQ ID NO: 57 - protein, 317 aa, Mycobacterium tuberculosis
  misc_feature (317)..(317): Xaa can be any naturally occurring amino acid
MHLVLGDEELLVERAVADVLRSARQRAGTADVPVSRMRAGDVGAYELAELLSPSLFAEERIVVLGAAAEAGKDAAAVIESAAADLPAGTVLVVVHSGGGRAKSLANQLRSMGAQVHPCARITKVSERADFIRSEFASLRVKVDDETVTALLDAVGSDVRELASACSQLVADTGGAVDAAAVRRYHSGKAEVRGFDIADKAVAGDVAGAAEALRWAMMRGEPLVVLADALAEAVHTIGRVGPQSGDPYRLAAQLGMPPWRVQKAQKQARRWSRDTVATAMRLVAELNANVKGAVADADYALESAVRQVAELVADRGRX

SEQ ID NO: 58 - protein, 171 aa, Mycobacterium tuberculosis
  misc_feature (171)..(171): Xaa can be any naturally occurring amino acid
MVPAQHRPPDRPGDPAHDPGRGRRLGIDVGAARIGVACSDPDAILATPVETVRRDRSGKHLRRLAALAAELEAVEVIVGLPRTLADRIGRSAQDAIELAEALARRVSPTPVRLADERLTTVSAQRSLRQAGVRASEQRAVIDQAAAVAILQSWLDERLAAMAGTQEGSDAX

SEQ ID NO: 59 - protein, 98 aa, Mycobacterium tuberculosis
  misc_feature (98)..(98): Xaa can be any naturally occurring amino acid
MGIQRAVLLIADIGGYTNYMHWNRKHLAHAQWTVAQLLESVIDAAKGMKLAKLEGDAAFFWAPGGQHQCPGMRPAPADAPEVPHAARADQKRPSLRLX

SEQ ID NO: 60 - protein, 443 aa, Mycobacterium tuberculosis
  misc_feature (443)..(443): Xaa can be any naturally occurring amino acid
MASKAKTGRDDEATSAVELTEATESAVARTDGDSTTDTASKLGHHSFLSRLYTGTGAFEVVGRRRLWFGVSGAIVAVAIASIVFRGFTFGIDFKGGTTVSFPRGSTQVAQVEDVYYRALGSEPQSVVIVGAGASATVQIRSETLTSDQTAKLRDALFEAFGPKGTDGQPSKQAISDSAVSETWGGQITKKAVIALVVFLVLVALYITVRYERYMTISAITAMLFDLTVTAGVYSLVGFEVTPATVIGLLTILGFSLYDTVIVFDKVEENTHGFQHTTRRTFAEQANLAINQTFMRSINTSLIGVLPVLALMVVAVWLLGVGTLKDLALVQLIGIIIGTYSSIFFATPLLVTLRERTELVRNHTRRVLKRRNSGSPAGSEDASTDGGEQPAAADEQSLVGITQASSQSAPRAAQGSSKPAPGARPVRPVGTRRPTGKRNAGRRX

SEQ ID NO: 61 - protein, 300 aa, Mycobacterium tuberculosis
  misc_feature (300)..(300): Xaa can be any naturally occurring amino acid
MDPAGNPATGTARVKRGMAEMLKGGVIMDVVTPEQARIAEGAGAVAVMALERVPADIRAQGGVSRMSDPDMIEGIIAAVTIPVMAKVRIGHFVEAQILQTLGVDYIDESEVLTPADYAHHIDKWNFTVPFVCGATNLGEALRRISEGAAMIRSKGEAGTGDVSNATTHMRAIGGEIRRLTSMSEDELFVAAKELQAPYELVAEVARAGKLPVTLFTAGGIATPADAAMMMQLGAEGVFVGSGIFKSGAPEHRAAAIVKATTFFDDPDVLAKVSRGLGEAMVGINVDEIAVGHRLAQRGWX

SEQ ID NO: 62 - protein, 791 aa, Mycobacterium tuberculosis
  misc_feature (791)..(791): Xaa can be any naturally occurring amino acid
MTQTPDREKALELAVAQIEKSYGKGSVMRLGDEARQPISVIPTGSIALDVALGIGGLPRGRVIEIYGPESSGKTTVALHAVANAQAAGGVAAFIDAEHALDPDYAKKLGVDTDSLLVSQPDTGEQALEIADMLIRSGALDIVVIDSVAALVPRAELEGEMGDSHVGLQARLMSQALRKMTGALNNSGTTAIFINQLRDKIGVMFGSPETTTGGKALKFYASVRMDVRRVETLKDGTNAVGNRTRVKVVKNKCLAEGTRIFDPVTGTTHRIEDVVDGRKPIHVVAAAKDGTLHARPVVSWFDQGTRDVIGLRIAGGAIVWATPDHKVLTEYGWRAAGELRKGDRVAQPRRFDGFGDSAPIPADHARLLGYLIGDGRDGWVGGKTPINFINVQRALIDDVTRIAATLGCAAHPQGRISLAIAHRPGERNGVADLCQQAGIYGKLAWEKTIPNWFFEPDIAADIVGNLLFGLFESDGWVSREQTGALRVGYTTTSEQLAHQIHWLLLRFGVGSTVRDYDPTQKRPSIVNGRRIQSKRQVFEVRISGMDNVTAFAESVPMWGPRGAALIQAIPEATQGRRRGSQATYLAAEMTDAVLNYLDERGVTAQEAAAMIGVASGDPRGGMKQVLGASRLRRDRVQALADALDDKFLHDMLAEELRYSVIREVLPTRRARTFDLEVEELHTLVAEGVVVHNCSPPFKQAEFDILYGKGISREGSLIDMGVDQGLIRKSGAWFTYEGEQLGQGKENARNFLVENADVADEIEKKIKEKLGIGAVVTDDPSNDGVLPAPVDFX

SEQ ID NO: 63 - protein, 389 aa, Mycobacterium tuberculosis
  misc_feature (389)..(389): Xaa can be any naturally occurring amino acid
MRVAVVAGPDPGHSFPAIALCQRFRAAADTPTLFTGVEWLEAARAAGIDAVELDGLAATDRDLDAGARIHRRAAQMAVLNVPRLRALEPELVVSDVITACGGMAAELLGIPWVELNPHPLYLPSKGLPPIGSGLAAGTGIRGRLRDATMRALTGRSWRAGLRQRAAVRVEIGLPARDPGPLRRLIATLPALEVPRPDWPAEAVVVGPLHFEPTDRVLAIPAGTGPVVVVAPSTALTGTAGLTEVALQSLTPGETVPSGSRLVVSRLSGADLTVPPWAVAGLGSQAELLTRADLVICGGGHGMVAKTLLAGVPMVVVPGGGDQWEIANRVVRQGSAVLIRPLTADALVAAVNEVLSSPRFREAARRAAASVAGAADPVRVCHDALALAGX

SEQ ID NO: 64 - protein, 157 aa, Mycobacterium tuberculosis
  misc_feature (157)..(157): Xaa can be any naturally occurring amino acid
MPDPDGPSVTVTVEIDANPDLVYGLITDLPTLASLAEEVVAMQLRKGDDVRKGAVFVGRNENGGRRWTTTCTVTDADPGRVFAFDVRSGIIPISRWQYGIVATEHGCRVTESTWDRRPSWFRAVARMATGVKDRASVNTEHIRRTLQRLKDRAEAGX

SEQ ID NO: 65 - protein, 752 aa, Mycobacterium tuberculosis
MSAAEIDEGVFETTATIDNGSFGTRTIRFETGRLALQAAGAVVAYLDDDNMLLSATTASKNPKEHFDFFPLTVDVEERMYAAGRIPGSFFRREGRPSTDAILTCRLIDRPLRPSFVDGLRNEIQIVVTILSLDPGDLYDVLAINAASASTQLGGLPFSGPIGGVRVALIDGTWVGFPTVDQIERAVFDMVVAGRIVEGDVAIMMVEAEATENVVELVEGGAQAPTESVVAAGLEAAKPFIAALCTAQQELADAAGKSGKPTVDFPVFPDYGEDVYYSVSSVATDELAAALTIGGKAERDQRIDEIKTQVVQRLADTYEGREKEVGAALRALTKKLVRQRILTDHFRIDGRGITDIRALSAEVAVVPRAHGSALFERGETQILGVTTLDMIKMAQQIDSLGPETSKRYMHHYNFPPFSTGETGRVGSPKRREIGHGALAERALVPVLPSVEEFPYAIRQVSEALGSNGSTSMGSVCASTLALLNAGVPLKAPVAGIAMGLVSDDIQVEGAVDGVVERRFVTLTDILGAEDAFGDMDFKVAGTKDFVTALQLDTKLDGIPSQVLAGALEQAKDARLTILEVMAEAIDRPDEMSPYAPRVTTIKVPVDKIGEVIGPKGKVINAITEETGAQISIEDDGTVFVGATDGPSAQAAIDKINAIANPQLPTVGERFLGTVVKTTDFGAFVSLLPGRDGLVHISKLGKGKRIAKVEDVVNVGDKLRVEIADIDKRGKISLILVADEDSTAAATDAATVTS

SEQ ID NO: 66 - protein, 588 aa, Mycobacterium tuberculosis
  misc_feature (588)..(588): Xaa can be any naturally occurring amino acid
MSTFRECRSMFDAAVKSYQSGDLANARAAFGRLTVENPDMSDGWLGLLACGDHHLDTLAGAHQHSEALYSETRRVGLTDGELSAVVMAPMYLGLRVWSRATIGLAYASALIIADRHDEAAATLDDPVITEDTGAAQYRQFVMATLFHKTRSWSNLLKVTEISPPSGATDVRDEVADAVAALASTAAASLGQFQFALELAEQVSTTNPRVTADVTLTRAWCLRELGDDDAARVALSATTTGDAPRTNTTAEQAGSPQPKFRHPYDDGRDLLVARRRPPAGDGWRKAVTKMTFGRVNPEPSAKREQTDELIQRICAPLADVHKLAFVSAKGGVGKTTMTVLVGNAVARLRGDRVMAVDVDADLGDLSARFSERGGPQTNIEHFVSSQHTKRYADVRVHTVMNKDRLEMLGAQNDPRSTYKFGPEDYGAAMQILETHCNVILLDCGTPVNGPLFSNILNDVTGLVVVASEDVRGVEGALVTLDWLGAHGFGRLLQHTVVVLNAIQKTRSLVDCGAAENQFRKRVPDFFRIPYDPHLATGLAVDFSSLKRRTRNAVLDLAGGLAQHYPASRVRPRGEDSWKTWIETMRQVGX

SEQ ID NO: 67 - protein, 456 aa, Mycobacterium tuberculosis
  misc_feature (456)..(456): Xaa can be any naturally occurring amino acid
MSTTQLINPATEEVLASVDHTDANAVDDAVQRARAAQRRWARLAPAQRAAGLRAFAAAVQAHLDELAALEVANSGHPIVSAEWEAGHVRDVLAFYAASPERLSGRQIPVAGGVDVTFNEPMGVVGVITPWNFPMVIASWAIAPALAAGNAVLVKPAELTPLTTMRLGELAVEAGLDEDLLQVLPGKGTVVGERFVTHPDIRKIVFTGSTEVGKRVMAGAAAQVKRVTLELGGKSANIVFHDCDLERAATTAPAGVFDNAGQDCCARSRILVQRSVYDRFMELLEPAVHSIVVGDPGSRATEMGPLVSRAHRDKVAGYVPDDAPVAFRGTAPAGRGFWFPPTVLTPKRGDRTVTDEIFGPVVVVLTFDDEADAISLANDTAYGLSGSIWTDDLSRALRVARAVESGNLSVNSHSSVRFNTPFGGFKQSGVGRELGPDAPLQFTETKNVFIAVGEEMX

SEQ ID NO: 68 - protein, 612 aa, Mycobacterium tuberculosis
  misc_feature (612)..(612): Xaa can be any naturally occurring amino acid
MLAWRQLNDLEETVTYDVIIRDGLWFDGTGNAPLTRTLGIRDGVVATVAAGALDETGCPEVVDAAGKWVVPGFIDVHTHYDAEVLLDPGLRESVRHGVTTVLLGNCSLSTVYANSEDAADLFSRVEAVPREFVLGALRDNQTWSTPAEYIEAIDALPLGPNVSSLLGHSDLRTAVLGLDRATDDTVRPTEAELAKMAKLLDEALEAGMLGMSGMDAAIDKLDGDRFRSRALPSTFATWRERRKLISVLRHRGRILQSAPDVDNPVSALLFFLASSRIFNRRKGVRMSMLVSADAKSMPLAVHVFGLGTRVLNKLLGSQVRFQHLPVPFELYSDGIDLPVFEEFGAGTAALHLRDQLQRNELLADRSYRRSFRREFDRIKLGPSLWHRDFHDAVIVECPDKSLIGKSFGAIADERGLHPLDAFLDVLVDNGERNVRWTTIVANHRPNQLNKLAAEPSVHMGFSDAGAHLRNMAFYNFGLRLLKRARDADRAGQPFLSIERAVYRLTGELAEWFGIGAGTLRQGDRADFAVIDPTHLDESVDGYHEEAVPYYGGLRRMVNRNDATVVATGVGGTVVFRGGQFGGQFRDGYGQNVKSGRYLRAGELGAALSRSAX

SEQ ID NO: 69 - protein, 423 aa, Mycobacterium tuberculosis
  misc_feature (423)..(423): Xaa can be any naturally occurring amino acid
MWEGLWIATAVIAALVVIAALTLGLVLYRRRRISLSPRPERGVVDRSGGYTASSGITFSQTPTTQPAERIDTSGLPAVGDDATVPRDAPKRTIADVHLPEFEPEPQAPEVPEADAIAPPEGRLERLRGRLARSQNALGRGLLGLIGGGDLDEDSWQDVEDTLLVADLGPAATASVVSQLRSRLASGNVRTEADARAVLRDVLINELQPGMDRSIRALPHAGHPSVLLVVGVNGTGKTTTVGKLARVLVADGRRVVLGAADTFRAAAADQLQTWAARVGAAVVRGPEGADPASVAFDAVDKGIAAGADVVLIDTAGRLHTKVGLMDELDKVKRVVTRRASVDEVLLVLDATIGQNGLAQARVFAEVVDISGAVLTKLDGTAKGGIVFRVQQELGVPVKLVGLGEGPDDLAPFEPAAFVDALLGX

SEQ ID NO: 70 - protein, 246 aa, Mycobacterium tuberculosis
  misc_feature (246)..(246): Xaa can be any naturally occurring amino acid
MYRVFEALDELSAIVEEARGVPMTAGCVVPRGDVLELIDDIKDAIPGELDDAQDVLDARDSMLQDAKTHADSMVSSATTEAESILNHARTEADRILSDAKAQADRMVSEARQHSERMVADAREEAIRIATAAKREYEASVSRAQAECDRLIENGNISYEKAVQEGIKEQQRLVSQNEVVAAANAESTRLVDTAHAEADRLRGECDIYVDNKLAEFEEFLNGTLRSVGRGRHQLRTAAGTHDYAVRX

SEQ ID NO: 71 - protein, 1877 aa, Mycobacterium tuberculosis
  misc_feature (1877)..(1877): Xaa can be any naturally occurring amino acid
MTGSISGEADLRHWLIDYLVTNIGCTPDEVDPDLSLADLGVSSRDAVVLSGELSELLGRTVSPIDFWEHPTINALAAYLAAPEPSPDSDAAVKRGARNSLDEPIAVVGMGCRFPGGISCPEALWDFLCERRSSISQVPPQRWQPFEGGPPEVAAALARTTRWGSFLPDIDAFDAEFFEISPSEADKMDPQQRLLLEVAWEALEHAGIPPGTLRRSATGVFAGACLSEYGAMASADLSQVDGWSNSGGAMSIIANRLSYFLDLRGPSVAVDTACSSSLVAIHLACQSLRTQDCHLAIAAGVNLLLSPAVFRGFDQVGALSPTGQCRAFDATADGFVRGEGAGVVVLKRLTDAQRDGDRVLAVICGSAVNQDGRSNGLMAPNPAAQMAVLRAAYTNAGMQPSEVDYVEAHGTGTLLGDPIEARALGTVLGRGRPEDSPLLIGSVKTNLGHTEAAAGIAGFIKTVLAVQHGQIPPNQHFETANPHIPFTDLRMKVVDTQTEWPATGHPRRAGVSSFGFGGTNAHVVIEQGQEVRPAPGQGLSPAVSTLVVAGKTMQRVSATAGMLADWMEGPGADVALADVAHTLNHHRSRQPKFGTVVARDRTQAIAGLRALAAGQHAPGVVNPADGSPGPGTVFVYSGRGSQWAGMGRQLLADEPAFAAAVAELEPVFVEQAGFSLHDVLANGEELVGIEQIQLGLIGMQLALTELWCSYGVRPDLVIGHSMGEVAAAVVAGALTPAEGLRVTATRSRLMAPLSGQGGMALLELDAPTTEALIADFPQVTLGIYNSPRQTVIAGPTEQIDELIARVRAQNRFASRVNIEVAPHNPAMDALQPAMRSELADLTPRTPTIGIISTTYADLHTQPVFDAEHWATNMRNPVRFQQAIASAGSGADGAYHTFIEISAHPLLTQAIIDTLHSAQPGARYTSLGTLQRDTDDVVTFRTNLNKAHTIHPPHTPHPPEPHPPIPTTPWQHTRHWITTKYPAGSVGSAPRAGTLLGQHTTVATVSASPPSHLWQARLAPDAKPYQGGHRFHQVEVVPASVVLHTILSAATELGYSALSEVRFEQPIFADRPRLIQVVADNRAISLASSPAAGTPSDRWTRHVTAQLSSSPSDSASSLNEHHRANGQPPERAHRDLIPDLAELLAMRGIDGLPFSWTVASWTQHSSNLTVAIDLPEALPEGSTGPLLDAAVHLAALSDVADSRLYVPASIEQISLGDVVTGPRSSVTLNRTAHDDDGITVDVTVAAHGEVPSLSMRSLRYRALDFGLDVGRAQPPASTGPVEAYCDATNFVHTIDWQPQTVPDATHPGAEQVTHPGPVAIIGDDGAALCETLEGAGYQPAVMSDGVSQARYVVYVADSDPAGADETDVDFAVRICTEITGLVRTLAERDADKPAALWILTRGVHESVAPSALRQSFLWGLAGVIAAEHPELWGGLVDLAINDDLGEFGPALAELLAKPSKSILVRRDGVVLAPALAPVRGEPARKSLQCRPDAAYLITGGLGALGLLMADWLADRGAHRLVLTGRTPLPPRRDWQLDTLDTELRRRIDAIRALEMRGVTVEAVAADVGCREDVQALLAARDRDGAAPIRGIIHAAGITNDQLVTSMTGDAVRQVMWPKIGGSQVLHDAFPPGSVDFFYLTASAAGIFGIPGQGSYAAANSYLDALARARRQQGCHTMSLDWVAWRGLGLAADAQLVSEELARMGSRDITPSEAFTAWEFVDGYDVAQAVVVPMPAPAGADGSGANAYLLPARNWSVMAATEVRSELEQGLRRIIAAELRVPEKELDTDRPFAELGLNSLMAMAIRREAEQFVGIELSATMLFNHPTVKSLASYLAKRVAPHDVSQDNQISALSSSAGSVLDSLFDRIESAPPEAERSVX

SEQ ID NO: 72 - protein, 1539 aa, Mycobacterium tuberculosis
  misc_feature (1539)..(1539): Xaa can be any naturally occurring amino acid
MMRTAFSRISGMTAQQRTSLADEFDRVSRIAVAEPVAVVGIGCRFPGDVDGPESFWDFLVAGRNAISTVPADRWDAEAFYHPDPLTPGRMTTKWGGFVPDVAGFDAEFFGITPREAAAMDPQQRMLLEVAWEALEHAGIPPDSLGGTRTAVMMGVYFNEYQSMLAASPQNVDAYSGTGNAHSITVGRISYLLGLRGPAVAVDTACSSSLVAVHLACQSLRLRETDLALAGGVSITLRPETQIAISAWGLLSPQGRCAAFDAAADGFVRGEGAGVVVLKRLTDAVRDGDQVLAVVRGSAVNQDGRSNGVTAPNTAAQCDVIADALRSGDVAPDSVNYVEAHGTGTVLGDPIEFEALAATYGHGGDACALGAVKTNIGHLEAAAGIAGFIKATLAVQRATIPPNLHFSQWNPAIDAASTRFFVPTQNSPWPTAEGPRRAAVSSFGLGGTNAHVIIEQGSELAPVSEGGEDTGVSTLVVTGKTAQRMAATAQVLADWMEGPGAEVAVADVAHTVNHHRARQATFGTVVARDRAQAIAGLRALAAGQHAPGVVSHQDGSPGPGTVFVYSGRGSQWAGMGRQLLADEPAFAAAVAELEPVFVEQAGFSLRDVIATGKELVGIEQIQLGLIGMQLTLTELWRSYGVQPDLVIGHSMGEVAAAVVAGALTPAEGLRVTATRARLMAPLSGQGGMALLGLDAAATEALIADYPQVTVGIYNSPRQTVIAGPTEQIDELIARVRAQNRFASRVNIEVAPHNPAMDALQPAMRSELADLTPRTPTIGIISTTYADLHTQPIFDAEHWATNMRNPVRFQQAIASAGSGADGAYHTFIEISAHPLLTQAIADTLEDAHRPTKSAAKYLSIGTLQRDADDTVTFRTNLYTADIAHPPHTCHPPEPHPTIPTTPWQHTHHWIATTHPSTAAPEDPGSNKVVVNGQSTSESRALEDWCHQLAWPIRPAVSADPPSTAAWLVVADNELCHELARAADSRVDSLSPPALAAGSDPAALLDALRGVDNVLYAPPVPGELLDIESAYQVFHATRRLAAAMVASSATAISPPKLFIMTRNAQPISEGDRANPGHAVLWGLGRSLALEHPEIWGGIIDLDDSMPAELAVRHVLTAAHGTDGEDQVVYRSGARHVPRLQRRTLPGKPVTLNADASQLVIGATGNIGPHLIRQLARMGAKTIVAMARKPGALDELTQCLAATGTDLIAVAADATDPAAMQTLFDRFGTELPPLEGIYLAAFAGRPALLSEMTDDDVTTMFRPKLDALALLHRRSLKSPVRHFVLFSSVSGLLGSRWLAHYTATSAFLDSFAGARRTMGLPATVVDWGLWKSLADVQKDATQISAESGLQPMADEVAIGALPLVMNPDAAVATVVVAADWPLLAAAYRTRGALRIVDDLLPAPEDVGKGESEFRTSLRSCPAEKRRDMLFDHVGALAATVMGMPPTEPLDPSAGFFQLGMDSLMSVTLQRALSESLGEFLPASVVFDYPTVYSLTDYLATVLPELLEIGATAVATQQATDSYHELTEAELLEQLSERLRGTQX

SEQ ID NO: 73 - protein, 2189 aa, Mycobacterium tuberculosis
  misc_feature (2189)..(2189): Xaa can be any naturally occurring amino acid
MTAATPDRRAIITEALHKIDDLTARLEIAEKSSSEPIAVIGMGCRFPGGVNNPEQFWDLLCAGRSGIVRVPAQRWDADAYYCDDHTVPGTICSTEGGFLTSWQPDEFDAEFFSISPREAAAMDPQQRLLIEVAWEALEDAGVPQHTIRGTQTSVFVGVTAYDYMLTLAGRLRPVDLDAYIPTGNSANFAAGRLAYILGARGPAVVIDTACSSSLVAVHLACQSLRGRESDMALVGGTNLLLSPGPSIACSRWGMLSPEGRCKTFDASADGYVRGEGAAVVVLKRLDDAVRDGNRILAVVRGSAVNQDGASSGVTVPNGPAQQALLAKALTSSKLTAADIDYVEAHGTGTPLGDPIELDSLSKVFSDRAGSDQLVIGSVKTNLGHLEAAAGVAGLMKAVLAVHNGYIPRHLNFHQLTPHASEAASRLRIAADGIDWPTTGRPRRAGVSSFGVSGTNAHVVIEQAPDPMAAAGTEPQRGPVPAVSTLVVFGKTAPRVAATASVLADWLDGPGAAVPLADVAHTLNHHRARQTRFGTVAAVDRRQAVIGLRALAAGQSAPGVVAPREGSIGGGTVFVYSGRGSQWAGMGRQLLADEPAFAAAIAELEPEFVAQGGFSLRDVIAGGKELVGIEQIQLGLIGMQLALTALWRSYGVTPDAVIGHSMGEVAAAVVAGALTPAQGLRVTAVRSRLMAPLSGQGTMALLELDAEATEALIADYPEVSLGIYASPRQTVISGPPLLIDELIDKVRQQNGFATRVNIEVAPHNPAMDALQPAMRSELADLTPQPPTIPIISTTYADLGISLGSGPRFDAEHWATNMRNPVRFHQAIAHAGADHHTFIEISAHPLLTHSISDTLRASYDVDNYLSIGTLQRDAHDTLEFHTNLNTTHTTHPPQTPHPPEPHPVLPTTPWQHTQHWITATSAAYHRPDTHPLLGVGVTDPTNGTRVWESELDPDLLWLADHVIDDLVVLPGAAYAEIALAAATDTFAVEQDQPWMISELDLRQMLHVTPGTVLVTTLTGDEQRCQVEIRTRSGSSGWTTHATATVARAEPLAPLDHEGQRREVTTADLEDQLDPDDLYQRLRGAGQQHGPAFQGIVGLAVTQAGVARAQVRLPASARTGSREFMLHPVMMDIALQTLGATRTATDLAGGQDARQGPSSNSALVVPVRFAGVHVYGDITRGVRAVGSLAAAGDRLVGEVVLTDANGQPLLVVDEVEMAVLGSGSGATELTNRLFMLEWEPAPLEKTAEATGALLLIGDPAAGDPLLPALQSSLRDRITDLELASAADEATLRAAISRTSWDGIVVVCPPRANDESMPDEAQLELARTRTLLVASVVETVTRMGARKSPRLWIVTRGAAQFDAGESVTLAQTGLRGIARVLTFEHSELNTTLVDIEPDGTGSLAALAEELLAGSEADEVALRDGQRYVNRLVPAPTTTSGDLAAEARHQVVNLDSSGASRAAVRLQIDQPGRLDALNVHEVKRGRPQGDQVEVRVVAAGLNFSDVLKAMGVYPGLDGAAPVIGGECVGYVTAIGDEVDGVEVGQRVIAFGPGTFGTHLGTIADLVVPIPDTLADNEAATFGVAYLTAWHSLCEVGRLSPGERVLIHSATGGVGMAAVSIAKMIGARIYTTAGSDAKREMLSRLGVEYVGDSRSVDFADEILELTDGYGVDVVLNSLAGEAIQRGVQILAPGGRFIELGKKDVYADASLGLAALAKSASFSVVDLDLNLKLQPARYRQLLQHILQHVADGKLEVLPVTAFSLHDAADAFRLMASGKHTGKIVISIPQHGSIEAIAAPPPLPLVSRDGGYLIVGGMGGLGFVVARWLAEQGAGLIVLNGRSAPSDEVAAAIAELNASGSRIEVITGDITEPDTAERLVRAVEDAGFRLAGVVHSAMVLADEIVLNMTDSAARRVFAPKVTGSWRLHVATAARDVDWWLTFSSAAALLGTPGQGAYAAANSWVDGLVAHRRSAGLPAVGINWGPWADVGRAQFFKDLGVEMINAEQGLAAMQAVLTADRGRTGVFSLDARQWFQSFPAVAGSSLFAKLHDSAARKSGQRRGGGAIRAQLDALDAAERPGHLASAIADEIRAVLRSGDPIDHHRPLETLGLDSLMGLELRNRLEASLGITLPVALVWAYPTISDLATALCERMDYATPAAAQEISDTEPELSDEEMDLLADLVDASELEAATRGESX

SEQ ID NO: 74 - protein, 1489 aa, Mycobacterium tuberculosis
  misc_feature (1489)..(1489): Xaa can be any naturally occurring amino acid
MSIPENAIAVVGMAGRFPGAKDVSAFWSNLRRGKESIVTLSEQELRDAGVSDKTLADPAYVRRAPLLDGIDEFDAGFFGFPPLAAQVLDPQHRLFLQCAWHALEDAGADPARFDGSIGVYGTSSPSGYLLHNLLSHRDPNAVLAEGLNFDQFSLFLQNDKDFLATRISHAFNLRGPSIAVQTACSSSLVAVHLACLSLLSGECDMALAGGSSLCIPHRVGYFTSPGSMVSAVGHCRPFDVRADGTVFGSGVGLVVLKPLAAAIDAGDRIHAVIRGSAINNDGSAKMGYAAPNPAAQADVIAEAHAVSGIDSSTVSYVECHGTGTPLGDPIEIQGLRAAFEVSQTSRSAPCVLGSVKSNIGHLEVAAGIAGLIKTILCLKNKALPATLHYTSPNPELRLDQSPFVVQSKYGPWECDGVRRAGVSSFGVGGTNAHVVLEEAPAEASEVSAHAEPAGPQVILLSAQTAAALGESRTALAAALETQDGPRLSDVAYTLARRRKHNVTMAAVVHDREHAATVLRAAEHDNVFVGEAAHDGEHGDRADAAPTSDRVVFLFPGQGAQHVGMAKGLYDTEPVFAQHFDTCAAGFRDETGIDLHAEVFDGTATDLERIDRSQPALFTVEYALAKLVDTFGVRAGAYIGYSTGEYIAATLAGVFDLQTAIKTVSLRARLMHESPPGAMVAVALGPDDVTQYLPPEVELSAVNDPGNCVVAGPKDQIRALRQRLTEAGIPVRRVRATHAFHTSAMDPMLGQFQEFLSRQQLRPPRTPLLSNLTGSWMSDQQVVDPASWTRQISSPIRFADELDVVLAAPSRILVEVGPGGSLTGSAMRHPKWSTTHRTVRLMRHPLQDVDDRDTFLRALGELWSAGVEVDWTPRRPAVPHLVSLPGYPFARQRHWVEPNHTVWAQAPGANNGSPAGTADGSTAATVDAARNGESQTEVTLQRIWSQCLGVSSVDRNANFFDLGGDSLMAISIAMAAANEGLTITPQDLYEYPTLASLTAAVDASFASSGLAKPPEAQANPAVPPNVTYFLDRGLRDTGRCRVPLILRLDPKIGLPDIRAVLTAVVNHHDALRLHLVGNDGIWEQHIAAPAEFTGLSNRSVPNGVAAGSPEERAAVLGILAELLEDQTDPNAPLAAVHIAAAHGGPHYLCLAIHAMVTDDSSRQILATDIVTAFGQRLAGEEITLEPVSTGWREWSLRCAALATHPAALDTRSYWIENSTKATLWLADALPNAHTAHPPRADELTKLSSTLSVEQTSELDDGRRRFRRSIQTILLAALGRTIAQTVGEGVVAVELEGEGRSVLRPDVDLRRTVGWFTTYYPVPLACATGLGALAQLDAVHNTLKSVPHYGIGYGLLRYVYAPTGRVLGAQRTPDIHFRYAGVIPELPSGDAPVQFDSDMTLPVREPIPGMGHAIELRVYRFGGSLHLDWWYDTRRIPAATAEALERTFPLALSALIQEAIAAEHTEHDDSEIVGEPEAGALVDLSSMDAGX

SEQ ID NO: 75 - protein, 332 aa, Mycobacterium tuberculosis
  misc_feature (332)..(332): Xaa can be any naturally occurring amino acid
MRNDDMAVVVNGVRKTYGKGKIVALDDVSFKVRRGEVIGLLGPNGAGKTTMVDILSTLTRPDAGSAIIAGYDVVSEPAGVRRSIMVTGQQVAVDDALSGEQNLVLFGRLWGLSKSAARKRAAELLEQFSLVHAGKRRVGTYSGGMRRRIDIACGLVVQPQVAFLDEPTTGLDPRSRQAIWDLVASFKKLGIATLLTTQYLEEADALSDRIILIDHGIIIAEGTANELKHRAGDTFCEIVPRDLKDLDAIVAALGSLLPEHHRAMLTPDSDRITMPAPDGIRMLVEAARRIDEARIELADIALRRPSLDHVFLAMTTDPTESLTHLVSGSARX

SEQ ID NO: 76 - protein, 327 aa, Mycobacterium tuberculosis
MTRSSNIPADATPNPHATAEQVAAARHDSKLAQVLYHDWEAENYDEKWSISYDQRCVDYARGRFDAIVPDEVIAQLPYDRALELGCGTGFFLLNLIQAGVARRGSVTDLSPGMVKVATRNGQALGLDIDGRVADAEGIPYDDDAFDLVVGHAVLHHIPDVELSLREVVRVLKPGGRFVFAGEPTTVGDGYARTLSTLTWRVVTNATKLPGLRGWRRPQGELDESSRAAALEALVDLHTFTPQDLQRIAHNAGAVEVQTATEEFTAAMLGWPLRTFECTVPPGRLGWGWARFAFTSWKTLGWVDANVWRHVVPKGWFYNVMITGVKPS

SEQ ID NO: 77 - protein, 525 aa, Mycobacterium tuberculosis
  misc_feature (525)..(525): Xaa can be any naturally occurring amino acid
MSIADTAAKPSTPSPANQPPVRTRAVIIGTGFSGLGMAIALQKQGVDFVILEKADDVGGTWRDNTYPGCACDIPSHLYSFSFEPKADWKHLFSYWDEILGYLKGVTDKYGLRRYIEFNSLVDRGYWDDDECRWHVFTADGREYVAQFLISGAGALHIPSFPEIAGRDEFAGPAFHSAQWDHSIDLTGKRVAIVGTGASAIQIVPEIVGQVAELQLYQRTPPWVVPRTNEELPVSLRRALRTVPGLRALLRLGIYWAQEALAYGMTKRPNTLKIIEAYAKYNIRRSVKDRELRRKLTPRYRIGCKRILNSSTYYPAVADPKTELITDRIDRITHDGIVTADGTGREVFREADVIVYATGFHVTDSYTYVQIKGRHGEDLVDRWNREGIGAHRGITVANMPNLFFLLGPNTGLGHNSVVFMIESQIHYVADAIAKCDRMGVQALAPTREAQDRFNQELQRRLAGSVWNSGGCRSWYLDEHGKNTVLWCGYTWQYWLTTRSVNPAEYRFFGIGNGLSSDRATVAAANX

SEQ ID NO: 78 - protein, 508 aa, Mycobacterium tuberculosis
  misc_feature (508)..(508): Xaa can be any naturally occurring amino acid
MLLHDVAITSMDVAATSSRLTKVARIAALLHRAAPDTQLVTIIVSWLSGELPQRHIGVGWAALRSLPPPAPQPALTVTGVDATLSKIGTLPGKGSQAQRAALVAELFSAATEAEQTFLLRLLGGELRQGAKGGIMADAVAQAAGLPAATVQRAAMLGGDLAAAAAAGLSGAALDTFTLRVGRPIGPMLAQTATSVHDALERHGGTTIFEAKLDGARVQIHRANDQVRIYTRSLDDVTARLPEVVEATLALPVRDLVADGEAIALCPDNRPQRFQVTASRFGRSVDVAAARATQPLSVFFFDILHRDGTDLLEAPTTERLAALDALVPARHRVDRLITSDPTDAANFLDATLAAGHEGVMAKAPAARYLAGRRGAGWLKVKPVHTLDLVVLAVEWGSGRRRGKLSNIHLGARDPATGGFVMVGKTFKGMTDAMLDWQTTRFHEIAVGPTDGYVVQLRPEQVVEVALDGVQRSSRYPGGLALRFARVVRYRADKDPAEADTIDAVRALYX

SEQ ID NO: 79 - protein, 308 aa, Mycobacterium tuberculosis
  misc_feature (308)..(308): Xaa can be any naturally occurring amino acid
MTSMYEQVDTNTADPVAGSRIDPVLARSWLLVNGAHGDRFESAAHSRADIVVLDIEDAVAPKDKHAARDNAVRWFGDGNADWVRINGFGTPWWADDLAMLADSPVGGVMLAMVESVDHVTETAKRLPNVPIVALVETARGLERINEIAAAKGTFRLAFGIGDFRRDTGFGEDPATLAYARSRFTIAARAAGLPSAIDGPTIGSNALKLIEATAVSAEFGMTGKICLSPDQCPVVNEGLSPSQDEIVWAKEFFAEFARDGGEIRNGSDLPRIARATKILDLARAYGIEVSDFEDEPVHMPAPTDTYHYX

SEQ ID NO: 80 - protein, 373 aa, Mycobacterium tuberculosis
  misc_feature (373)..(373): Xaa can be any naturally occurring amino acid
MSAHVATLHPEPPFALCGPRGTLIARGVRTRYCDVRAAQAALRSGTAPILLGALPFDVSRPAALMVPDGVLRARKLPDWPTGPLPKVRVAAALPPPADYLTRIGRARDLLAAFDGPLHKVVLARAVQLTADAPLDARVLLRRLVVADPTAYGYLVDLTSAGNDDTGAALVGASPELLVARSGNRVMCKPFAGSAPRAADPKLDAANAAALASSAKNRHEHQLVVDTMRVALEPLCEDLTIPAQPQLNRTAAVWHLCTAITGRLRNISTTAIDLALALHPTPAVGGVPTKAATELIAELEGDRGFYAGAVGWCDGRGDGHWVVSIRCAQLSADRRAALAHAGGGIVAESDPDDELEETTTKFATILTALGVEQX

SEQ ID NO: 81 - protein, 217 aa, Mycobacterium tuberculosis
  misc_feature (217)..(217): Xaa can be any naturally occurring amino acid
MADIDGVTGSAGLQPGPSEETDEELTARFERDAIPLLDQLYGGALRMTRNPADAEDLLQETMVKAYAGFRSFRHGTNLKAWLYRILTNTYINSYRKKQRQPAEYPTEQITDWQLASNAEHSSTGLRSAEVEALEALPDTEIKEALQALPEEFRMAVYYADVEGFPYKEIAEIMDTPIGTVMSRLHRGRRQLRGLLADVARDRGFARGEQAHEGVSSX

SEQ ID NO: 82 - protein, 765 aa, Mycobacterium tuberculosis
  misc_feature (765)..(765): Xaa can be any naturally occurring amino acid
MTIPRSQHMSTAVNSCTEAPASRSQWMLANLRHDVPASLVVFLVALPLSLGIAIASGAPIIAGVIAAVVGGIVAGAVGGSPVQVSGPAAGLTVVVAELIDELGWPMLCLMTIAAGALQIVFGLSRMARAALAIAPVVVHAMLAGIGITIALQQIHVLLGGTSHSSAWRNIVALPDGILHHELHEVIVGGTVIAILLMWSKLPAKVRIIPGPLVAIAGATVLALLPVLQTERIDLQGNFFDAIGLPKLAEMSPGGQPWSHEISAIALGVLTIALIASVESLLSAVGVDKLHHGPRTDFNREMVGQGSANVVSGLLGGLPITGVIVRSSANVAAGARTRMSTILHGVWILLFASLFTNLVELIPKAALAGLLIVIGAQLVKLAHIKLAWRTGNFVIYAITIVCVVFLNLLEGVAIGLVVAIVFLLVRVVRAPVEVKPVGGEQSKRWRVDIDGTLSFLLLPRLTTVLSKLPEGSEVTLNLNADYIDDSVSEAISDWRRAHETRGGVVAIVETSPAKLHHAHARPPKRHFASDPIGLVPWRSARGKDRGSASVLDRIDEYHRNGAAVLHPHIAGLTDSQDPYELFLTCADSRILPNVITASGPGDLYTVRNLGNLVPTDPDDRSVDAALDFAVNQLGVSSVVVCGHSSCAAMTALLEDDPANTTTPMMRWLENAHDSLVVFRNHHPARRSAESAGYPEADQLSIVNVAVQVERLTRHPILATAVAAADLQVIGIFFDISTARVYEVGPNGIICPDEPADRPVDHESAQX

SEQ ID NO: 83 - protein, 178 aa, Mycobacterium tuberculosis
  misc_feature (178)..(178): Xaa can be any naturally occurring amino acid
MGTCPCESSERNEPVSRVSGTNEVSDGNETNNPAEVSDGNETNNPAEVSDGNETNNPAPVSRVSGTNEVSDGNETNNPAPVSRVSGTNEVSDGNETNNPAPVTEKPLHPHEPHIEILRGQPTDQELAALIAVLGSISGSTPPAQPEPTRWGLPVDQLRYPVFSWQRITLQEMTHMRRX

SEQ ID NO: 84 - protein, 450 aa, Mycobacterium tuberculosis
  misc_feature (450)..(450): Xaa can be any naturally occurring amino acid
MAAVVKSVALAGRPTTPDRVHEVLGRSMLVDGLDIVLDLTRSGGSYLVDAITGRRYLDMFTFVASSALGMNPPALVDDREFHAELMQAALNKPSNSDVYSVAMARFVETFARVLGDPALPHLFFVEGGALAVENALKAAFDWKSRHNQAHGIDPALGTQVLHLRGAFHGRSGYTLSLTNTKPTITARFPKFDWPRIDAPYMRPGLDEPAMAALEAEALRQARAAFETRPHDIACFVAEPIQGEGGDRHFRPEFFAAMRELCDEFDALLIFDEVQTGCGLTGTAWAYQQLDVAPDIVAFGKKTQVCGVMAGRRVDEVADNVFAVPSRLNSTWGGNLTDMVRARRILEVIEAEGLFERAVQHGKYLRARLDELAADFPAVVLDPRGRGLMCAFSLPTTADRDELIRQLWQRAVIVLPAGADTVRFRPPLTVSTAEIDAAIAAVRSALPVVTX

SEQ ID NO: 85 - protein, 189 aa, Mycobacterium tuberculosis
  misc_feature (189)..(189): Xaa can be any naturally occurring amino acid
MTTRPATDRRKMPTGREEVAAAILQAATDLFAERGPAATSIRDIAARSKVNHGLVFRHFGTKDQLVGAVLDHLGTKLTRLLHSEAPADIIERALDRHGRVLARALLDGYPVGQLQQRFPNVAELLDAVRPRYDSDLGARLAVAHALALQFGWRLFAPMLRSATGIDELTGDELRLSVNDAVARILEPHX

SEQ ID NO: 86 - protein, 409 aa, Mycobacterium tuberculosis
  misc_feature (409)..(409): Xaa can be any naturally occurring amino acid
MKRFWENVGKPNDTTDGRGTTSLAMTPISQTPGLLAEAMVDLGAIEHNVRVLREHAGHAQLMAVVKADGYGHGATRVAQTALGAGAAELGVATVDEALALRADGITAPVLAWLHPPGIDFGPALLADVQVAVSSLRQLDELLHAVRRTGRTATVTVKVDTGLNRNGVGPAQFPAMLTALRQAMAEDAVRLRGLMSHMVYADKPDDSINDVQAQRFTAFLAQAREQGVRFEVAHLSNSSATMARPDLTFDLVRPGIAVYGLSPVPALGDMGLVPAMTVKCAVALVKSIRAGEGVSYGHTWIAPRDTNLALLPIGYADGVFRSLGGRLEVLINGRRCPGVGRICMDQFMVDLGPGPLDVAEGDEAILFGPGIRGEPTAQDWADLVGTIHYEVVTSPRGRITRTYREAENRX

SEQ ID NO: 87 - protein, 351 aa, Mycobacterium tuberculosis
  misc_feature (351)..(351): Xaa can be any naturally occurring amino acid
MAGSGVPSHRSMVIKVSVFAVVMLLVAAGLVVVFGDFRFGPTTVYHATFTDASRLKAGQKVRIAGVPVGSVKAVKLNPDHSIDVAFAIDRSYTLYSSTRAVIRYENLVGDRFLEITSGPGELRKLPPGGTINVAHTQPALDLDALLGGLRPVLKGFDADKINTITSAVIELLQGQGGPLANVLADTGAFSAALGARDQLIGEVITNLNAVLATVDAKSAQFSASVDQLQQLVSGLAKNRDPIAGAISPLASTTTDLTELLRNSRRPLQGILENARPLATELDNRKAEVNNDIEQLGEDYLRLSALGSYGAFFNIYFCSVTIKINGPAGSDILLPIGGQPDPSKGRCAFAKX

SEQ ID NO: 88 - protein, 1902 aa, Mycobacterium tuberculosis
  misc_feature (1902)..(1902): Xaa can be any naturally occurring amino acid
MSFVLIAPEFVTAAAGDLTNLGSSISAANASAASATTQVLAAGADEVSARIAALFGGFGLEYQAISAQVAAYHQRFVQALSTGAGAYASAEAAAAEQIVLGVINAPTQALLGRPLIGDGANATTPGGAGGAGGLLFGNGGAGAAGAPGQAGGPGGPAGLWGNGGPGGAGGSGGGTGGAGGAGGWLFGVGGAGGVGGAGGGTGGAGGPGGLIWGGGGAGGVGGAGGGTGGAGGRAELLFGAGGAGGAGTDGGPGATGGTGGHGGVGGDGGWLAPGGAGGAGGQGGAGGAGSDGGALGGTGGTGGTGGAGGAGGRGALLLGAGGQGGLGGAGGQGGTGGAGGDGVLGGVGGTGGKGGVGGVAGLGGAGGAAGQLFSAGGAAGAVGVGGTGGQGGAGGAGAAGADAPASTGLTGGTGFAGGAGGVGGQGGNAIAGGINGSGGAGGTGGQGGAGGMGGSGADNASGIGADGGAGGTGGNAGAGGAGGAAGTGGTGGVVGAAGKAGIGGTGGQGGAGGAGSAGTDATATGATGGTGFSGGAGGAGGAGGNTGVGGTNGSGGQGGTGGAGGAGGAGGVGADNPTGIGGTGGTGGKGGAGGAGGQGGSSGAGGTNGSGGAGGTGGQGGAGGAGGAGADNPTGIGGAGGTGGTGGAAGAGGAGGAIGTGGTGGAVGSVGNAGIGGTGGTGGVGGAGGAGAAAAAGSSATGGAGFAGGAGGEGGAGGNSGVGGTNGSGGAGGAGGKGGTGGAGGSGADNPTGAGFAGGAGGTGGAAGAGGAGGATGTGGTGGVVGATGSAGIGGAGGRGGDGGDGASGLGLGLSGFDGGQGGQGGAGGSAGAGGINGAGGAGGNGGDGGDGATGAAGLGDNGGVGGDGGAGGAAGNGGNAGVGLTAKAGDGGAAGNGGNGGAGGAGGAGDNNFNGGQGGAGGQGGQGGLGGASTTSINANGGAGGNGGTGGKGGAGGAGTLGVGGSGGTGGDGGDAGSGGGGGFGGAAGKAGGGGNGGRGGDGGDGASGLGLGLSGFDGGQGGQGGAGGSAGAGGINGAGGAGGNGGDGGDGATGAAGLGDNGGVGGDGGAGGAAGNGGNAGVGLTAKAGDGGAAGNGGNGGAGGAGGAGDNNFNGGQGGAGGQGGQGGLGGASTTSINANGGAGGNGGTGGKGGAGGAGTLGVGGSGGTGGDGGDAGSGGGGGFGGAAGKAGGGGNGGVGGDGGEGASGLGLGLSGFDGGQGGQGGAGGSAGAGGINGAGGAGGTGGAGGDGAPATLIGGPDGGDGGQGGIGGDGGNAGFGAGVPGDGGDGGNAGFGAGVPGDGGIGGTGGAGGAGGAGADGDPSIDGGQGGAGGHGGQGGKGGLNSTGLASAASGDGGNGGAGGAGGNGGDGDGFIGGSGGTGGTGGDAGVGGLANTGGTAGNAGIGGAGGRGGDGGAGDSGALSQDGNGFAGGQGGQGGVGGNAGAGGINGAGGTGGTGGAGGDGQNGTTGVASEGGAGGQGGDGGQGGIGGAGGNAGFGAGVPGDGGIGGTGGAGGAGGAGADGDPSIDGGQGGAGGHGGQGGKGGLNSTGLASAASGDGGNGGAGGAGGNGGDGDGFIGGSGGTGGTGGDAGVGGLANTGGTAGNAGIGGAGGRGGDGGAGDSGALSQDGNGFAGGQGGQGGVGGNAGAGGINGAGGTGGTGGAGGDGQNGTTGVASEGGAGGQGGDGGQGGIGGAGGNAGFGAGVPGDGGIGGTGGAGGAGGAGADGDPSIDGGQGGAGGHGGQGGKGGLNSTGLASAASGDGGNGGAGGAGGNGGAGGLGGGGGTGGTNGNGGLGGGGGNGGAGGAGGTPTGSGTEGTGGDGGDAGAGGNGGSATGVGNGGNGGDGGNGGDGGNGAPGGFGGGAGAGGLGGSGAGGGTDGDDGNGGSPGTDGSX

SEQ ID NO: 89 - protein, 1080 aa, Mycobacterium tuberculosis
  misc_feature (1080)..(1080): Xaa can be any naturally occurring amino acid
PQGADGNAGNGGDGGVGGNGGNGADNTTTAAAGTTGGAGGAGGAGGTGGTGGAAGTGTGGQQGNGGNGGNGGTGGKGGTGGDGALAGSSGGAGGKGGNGGDAGKAGTGSAPGTAGTGGDGGKGGNGGIGAAGTTGPVGTGASGGTGGSGGAGGTGGDGGAANGGTAGAGGAGGNGGKGGDGGAGVTSSTAGNSGGAGGSGGKGGDAGAGGAGATPGANGIAGNGGDGGDGAAGAVGISGATGAGDGGHGGTGAAGGNGGTGGAGGSGIDGVGGGTGGTGGNGGNGAIGGAGGDAGGSGNSGGNGGIGGKGGNAGAGGAAGSNGGTVGANGTGGDGGNGGAAGAATAGSNGGAGTGSAGGNGGTGGRGGSGGAGGDGIGGVGGGKGGNGADGEVGGAGGAGGSGPNTSPGGNGGQGGQGGSGGAGGAAGAGGAGGGANGTAGNGGQGGAGGTGGAGAASSATNGGSGGAGGTGGDGGSGGAGGTGGAGGTGGAAGDGGQGGQGGAGGGAGGQGGAGGAGGTGGNGGNITGGTAGTAGAAGNGGAAGKGGAGGQGGTGGGTGGQGGAGGDGGAGGTGGDRTVGGGTVPAGSGGQGGNAGGGGAGGQGGADGGSGGDGGDAGTGGNGGNGGNRNSGNGTGGAGGNGGGGANGGAGGAGGSGGGTGGNGGAGGDAGDAGNGGNGNGTGNGGNGGNGGIAGMGGNGGAGTGSGNGGNGGSGGNGGNAGMGGNSGTGSGDGGAGGNGGAAGTGGTGGDGGLTGTGGTGGSGGTGGDGGNGGNGADNTANMTAQAGGDGGNGGDGGFGGGAGAGGGGLTAGANGTGGQGGAGGDGGNGAIGGHGPLTDDPGGNGGTGGNGGTGGTGGAGIGSLGGGTGGDGGNGGNGGTGGEGGEVGGAGGTGGAAGNGGDGGTGGTGGGDGGAGGTGGTGGTGGLGDPRVGGSGGDGGTGGSGGAAGNGGNGGNAGAGGNGNGGTGGAGGIGGTGGNGGDAEPGVPPGAGGAGGAGTTGGKGGTGGNGSGTGSGGTGGDGGTGGGGGNGGTGWNGGKGDTGSGGGAGDGGKAPAGGTGGAGGDGGAGGKGGSGGVX

SEQ ID NO: 90 - protein, 377 aa, Mycobacterium tuberculosis
MDLNFDDETLAFQAEVREFLAANAASIPTKSYDNAEGFAQHRYWDRVLFDAGLSVITWPAKYGGRDAPLLHWIVFEEEYFRAGAPGRASANGTSMLAPTLFAHGTAEQLDRILPKMASGEQIWAQAWSEPESGSDLASLRSTASKVDGGWLLNGQKIWSSRAPFADMGFGLFRSDPAVERHRGLTYFMFDLKAKGVTVRPIAQLGGDTGFGEIFLDDVFVPDRDVIGAPNDGWRAAMSTSSNERGMSLRSPARFLASAERLVQLWKDRGSPPEFADRVADAWIKAQAYRLQTFGTVTRLAAGGELGAESSVTKVFWSELDVHLHQTALDLRGADGELAGPWTEGLLFALGGPIYAGTNEIQRNIIAERLLGLPREKT

SEQ ID NO: 91 - protein, 359 aa, Mycobacterium tuberculosis
  misc_feature (359)..(359): Xaa can be any naturally occurring amino acid
MHAVTRPTLREAVARLAPGTGLRDGLERILRGRTGALIVLGHDENVEAICDGGFSLDVRYAATRLRELCKMDGAVVLSTDGSRIVRANVQLVPDPSIPTDESGTRHRSAERAAIQTGYPVISVSHSMNIVTVYVRGERHVLTDSATILSRANQAIATLERYKTRLDEVSRQLSRAEIEDFVTLRDVMTVVQRLELVRRIGLVIDYDVVELGTDGRQLRLQLDELLGGNDTARELIVRDYHANPEPPSTGQINATLDELDALSDGDLLDFTALAKVFGYPTTTEAQDSTLSPRGYRAMAGIPRLQFAHADLLVRAFGTLQGLLAASAGDLQSVDGIGAMWARHVREGLSQLAESTISDQX

SEQ ID NO: 92 - protein, 288 aa, Mycobacterium tuberculosis
  misc_feature (288)..(288): Xaa can be any naturally occurring amino acid
MTVSDSPAQRQTPPQTPGGTAPRARTAAFFDLDKTIIAKSSTLAFSKPFFAQGLLNRRAVLKSSYAQFIFLLSGADHDQMDRMRTHLTNMCAGWDVAQVRSIVNETLHDIVTPLVFAEAADLIAAHKLCGRDVVVVSASGEEIVGPIARALGATHAMATRMIVEDGKYTGEVAFYCYGEGKAQAIRELAASEGYPLEHCYAYSDSITDLPMLEAVGHASVVNPDRGLRKEASVRGWPVLSFSRPVSLRDRIPAPSAAAIATTAAVGISALAAGAVTYALLRRFAFQPX

SEQ ID NO: 93 - protein, 329 aa, Mycobacterium tuberculosis
  misc_feature (329)..(329): Xaa can be any naturally occurring amino acid
MTIMRAVVAESSDRLVWQEVPDVSAGPGEVLIKVAASGVNRADVLQAAGKYPPPPGVSDIIGLEVSGIVAAVGPGVTEWSAGQEVCALLAGGGYAEYVAVPADQVLPIPPSVNLVDSAALPEVACTVWSNLVMTAHLRPGQLVLIHGGASGIGSHAIQVVRALAARVAITAGSPEKLELCRDLGAQITINYRDEDFVARLKQETDGSGADIILDIMGASYLDRNIDALATDGQLIVIGMQGGVKAELNLGKLLTKRARVIGTTLRARPVSGPHGKAAIAQAVAASVWPMIAANRVRPVIGTRLPIQQAAQAHELMLSGKTFGKILLTVX

SEQ ID NO: 94 - protein, 644 aa, Mycobacterium tuberculosis
  misc_feature (644)..(644): Xaa can be any naturally occurring amino acid
MPSRRKSPQFGHEMGAFTSARAREVLVALGQLAAAVVVAVGVAVVSLLAIARVEWPAFPSSNQLHALTTVGQVGCLAGLVGIGWLWRHGRFRRLARLGGLVLVSAFTVVTLGMPLGATKLYLFGISVDQQFRTEYLTRLTDTAALRDMTYIGLPPFYPPGWFWIGGRAAALTGTPAWEMFKPWAITSMAIAVAVALVLWWRMIRFEYALLVTVATAAVMLAYSSPEPYAAMITVLLPPMLVLTWSGLGARDRQGWAAVVGAGVFLGFAATWYTLLVAYGAFTVVLMALLLAGSRLQSGIKAAVDPLCRLAVVGAIAAAIGSTTWLPYLLRAARDPVSDTGSAQHYLPADGAALTFPMLQFSLLGAICLLGTLWLVMRARSSAPAGALAIGVLAVYLWSLLSMLATLARTTLLSFRLQPTLSVLLVAAGAFGFVEAVQALGKRGRGVIPMAAAIGLAGAIAFSQDIPDVLRPDLTIAYTDTDGYGQRGDRRPPGSEKYYPAIDAAIRRVTGKRRDRTVVLTADYSFLSYYPYWGFQGLTPHYANPLAQFDKRATQIDSWSGLSTADEFIAALDKLPWQPPTVFLMRHGAHNSYTLRLAQDVYPNQPNVRRYTVDLRTALFADPRFVVEDIGPFVLAIRKPQESAX

SEQ ID NO: 95 - protein, 1095 aa, Mycobacterium tuberculosis
  misc_feature (1095)..(1095): Xaa can be any naturally occurring amino acid
MPHDGNERSHRIARLAAVVSGIAGLLLCGIVPLLPVNQTTATIFWPQGSTADGNITQITAPLVSGAPRALDISIPCSAIATLPANGGLVLSTLPAGGVDTGKAGLFVRANQDTVVVAFRDSVAAVAARSTIAAGGCSALHIWADTGGAGADFMGIPGGAGTLPPEKKPQVGGIFTDLKVGAQPGLSARVDIDTRFITTPGALKKAVMLLGVLAVLVAMVGLAALDRLSRGRTLRDWLTRYRPRVRVGFASRLADAAVIATLLLWHVIGATSSDDGYLLTVARVAPKAGYVANYYRYFGTTEAPFDWYTSVLAQLAAVSTAGVWMRLPATLAGIACWLIVSRFVLRRLGPGPGGLASNRVAVFTAGAVFLSAWLPFNNGLRPEPLIALGVLVTWVLVERSIALGRLAPAAVAIIVATLTATLAPQGLIALAPLLTGARAIAQRIRRRRATDGLLAPLAVLAAALSLITVVVFRDQTLATVAESARIKYKVGPTIAWYQDFLRYYFLTVESNVEGSMSRRFAVLVLLFCLFGVLFVLLRRGRVAGLASGPAWRLIGTTAVGLLLLTFTPTKWAVQFGAFAGLAGVLGAVTAFTFARIGLHSRRNLTLYVTALLFVLAWATSGINGWFYVGNYGVPWYDIQPVIASHPVTSMFLTLSILTGLLAAWYHFRMDYAGHTEVKDNRRNRILASTPLLVVAVIMVAGEVGSMAKAAVFRYPLYTTAKANLTALSTGLSSCAMADDVLAEPDPNAGMLQPVPGQAFGPDGPLGGISPVGFKPEGVGEDLKSDPVVSKPGLVNSDASPNKPNAAITDSAGTAGGKGPVGINGSHAALPFGLDPARTPVMGSYGENNLAATATSAWYQLPPRSPDRPLVVVSAAGAIWSYKEDGDFIYGQSLKLQWGVTGPDGRIQPLGQVFPIDIGPQPAWRNLRFPLAWAPPEADVARIVAYDPNLSPEQWFAFTPPRVPVLESLQRLIGSATPVLMDIATAANFPCQRPFSEHLGIAELPQYRILPDHKQTAASSNLWQSSSTGGPFLFTQALLRTSTIATYLRGDWYRDWGSVEQYHRLVPADQAPDAVVEEGVITVPGWGRPGPIRALPX

SEQ ID NO: 96 - protein, 517 aa, Mycobacterium tuberculosis
  misc_feature (517)..(517): Xaa can be any naturally occurring amino acid
MQVTSVGHAGFLIQTQAGSILCDPWVNPAYFASWFPFPDNSGLDWGALGECDYLYVSHLHKDHFDAENLRAHVNKDAVVLLPDFPVPDLRNELQKLGFHRFFETTDSVKHRLRGPNGDLDVMIIALRAPADGPIGDSALVVADGETTAFNMNDARPVDLDVLASEFGHIDVHMLQYSGAIWYPMVYDMPARAKDAFGAQKRQRQMDRARQYIAQVGATWVVPSAGPPCFLAPELRHLNDDGSDPANIFPDQMVFLDQMRAHGQDGGLLMIPGSTADFTGTTLNSLRHPLPAEQVEAIFTTDKAAYIADYADRMAPVLAAQKAGWAAAAGEPLLQPLRTLFEPIMLQSNEICDGIGYPVELAIGPETIVLDFPKRAVREPIPDERFRYGFAIAPELVRTVLRDNEPDWVNTIFLSTRFRAWRVGGYNEYLYTFFKCLTDERIAYADGWFAEAHDDSSSITLNGWEIQRRCPHLKADLSKFGVVEGNTLTCNLHGWQWRLDDGRCLTARGHQLRSSRPX

SEQ ID NO: 97 - protein, 748 aa, Mycobacterium tuberculosis
  misc_feature (748)..(748): Xaa can be any naturally occurring amino acid
MTTKKFTPTITRGPRLTPGEISLTPPDDLGIDIPPSGVQKILPYVMGGAMLGMIAIMVAGGTRQLSPYMLMMPLMMIVMMVGGLAGSTGGGGKKVPEINADRKEYLRYLAGLRTRVTSSATSQVAFFSYHAPHPEDLLSIVGTQRQWSRPANADFYAATRIGIGDQPAVDRLLKPAVGGELAAASAAPQPFLEPVSHMWVVKFLRTHGLIHDCPKLLQLRTFPTIAIGGDLAGAAGLMTAMICHLAVFHPPDLLQIRVLTEEPDDPDWSWLKWLPHVQHQTETDAAGSTRLIFTRQEGLSDLAARGPHAPDSLPGGPYVVVVDLTGGKAGFPPDGRAGVTVITLGNHRGSAYRIRVHEDGTADDRLPNQSFRQVTSVTDRMSPQQASRIARKLAGWSITGTILDKTSRVQKKVATDWHQLVGAQSVEEITPSRWRMYTDTDRDRLKIPFGHELKTGNVMYLDIKEGAEFGAGPHGMLIGTTGSGKSEFLRTLILSLVAMTHPDQVNLLLTDFKGGSTFLGMEKLPHTAAVVTNMAEEAELVSRMGEVLTGELDRRQSILRQAGMKVGAAGALSGVAEYEKYRERGADLPPLPTLFVVVDEFAELLQSHPDFIGLFDRICRVGRSLRVHLLLATQSLQTGGVRIDKLEPNLTYRIALRTTSSHESKAVIGTPEAQYITNKESGVGFLRVGMEDPVKFSTFYISGPYMPPAAGVETNGEAGGPGQQTTRQAARIHRFTAAPVLEEAPTPX

SEQ ID NO: 98 - protein, 407 aa, Mycobacterium tuberculosis
  misc_feature (407)..(407): Xaa can be any naturally occurring amino acid
MPSPRREDGDALRCGDRSAAVTEIRAALTALGMLDHQEEDLTTGRNVALELFDAQLDQAVRAFQQHRGLLVDGIVGEATYRALKEASYRLGARTLYHQFGAPLYGDDVATLQARLQDLGFYTGLVDGHFGLQTHNALMSYQREYGLAADGICGPETLRSLYFLSSRVSGGSPHAIREEELVRSSGPKLSGKRIIIDPGRGGVDHGLIAQGPAGPISEADLLWDLASRLEGRMAAIGMETHLSRPTNRSPSDAERAATANAVGADLMISLRCETQTSLAANGVASFHFGNSHGSVSTIGRNLADFIQREVVARTGLRDCRVHGRTWDLLRLTRMPTVQVDIGYITNPHDRGMLVSTQTRDAIAEGILAAVKRLYLLGKNDRPTGTFTFAELLAHELSVERAGRLGGSX

SEQ ID NO: 99 - DNA, 20 bp, Artificial sequence
  Chemically synthesized
aatatctggtccgcttgcac

SEQ ID NO: 100 - DNA, 20 bp, Artificial sequence
  Chemically synthesized
acacgatctcgtcgctaacc

SEQ ID NO: 101 - DNA, 20 bp, Artificial sequence
  Chemically synthesized
atcttcggccacataacctg

SEQ ID NO: 102 - DNA, 20 bp, Artificial sequence
  Chemically synthesized
gagaggctgtctacccgaga

SEQ ID NO: 103 - DNA, 20 bp, Artificial sequence
  Chemically synthesized
gagaaggtcagccaccagtc

SEQ ID NO: 104 - DNA, 20 bp, Artificial sequence
  Chemically synthesized
atggtcgagaaagtcgatgg

SEQ ID NO: 105 - DNA, 20 bp, Artificial sequence
  Chemically synthesized
tttccaccgcttctatcgac

SEQ ID NO: 106 - DNA, 20 bp, Artificial sequence
  Chemically synthesized
ggtgatgtccatctctttgg

SEQ ID NO: 107 - DNA, 20 bp, Artificial sequence
  Chemically synthesized
tccggatctgtaccgaaatc

SEQ ID NO: 108 - DNA, 20 bp, Artificial sequence
  Chemically synthesized
attcgcctaagtcgtcgttg

SEQ ID NO: 109 - DNA, 20 bp, Artificial sequence
  Chemically synthesized
gactacccgcaggtgacagt

SEQ ID NO: 110 - DNA, 20 bp, Artificial sequence
  Chemically synthesized
gtggagatgattccgatggt

SEQ ID NO: 111 - DNA, 20 bp, Artificial sequence
  Chemically synthesized
cctcgacctgaatctcaagc

SEQ ID NO: 112 - DNA, 20 bp, Artificial sequence
  Chemically synthesized
atcttgccggtgtgtttacc

SEQ ID NO: 113 - DNA, 20 bp, Artificial sequence
  Chemically synthesized
gacatcgaactcgacgacaa

SEQ ID NO: 114 - DNA, 20 bp, Artificial sequence
  Chemically synthesized
cggtggaaaagtcaagcact

SEQ ID NO: 115 - DNA, 20 bp, Artificial sequence
  Chemically synthesized
agaggaccctaacggcaaat

SEQ ID NO: 116 - DNA, 20 bp, Artificial sequence
  Chemically synthesized
tttccaccgcttctatcgac

SEQ ID NO: 117 - DNA, 20 bp, Artificial sequence
  Chemically synthesized
ctacgctacgtggtggattc

SEQ ID NO: 118 - DNA, 20 bp, Artificial sequence
  Chemically synthesized
ggtgatgtccatctctttgg

(19) United States
(12) Patent Application Publication
(10) Pub. No.: US 2014/0349320 A1
(43) Pub. Date: Nov. 27, 2014

(54) Using Adaptive Immunity to Detect Drug Resistance

(71) Applicant: The Trustees of the University of Pennsylvania, Philadelphia, PA (US)

(72) Inventors: Gregory P. Bisson, Philadelphia, PA (US); Drew Weissman, Wynnewood, PA (US); Harvey Rubin, Philadelphia, PA (US)

(21) Appl. No.: 14/365,479

(22) PCT Filed: Dec. 17, 2012
(86) PCT No.: PCT/US12/70092

§ 371 Date: Jun. 13, 2014

Related U.S. Application Data
(60) Provisional application No. 61/576,182, filed on Dec. 15, 2011.

(51) Int. Cl. G01N 33/569

(52) U.S. Cl. CPC G01N 33/5695; G01N 2800/44
USPC 435/7.92; 530/350

(57) ABSTRACT

The present invention relates to a method of using adaptive immunity to detect drug resistance in infectious diseases. The invention provides novel antigens associated with drug resistant MTB infection.

### CROSS-REFERENCE TO RELATED APPLICATIONS

This application claims priority to U.S. Provisional Patent Application No. 61/576,182, filed on Dec. 15, 2011, which is hereby incorporated herein by reference in its entirety.

### BACKGROUND OF THE INVENTION

Mycobacterium tuberculosis (MTB) is a transmissible human pathogen that can cause either latent (asymptomatic) infection or active TB disease (TB). Nearly one third of the world's population is estimated to be infected with MTB (including both latent and active infections), and while progress is being made in terms of control of drug-susceptible TB (Lonnroth et al., 2010 Lancet 375:1814-29), 9.4 million cases of TB were identified and treated world-wide in 2008 (WHO Global tuberculosis control: short update to 2009 report; In: Organization WH, editor. Geneva, Switzerland, 2009). Despite this progress, drug resistance in MTB, and particularly multi-drug resistant TB (MDR-TB, defined as TB due to MTB that is resistant to the first line drugs isoniazid and rifampicin) has emerged as a threat to control of TB both in the US and abroad (Gandhi et al., 2010, Lancet 375(9728):1830-43). Global surveillance systems for drug-resistance in TB are inadequate, and therefore data are insufficient to inform whether the incidence of drug-resistant/MDR-TB TB is rising or falling. However, of the estimated 440,000 cases of MDR-TB occurring in 2008, only 7% were identified and reported to WHO and of these only a fifth were treated according to WHO standards (WHO. Global tuberculosis control: short update to 2009 report. In: Organization WH, editor. Geneva, Switzerland, 2009), suggesting that the problem is under-recognized and its true scope is unknown. The Global Project on Anti-Tuberculosis Drug Resistance, reporting on data collected from 83 countries, found the prevalence of drug resistance in new cases of active TB to be approximately 11% (Wright et al., 2009, Lancet 2009 373(9678):1861-73). Furthermore, mathematical modeling suggests that population control of drug-sensitive TB disease does not imply control of drug-resistant strains. Rather, removal of patients with drug-susceptible disease from a population may replenish the pool of individuals who are fully susceptible to MTB infection with minority circulating drug-resistant strains (Cohen et al., 2004, Nat Med 10(10):1117-21). While MDR-TB appears under control in some countries, in other settings rates of MDR-TB among all cases of TB are alarming. For example, in two provinces in China and in nine countries of the former Soviet Union, >7% of all new cases of active TB disease were MDR-TB (Wright et al., 2009, Lancet 373(9678):1861-73). Although drug sensitive active TB disease can be cured in 6 months, treatment of MDR-TB requires use of costly, toxic, and often ineffective second-line antimicrobials for >24 months (Shah et al., 2007, Emerge Infect Dis 13(3):380-7), is associated with high rates of morbidity and mortality, and has been described as both an international public health emergency (WHO. Multi-drug and extensively drug-resistant TB (M/XDR-TB): 2010 global report on surveillance and response, 2010) and a threat to the goal of TB elimination in the US.

Drug resistance in MTB is due primarily to single nucleotide polymorphisms in genes encoding key mycobacterial enzymes (Blanchard, 1996, Annu Rev Biochem 65:215-39). The rpoB gene encodes the β-subunit of bacterial RNA polymerase, which is the target of rifampicin (Campbell et al., 2001, Cell 104:901-12; Jin and Gross, 1988, J Mol Biol 202:45-58). Mutations in this gene account for over 95% of clinical cases of rifampicin resistance (Telenti et al., 1993, Lancet 341:647-50) and are commonly associated with the presence of MDR-TB (Geffen, 2010. Cepheid Gene Xpert diagnostic technology for TB. HTB South; Shah et al., 2007, Emerg Infect Dis 13:380-7). Although some rifampicin resistant strains demonstrate mild in vitro fitness losses compared to wild-type parent strains, the most common MTB clinical strains containing the most frequent rpoB mutations (e.g., such as the S531L mutation) tend to exhibit little or no fitness defects, suggesting that certain MTB isolates are capable of overcoming, at least to some extent, initial fitness deficiencies associated with antibiotic resistance (Gagneux, 2009, Clin Microbiol Infect 15 Suppl 1:66-8; Gagneux et al., 2006, Science 312:1944-6). Bergval et al. reported a two to five-fold induction of the stress response gene dnaE2 (but not recA) in four of six rpoB mutants of MTB when compared to their wild-type isogenic parent strains using RT-PCR (Bergval et al., 2007, FEMS Microbiol Lett 275:338-43). However, relatively little is known about MTB adaptive mechanisms to drug resistance, which may compensate for mutations in rpoB. Recently, compensatory mutations in RNA polymerase genes in rpoB-mutant isolates of MTB have been identified by comparative genomics (Comas et al., 2011, Nat Genet 44:106-10).

Specific gene upregulation associated with rpoB mutation has been observed in numerous model organisms including Streptomyces spp, which are environmental organisms phylogenetically related to MTB (Hu et al., 2002, J Bacteriol 184:3984-91; Inaoka et al., 2004, J Biol Chem 279:3885-92; Tala et al., 2009, J Bacteriol 191:805-14). Moreover, upregulation of certain gene clusters via rpoB mutation has been used in actinomycetes as a way to discover new secondary metabolites, including antibiotics (Hosaka et al., 2009, Nat Biotechnol 27:462-4). This suggests that rpoB mutation may have analogous effects on specific gene upregulation in MTB. The MTB genome has an extensive array of polyketide synthase genes (Cole, S et al., 1998, Nature 393:537-44), which have been shown in other bacteria to be involved in the biosynthesis of secondary metabolites, including rifamycins (Gokhale et al., 2007, Nat Prod Rep 24:267-77). rpoB is an essential gene in MTB and clinically relevant rpoB mutations occur near the DNA-RNA channel of bacterial RNA polymerase (Campbell et al., 2001, Cell 104:901-12; Sassetti et al., 2003, Mol Microbiol 48:77-84).

Evaluation of drug resistance in MTB can be performed using culture with drug susceptibility testing (DST), via molecular tests, or via microscopy (Pai et al., 2006, Expert Rev Mol Diagn 6(3):423-32). These approaches all require access to the bacterium in clinical specimens such as sputum and are useful when these specimens can be obtained. However, such approaches are useless for detecting drug resistance when clinical samples containing the pathogen are difficult or impossible to collect. This situation arises in several types of active TB disease including “sputum-scarce” TB disease, extrapulmonary TB, and TB in children. Dependence on acquisition of clinical specimens for culture or molecular testing is also a problem in latent MTB infection because specimens containing the pathogen cannot be obtained during this stage of infection. Rather, diagnosis of latent MTB infection relies on detection of host memory cellular immune responses directed at MTB antigens. Examples of this approach include the 100-year old tuberculin skin test (TST) and a newer “in-tube” interferon gamma (IFN-γ) release assays, which are now recommended by the US Center for Disease Control and Prevention (CDC) for diagnosis of latent MTB infection (Pai et al., 2007, Lancet Infect Dis 7(6):428-38). However, these tests do not provide information about whether the infection being detected is due to a drug-resistant strain. Because of this limitation, there is currently no way to define the epidemiology of or identify individuals with drug-resistant latent MTB infection. This is of public health importance because standard treatment of latent MTB infection relies on drugs that do not work in drug resistant TB and treatment of latent MTB infection is a cornerstone of TB eradication efforts.

Thus, there is an urgent need in the art for new compositions and methods for determining whether MTB infection is due to a drug-resistance strain. The present invention addresses this need.

### SUMMARY OF THE INVENTION

The invention provides a method of detecting an immune response against any antigen that is expressed differentially between a drug-resistant strain compared to a corresponding drug-susceptible strain for the diagnosis of an infection with a drug resistant strain.

In one embodiment, the invention provides a method of assessing microbial infection in a mammal comprising: a) contacting at least one polypeptide that is differentially expressed in a drug-resistant strain compared to a corresponding drug-susceptible strain with a biological sample from the mammal; and b) measuring an immune response in the biological sample wherein an increase in the immune response to the at least one polypeptide that is differentially expressed in a drug-resistant strain compared to a corresponding drug-susceptible strain with a biological sample from the mammal indicates a past or present microbial infection with a drug-resistant strain.

In one embodiment, the microbial infection is M. tuberculosis infection.

In one embodiment, the at least one polypeptide that is differentially expressed in a drug-resistant strain compared to a corresponding drug-susceptible strain is selected from the group consisting of SEQ ID NOs: 1-98 or a fragment thereof.

In one embodiment, the immune response is at least one selected from the group consisting of a cell-mediated response and a humoral response.

In one embodiment, the biological sample comprises at least one selected from the group consisting of whole blood, serum, plasma, and peripheral blood mononuclear cells.

In one embodiment, the cell-mediated response is a T cell response.

In one embodiment, the T cell response is measured by detecting expression of a marker for T cell activation or proliferation.

In another embodiment, the T cell response is measured by detecting secretion of a cytokine.

In one embodiment, the humoral response is measured by a) contacting the biological sample of the mammal with the at least one polypeptide that is differentially expressed in a drug-resistant strain compared to a corresponding drug-susceptible strain for a sufficient amount of time to allow complex formation between the at least one polypeptide with an antibody present in the biological sample, and b) detecting a complex formed between an antibody in the biological sample and the antigen, wherein detection of the complex is indicative of infection by the drug-resistant strain in the mammal.

In one embodiment, the mammal is a human.

In one embodiment, the mammal is receiving or has previously received a therapeutic intervention.

The invention also provides a kit for use in determining the presence of a drug-resistant strain in a mammal. In one embodiment, the kit comprises at least one polypeptide that is differentially expressed in a drug-resistant strain compared to a corresponding drug-susceptible strain.

In one embodiment, the drug-resistant strain is M. tuberculosis.

In one embodiment, the at least one polypeptide that is differentially expressed in a drug-resistant strain compared to a corresponding drug-susceptible strain is selected from the group consisting of SEQ ID NOs: 1-98 or a fragment thereof.

In one embodiment, the at least one polypeptide that is differentially expressed in a drug-resistant strain compared to a corresponding drug-susceptible strain is used to detect an immune response in a biological sample from a mammal.

In one embodiment, the biological sample comprises at least one selected from the group consisting of whole blood, serum, plasma, and peripheral blood mononuclear cells.

In one embodiment, the immune response is at least one selected from the group consisting of a cell-mediated response and a humoral response.

In one embodiment, the cell-mediated response is measured by detecting expression of a marker for T cell activation or proliferation.

In one embodiment, the cell-mediated response is measured by detecting secretion of a cytokine.

In one embodiment, the humoral response is detected by detecting a complex formed between an antibody in the biological sample and the at least one polypeptide that is differentially expressed in a drug-resistant strain compared to a corresponding drug-susceptible strain.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an image depicting a thin layer chromatography of lipid extracts obtained from M. tuberculosis clinical isolates in triplicate from independent cultures. rpoB mutants and wild type isolates for each genotype are shown. A PDIM standard is also shown.

FIG. 2 depicts a heat map of protein detection in rpoB mutant vs. wild-type MTB as determined by summary cell-associated spectral counts in 4 gene clusters detected as possibly involved in natural product biosynthesis by the program NPsearcher. The phthioceral dimycoserosate (PDIM) biosynthetic locus is Cluster 4. The other shown clusters are as follows: Cluster 1=Rv0993-Rv1017c; Cluster 2=Rv1536-Rv1551; Cluster 3=Rv1661-Rv1668c; Cluster 4=Rv2930-Rv2943. Genes are shown in increasing order of Rv number from top to bottom. The column on the left corresponds to the Haarlem strains, while the one on the right corresponds to the Beijing strains. This figure is useful for understanding the finding that genes preferentially expressed by drug-resistant MTB isolates are involved in secondary metabolism.

FIG. 3, comprising FIGS. 3A through 3D, is a series of images demonstrating the effect of a common rpoB mutation, S450L, on MTB gene expression and fitness. FIG. 3A depicts the expression of the PDIM biosynthesis locus in the rpoB-mutant Beijing strain relative to the rifampin-sensitive wild-type parent strain during logarithmic growth in nutrient-rich broth, as measured by RT-PCR. FIG. 3B depicts gene expression of the same strains after 72 h of infection in activated murine macrophages. FIG. 3C depicts growth and survival of the rpoB mutant (grey) relative to the Beijing wild-type parent strain (white) in activated murine macrophages. FIG. 3D depicts RT-PCR analysis of gene expression in a laboratory-generated RpoB S450L mutant relative to the isogenic CDC1551 wild-type strain during logarithmic growth in nutrient-rich broth. The cycle threshold value (CT) obtained for each gene of interest was normalized with that of the housekeeping gene sigA. Fold regulation of individual genes was calculated using the following formula: 2-(C(CT)-S(CT)), where C represents the wild-type (control) strain and S represents the rpoB mutant strain. This figure is useful for demonstrating that certain genes are expressed by rpoB mutant MTB differentially in macrophages, which is a natural environment of MTB during human infection.

FIG. 4 is an image depicting a representative spectra for some of the significant features found at higher abundance in the chloroform fraction of rpoB mutant vs. wild-type susceptible MTB isolates, which also matched peaks found in the PDIM standard. Retention time for these peaks was 11.32 min. The main differentially significant peaks are shown by an arrow (m/z 797.7 and m/z 823.2). On the figure, CI22=Beijing rpoB mutant.

### DETAILED DESCRIPTION

The present invention provides compositions and methods for identifying a gene and encoded protein thereof that is associated with drug resistance in a microbial strain (referred elsewhere herein as a drug resistant gene and encoded protein thereof from a drug-resistant microbial strain). In one embodiment, the method includes identifying a gene in an infectious microbe that is in the process of undergoing transformation from drug sensitivity to drug resistance. In another embodiment, the method includes identifying a gene in an infectious microbe that has already undergone transformation from a drug sensitive to drug resistant state.

In one embodiment, the invention relates to a method of identifying a gene in a microbe wherein the expression of the gene is modulated when the microbe is drug resistant as compared to the expression of the gene in an otherwise identical microbe not induced to become drug resistant. The identified gene can be isolated and accordingly, the invention includes variants, derivatives or fragments of the isolated gene and protein and peptide products, variants and derivatives encoded thereby. In one embodiment, the drug resistant protein of the invention comprises the amino acid sequence of one or more of the sequences of SEQ ID NOs: 1-98. Also encompassed by the invention are antibodies or cellular immune responses that bind to or target the drug resistance proteins of the invention.

In one embodiment, the invention relates to the comparison of the proteomes and metabolomes of paired wild-type and rpoB-mutant MTB clinical isolates and the identification of compensatory mechanisms important to drug-resistant isolates of this pathogen. In one embodiment, the products identified at significantly higher spectral counts in the cell wall fraction of a rpoB-mutant, rifampicin resistant strain represent proteins that are transcriptionally coupled on a 50-kb region involved in the biosynthesis of PDIM in MTB, including but not limited to two type-I polyketide synthase genes (Rv2933/ppsC and Rv2935/ppsE) and a probable daunorubicin imycoserosate (DIM) transport protein (Rv2936/drrA). Other proteins identified in other cellular fractions besides the cell wall include a succinate semialdehyde dehydrogenase (Rv0234c), a putative integration host factor (Rv1388/mihF), a probable acyl-coA dehydrogenase (Rv3562/fadE31) involved in lipid degradation and a polynucleotide phosphorylase/polyadenylase (Rv2783c/gpsI) involved in mRNA degradation. In addition, three products identified as (Rv1056, Rv3038c, and Rv3661) which are conserved hypotheticals of unknown function, are also included in the invention as examples of proteins expressed at higher levels (relative to wild-type isolates).

Following the identification of a drug-resistant protein following the methods of the invention, the protein and fragments thereof can be used to diagnose whether a mammal, preferably a human, is infected with a drug-resistant form of an infectious microbe. Preferably, the drug-resistant form of an infectious microbe is MTB.

The present invention is based on the concept that the adaptive immune response of a mammal can be used to determine the presence or absence of drug resistance because the immune response is capable of responding to an antigen associated with drug resistance in a microbe.

In some instances, the method for identifying current infection by a drug resistant microbe in a mammal relies on detecting a cell-mediated or humoral memory immune response associated with a drug resistant protein of the invention. In other instances, detection of the memory immune response to proteins expressed during drug resistance allows for the identification of a prior drug resistant infection in the mammal.

In one embodiment, the invention includes a method of using immune responses against a drug resistant protein or otherwise a protein associated with drug resistance of the invention comprising the amino acid sequence of one or more of the sequences of SEQ ID NOs: 1-98 in an assay to detect microbial infection in a mammal. In one embodiment, the method of detecting a microbial infection in a mammal includes isolating whole blood or peripheral blood mononuclear cells (PBMCs) from a mammal and exposing the whole blood or PBMCs to one or more drug resistant proteins of the invention to determine the presence or absence of an immune response against the drug resistant protein. Detection of an immune response against the drug resistant protein is an indication that the mammal is now or has been previously infected with a drug resistant microbe. Preferably, the immune response against the drug resistant protein is a cell-mediated or humoral immune response.

In one embodiment, the invention includes an assay for measuring the magnitude of cellular immunologic responses to proteins upregulated by a drug resistant microbe (e.g., rpoB-mutant, rifampicin resistant MTB and other drug resistance mutations including those associated with aminoglycosides (amikacin, kanamycin), polypeptides (capreomycin, viomycin, enviomycin), Fluoroquinolones (ciprofloxacin, levofloxacin, moxifloxacin), thioamides (ethionamide, prothionamide), rifabutin, macrolides (clarithromycin), linezolid, thioacetazone, thioridazine, arginine, vitamin D, R207910, and other new drugs being developed). In some instances, blood is drawn from a patient and the blood is incubated with one or more of the proteins upregulated by the drug resistant microbe. After a period of incubation time, the amount of cytokine is measured to determine the extent of the immune response directed to the one or more of the proteins upregulated by the drug resistant microbe, wherein detection of an immune response indicates that the patient is infected with a drug resistant microbe.

The invention provides the use of any assay to assess for an immunological response against any antigen that is expressed differentially between a drug-resistant strain compared to a corresponding drug-susceptible strain.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antigen” or “Ag” as used herein is defined as a molecule that provokes an immune response, including both B and T cell responses. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response can encode an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences can be arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “analyte” as used herein may be a cell lysate or extract, a tissue lysate or extract, a cell culture supernatant, a tissue culture supernatant, or a body fluid. In some instances, “analyte” refers to a proteome or a mixture of different proteomes.

As used herein, the term “assessing” includes; diagnosing mycobacterial infection manifesting as TB; diagnosing drug resistant latent mycobacterial infection which does not manifest as disease; differentiating between active and latent mycobacterial infection; and monitoring the progress or change in the status of mycobacterial infection over time. In some instances, the change may occur spontaneously or as a result of treatment with a drug or vaccine or a test drug or test vaccine.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e g, amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The term “epitope” as used herein is defined as the part of an antigen that elicits an immune response, inducing B and/or T cell responses. An antigen can have one or more epitopes. Most antigens have many epitopes; i.e., they are multivalent. In general, an epitope is roughly 8 to 11 amino acids and/or sugars in length (for MHC Class I epitopes) or longer (for MHC Class II epitopes). One skilled in the art understands that generally the overall three-dimensional structure or the linear sequence of the molecule can be the main criterion of antigenic specificity distinguishing one epitope from another.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the phrase “infection and/or exposure” includes such diverse conditions in a patient as mycobacterial infection resulting in active disease, clearance, or latency, response to anti mycobacterial vaccination, close contact with an individual having or suspected of having a mycobacterial infection or with a mycobacterium that causes disease in humans, including in immunocompromised humans.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Naturally occurring” as used herein describes a composition that can be found in nature as distinct from being artificially produced. For example, a nucleotide sequence present in an organism, which can be isolated from a source in nature and which has not been intentionally modified by a person in the laboratory, is naturally occurring.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “phenotypically distinct” is used to describe organisms, tissues, cells or components thereof, which can be distinguished by one or more characteristics, observable and/or detectable by current technologies. Each of such characteristics may also be defined as a parameter contributing to the definition of the phenotype. Wherein a phenotype is defined by one or more parameters an organism that does not conform to one or more of the parameters shall be defined to be distinct or distinguishable from organisms of the phenotype.

The terms “patient,” “subject,” “individual,” and the like are used interchangeably herein, and refer to any animal, or cells thereof whether in vitro or in situ, amenable to the methods described herein. Preferably, the patient, subject or individual is a mammal, and more preferable, a human.

The term “proteome” as used herein refers to the specific protein composition of a cell, tissue or organism. Depending on the individual cells contained therein, a culture of a cell or a tissue could, theoretically, contain as many proteomes as there are cells contained therein. For convenience, the proteomes of one cell culture or one tissue is regarded as representing one proteome. The proteomes of one type of organism may differ from another depending on the status and genomic background of its cells.

A “reference level” of a marker means a level of the marker that is indicative of a particular disease state, phenotype, or lack thereof, as well as combinations of disease states, phenotypes, or lack thereof. A “positive” reference level of a marker means a level that is indicative of a particular disease state or phenotype. A “negative” reference level of a marker means a level that is indicative of a lack of a particular disease state or phenotype.

As used herein, the term “tuberculosis” comprises disease states usually associated with infections caused by mycobacteria species comprising the M. tuberculosis complex, including M. africanum, M. bovis, M. bovis BCG, M. microti, M. canetti, M. pinnipedii, and M. mungi.

The term “mycobacterial infection” is also associated with mycobacterial infections caused by mycobacteria other than M. tuberculosis (MOTT), including M. avium-intracellulare, M. kansasii, M. fortuitum, M. chelonae, M. leprae, M. avium subspecies paratuberculosis, M. intracellulare, M. scrofulaceum, M. xenopi, M. abscessus, M. marinum, and M. ulcerans.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing, decreasing the future risk of, or removing all signs of the disease or disorder. As another example, administration of the agent after clinical manifestation of the disease to combat the symptoms of the disease comprises “treatment” of the disease.

A “therapeutic” treatment is a treatment administered to a subject who exhibits symptoms or signs of disease or pathology, for the purpose of diminishing or eliminating those signs.

As used herein, “treating a disease or disorder” means reducing the frequency with which a symptom of the disease or disorder is experienced by a patient.

“Variant” as the term is used herein, is a nucleic acid sequence or a peptide sequence that differs in sequence from a reference nucleic acid sequence or peptide sequence respectively. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

For sequence comparison, typically one sequence acts as a reference sequence, to which test sequences may be compared. When using a sequence comparison algorithm, test and reference sequences are input into a computer, subsequent coordinates are designated, if necessary, and sequence algorithm program parameters are designated. The sequence comparison algorithm then calculates the percentage sequence identity for the test sequence(s) relative to the reference sequence, based on the designated program parameters.

As used herein, “vaccination” is intended for prophylactic or therapeutic vaccination.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1, 2, 2.7, 3, 4, 5, 5.3, and 6. This applies regardless of the breadth of the range.

### DESCRIPTION

Infection with a microbe, such as mycobacteria (e.g. MTB), leads to an active disease state in a minority of individuals. Most individuals who become infected with MTB develop latent infection, which can be cleared by the host or which may persist for many years. In some individuals with latent MTB infection, MTB reactivates to cause active disease. The invention provides a strategy for controlling drug-resistant infection with MTB, whether latent or active, by detection of drug resistance in the microbe using adaptive immune responses to antigens expressed during the drug resistant state.

The present invention is based on the discovery that differentially expressed genes and encoded proteins thereof (e.g., a candidate antigen) between a drug-susceptible MTB strain and a corresponding drug-resistant MTB strain can be readily identified. Accordingly, the invention provides compositions and methods for identifying a novel drug-resistant antigen and, thereby, a drug resistant infection.

The present invention also relates to a comprehensive comparison of proteomes and metabolomes between rpoB mutant, rifampicin resistant MTB with those of their paired wild-type, rifampicin susceptible parent strains which identified several genes, particularly those involved in secondary metabolism such as genes involved in the biosynthesis of cell wall lipids including phthiocerol dimycocerosate (PDIM), that are upregulated in rpoB mutants, both in broth culture and, in particular, when grown in murine macrophages. In one embodiment, the products identified at significantly higher spectral counts in the cell wall fraction of a rpoB mutant, rifampicin-resistant strain represent proteins that are transcriptionally coupled on a 50-kb region involved in the biosynthesis of phthiocerol dimycocerosate (PDIM) in Mtb, including two type-I polyketide synthase genes (including Rv2933/ppsC and Rv2935/ppsE) and a probable daunorubicin imycoserosate (DIM) transport protein (Rv2936/drrA). Other proteins identified in other cellular fractions besides the cell wall include a succinate semialdehyde dehydrogenase (Rv0234c), a putative integration host factor (Rv1388/mihF), a probable acyl-coA dehydrogenase (Rv3562/fadE31) involved in lipid degradation and a polynucleotide phosphorylase/polyadenylase (Rv2783c/gpsI) involved in mRNA degradation. In addition, three products identified as (Rv1056, Rv3038c, and Rv3661) which are conserved hypotheticals of unknown function, are also included in the invention as examples of a drug-resistant protein.

The invention includes a method of determining whether a drug-resistant mutant strain derived from a drug-susceptible strain expresses different genes and encoded proteins compared to the drug-susceptible strain. In one embodiment, the method identifies a gene and encoded protein that is present in the mutant drug-resistant strain but is absent in corresponding wild-type drug-susceptible strains. In another embodiment, the invention provides a method of distinguishing between a drug-susceptible and a drug-resistant strain of mycobacteria.

When armed with the drug-resistant antigen of the invention, the antigen can be used to diagnose, treat and prevent mycobacterial drug-resistant infection, whether latent or active. In one embodiment, the gene and encoded protein associated with drug-resistance in mycobacteria of the invention can be used for diagnosing, treating and preventing mycobacterial drug-resistant infection (latent or active) in a mammal. Preferably, the mammal is human.

In one embodiment, the invention includes a method of using the immune response against a protein that is differentially expressed in a drug resistant strain compared to a corresponding wild type strain. The differentially expressed protein in the drug resistant strain is considered to be a drug resistant antigen of the invention that can be assayed for the presence thereof in a biological sample from a mammal to detect microbial infection in the mammal. In one embodiment, the invention includes a method of using an immune response against an antigen of the invention comprising the amino acid sequence of one or more of the sequences of SEQ ID NOs: 1-98 in an assay to detect microbial infection in a mammal. In one embodiment, the method of detecting a microbial infection in a mammal includes isolating whole blood or specifically peripheral blood mononuclear cells (PBMCs) from a mammal and exposing the blood or PBMCs to one or more drug resistant proteins of the invention to determine the presence or absence of an immune response against the drug resistant protein. Detection of an immune response against the drug resistant protein is an indication that the mammal is now or has been previously infected with a drug resistant microbe. Preferably, the immune response against the drug resistant protein is a cell-mediated or humoral immune response.

In one embodiment, the invention includes an assay for measuring the magnitude of cellular immunologic responses to proteins upregulated by a drug resistant microbe (e.g., rpoB mutant, rifampicin resistant MTB but can include resistance to any antibiotic used to treat TB infection). In some instances, blood is drawn from a patient and the blood is incubated with one or more of the proteins, peptide pools, select antigenic peptides, or other methods used to deliver a protein for immunologic presentation, upregulated by the drug resistant microbe (i.e., a drug-resistant antigen of the invention). After a period of incubation time, the amount of cytokine or the number of antigen-specific PBMCs secreting one or more cytokines is measured to determine the extent of the immune response directed to the one or more of the proteins upregulated by the drug resistant microbe, wherein detection of an immune response indicates that the patient is infected with a drug resistant microbe.

In one embodiment, one or more of the genes and encoded proteins associated with drug-resistance in mycobacteria of the invention can be used as an immunological composition. For example, the protein or otherwise antigen of the present invention can be used as a vaccine for targeting drug-resistant mycobacteria in a mammal. Another example is use of the protein or otherwise antigen of the present invention to detect immune responses in a mammal, in order to determine if the mammal's infection is drug resistant.

### Identifying a Drug-Resistant Gene and Encoded Protein

In order to diagnose, treat and prevent mycobacterial drug-resistant infection, whether active or latent in a mammal, it is desirable to first identify a drug-resistant antigen. Accordingly, the invention includes compositions and methods for determining whether a drug-resistant progeny strain expresses different genes and encoded proteins compared to that of a corresponding drug-susceptible strain.

Accordingly, the invention contemplates the identification of differentially expressed markers by whole genome nucleic acid microarray, to identify markers differentially expressed between a drug-resistant progeny strain and a corresponding drug-susceptible strain. The invention further contemplates using methods known to those skilled in the art to detect and to measure the level of differentially expressed marker expression products, such as RNA and protein, to measure the level of one or more differentially expressed marker expression products.

Typical diagnostic methods focusing on nucleic acids include amplification techniques such as PCR and RT-PCR (including quantitative variants), and hybridization techniques such as in situ hybridization, microarrays, blots, and others. Typical diagnostic methods focusing on proteins include binding techniques such as ELISA, imunohistochemistry, microarray and functional techniques such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

In one embodiment, the method comprises screening for a drug-resistance gene and encoded protein from a MTB strain that is resistant to rifampicin (RFP, 3-(4-Methyl-1-piperazinyliminolmethyl)-rifamycin). However, the invention is not limited to rifampicin. That is, rifampicin is merely used as a non-limiting example. Therefore, any antitubercular drug resistance is applicable to the present invention. The method comprises culturing MTB isolates (e.g., a drug-susceptible parent isolate and a drug-resistance progeny isolate) under the same conditions with drug or without drug and comparing differentially expressed genes and encoded proteins. Preferably, the proteome of each cell lysate from each MTB isolate culture is compared to identify differentially expressed proteins. For example, a comparison of the in vitro proteome of the cell lysates derived from a drug-susceptible strain of MTB and a laboratory-derived rifampicin-resistant mutant strain derived from the parent strain cultured in the presence and absence of rifampicin can be obtained using a standard preoteomic analysis methodology.

In one embodiment, the invention provides a method of screening for a drug-resistant protein in a microbe (e.g., MTB) using proteomic technology. Proteomic techniques allow for the representation of the proteome characteristics of the microbe in a high-throughput way. The proteome characteristics of each microbe can be compared with each other to generate a comparative proteome profile. The comparative proteome profile can be used to determine differentially expressed proteins between the microbial strains. For example, using comparative proteome analysis, a protein in an infectious microbe that is undergoing or has undergone transformation from drug sensitivity to drug resistance can be identified.

The invention also includes a method for mass spectrometry analysis for determining the proteome profile for the desired microbe. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation.

Mass spectrometers are commonly combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the invention. In such combinations, “MS” can refer to any form of mass spectrometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

It is often necessary to prepare samples comprising an analyte of interest for MS. Such preparations include without limitation purification and/or buffer exchange. Any appropriate method, or combination of methods, can be used to prepare samples for MS. One preferred type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., glycoconjugates, proteins, peptides, and nucleic acids, and, with mobile phase supplements, oligonucleotides). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions. However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HPLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, trifluoroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity. Where the complexity of the mixture is very high, it is possible to couple ion exchange chromatography and RP-HPLC in tandem to identify proteins from mixtures containing in excess of 1,000 proteins.

A particular type of MS technique, matrix-assisted laser desorption time-of-flight mass spectrometry (MALDI-TOF MS) can also be used to analyze biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method is used for detection and characterization of biomolecules, such as proteins, peptides, oligosaccharides and oligonucleotides, with molecular masses between about 400 and about 500,000 Da, or higher. MALDI-MS is a sensitive technique that allows the detection of low quantities of analyte in a sample.

Partial amino acid sequences of proteins can be determined by enzymatic proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences can be used in vitro as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (2001 Neuroscientist 7:12).

Tryptic peptides can be directly analyzed using MALDI-TOF. However, where sample complexity is apparent, on-line or off-line LC-MS/MS or two-dimensional LC-MS/MS may be necessary to separate the peptides. For example, for simple digests, a gradient of 5-45% (v/v) acetonitrile in 0.1% formic acid (or TFA, if MALDI MS/MS is available) over 45 min, and then 45-95% acetonitrile in 0.1% formic acid (or TFA, if MALDI MS/MS is available) over 5 min can be used. 0.1% Formic acid solution is used on the Q-TOF instrument and 0.1% TFA solution is used on the Dionex Probot fraction collector for off-line coupling between HPLC and MALDI-MS/MS analysis (carried out on the ABI 4700). For a complex sample, a gradient of 5-45% (v/v) acetonitrile over 90 min, and then 45-95% acetonitrile over 30 min can be used. For a very complex sample, a gradient of 5-45% (v/v) acetonitrile over 120 min, and then 45-95% acetonitrile over 60 min might be used. On the Q-TOF, one survey scan and four MS/MS data channels are used to acquire CID data with 1.4 s scan time.

In another embodiment, the invention comprises any method known in the art to effectively detect an antigen, an antibody, or an antigen-antibody complex in a sample. Suitable methods include, but are not limited to, immunoassays, enzyme assays, mass spectrometry, biosensors, and chromatography. Thus, the invention includes the use of any type of instrumentality to detect a desired antigen, antibody, or antigen-antibody complex in a sample.

In one embodiment, an immunoassay can be an enzyme-linked immunosorbant immunoassay (ELISA), a sandwich assay, a competitive assay, a radioimmunoassay (RIA), a lateral flow immunoassay, a Western Blot, an immunoassay using a biosensor, an immunoprecipitation assay, an agglutination assay, a turbidity assay or a nephelometric assay.

### Compositions for Diagnosis and Treatment

Following the identification of the drug-resistant antigen using the methods of the invention, the antigen may be used to diagnose, treat and prevent mycobacterial drug-resistant infection, whether active or latent.

In one embodiment, the invention provides compositions comprising a unique protein or proteins or otherwise an antigen or group of antigens preferentially expressed by a drug-resistant infected cell. In general, the antigen or antigens of the present invention are recognized by the host's adaptive immune system. Preferably, the antigen is recognized by a cell-mediated immune response, although the invention also includes approaches based on the humoral immune system as well. In one embodiment, the antigen of the present invention is a polypeptide having an amino acid sequence selected from the group consisting of SEQ ID NOs: 1-98, or any combination thereof.

In another aspect, the present invention provides a method for using the antigen or antigens of the present invention to detect drug resistance in a pathogenic microbe or infection of a pathogenic microbe. Preferably, the microbe is MTB. In one embodiment, the method includes detecting MTB exposure or infection in a test biological sample using an antigen or antigens of the invention or fragment(s) thereof. Other known MTB antigens may be used in combination with the antigens of the invention.

Cellular immune-based assays have been developed and are used in the clinical setting. Measurement of cell-mediated immune responses is important for immune diagnosis of many infectious and autoimmune diseases, as a marker for detection of T-cell responses to an antigen. Current methods for detecting cell-mediated immune responses include skin tests measuring both immediate and delayed type hypersensitivity, lymphocyte proliferation assays and measurement of cytokines produced by purified mononuclear cells cultured with antigen. Most in vitro methods for detecting cell-mediated immune responses involve the purification of lymphocytes from whole blood, culturing these lymphocytes with an antigen for periods from 12 hours to 6 days and then detecting T-cell reactivity to the antigen. Established methods, such as the proliferation assay, use the uptake of radioactive isotopes by dividing T-cells as a marker for cell mediated immune response reactivity. More recently, techniques such as a single cell assay (ELISpot) have been used to detect the number of T-cells producing certain cytokines in response to the antigenic stimulation.

The present invention provides a method for measuring cell-mediated immune responses in a subject by incubating a sample from the subject which comprises T-cells or other cells of the immune system with an antigen. Production of IFN-gamma or other cytokine or immune effector molecule(s) is then detected. The presence or level of immune effector is then indicative of the level of cell mediated responsiveness of the subject.

The present invention provides an assay of the potential or capacity of a subject to mount a cell-mediated response. The assay is based on measuring immune effector molecule production by cells of the immune system in response to antigenic stimulation. The immune effectors may be detected using ligands such as antibodies specific for the effectors or by measuring the level of expression of genes encoding the effectors. The present invention provides, therefore, a means to determine the responsiveness of cell mediated immune response in a subject and, in turn, provides a means for the diagnosis of infectious diseases, pathological conditions, level of immunocompetence and a marker of T-cell responsiveness to endogenous or exogenous antigens.

Accordingly, one aspect of the present invention contemplates a method for measuring a cell mediated immune response in a subject. The method comprises collecting a sample from the subject wherein the sample comprises cells of the immune system which are capable of producing immune effector molecules following stimulation by an antigen, incubating the sample with an antigen and then measuring the presence of or elevation in the level of an immune effector molecule wherein the presence or level of the immune effector molecule is indicative of the capacity of the subject to mount a cell-mediated immune response.

In one embodiment, the present invention provides a method for measuring a cell mediated immune response in a human subject, the method comprising collecting a sample from the human subject wherein the sample comprises cells of the immune system which are capable of producing immune effector molecules following stimulation by an antigen, incubating the sample with an antigen and then measuring the presence of or elevation in the level of an immune effector molecule wherein the presence or level of said immune effector molecule is indicative of the capacity of the human subject to mount a cell-mediated immune response.

The immune effector molecules may be any of a range of molecules which are produced in response to cell activation or stimulation by an antigen. Although an interferon such as IFN-γ is a particularly useful immune effector molecule, others include a range of cytokines such as interleukins (IL), e.g. IL-2, IL-4, IL-10 or IL-12, tumor necrosis factor alpha (TNF-alpha), a colony stimulating factor (CSF) such as granulocyte (G)-CSF or granulocyte macrophage (GM)-CSF amongst many others including chemokines, proteins associated with degranulation (CD107a), and proteins associated with lysis (granzyme, perforin).

Accordingly, in another preferred embodiment, the present invention provides a method for measuring a cell-mediated immune response in a subject, said method comprising collecting a sample from the subject wherein the sample comprises cells of the immune system which are capable of producing IFN-gamma molecules following stimulation by an antigen, incubating the sample with an antigen and then measuring the presence of or elevation in the level of an IFN-gamma molecule wherein the presence or level of the IFN-gamma molecule is indicative of the capacity of said subject to mount a cell-mediated immune response.

As far as the preferred embodiment extends to humans is concerned, the present invention further provides a method for measuring a cell mediated immune response in a human subject, the method comprising collecting a sample from the subject wherein the sample comprises cells of the immune system which are capable of producing IFN-gamma molecules following stimulation by an antigen, incubating the sample with an antigen and then measuring the presence or elevation in level of an IFN-gamma molecule wherein the presence or level of said IFN-gamma molecule is indicative of the capacity of said human subject to mount a cell-mediated immune response.

Some of these assays measure ex vivo IFN-γ T cell responses from blood samples after overnight incubation with desired protein antigens which are present in M. tuberculosis. Broadly, two commercially available assay formats exist: enzyme-linked immunospot (ELISpot) and enzyme-linked immunoassay (ELISA). These assays are believed to be a significant advancement in the diagnosis of MTB infection. Accordingly, the antigens of the present invention can be added to similar assays in order to not only diagnose previous or current MTB infection (which present assays can do), but also detect any antitubercular drug resistance, such as rifampicin resistance in the infection as well. However, the invention is not limited to these assays, but rather, the protein antigens of the present invention can be used in any assay in the art for immunologically detecting an infectious agent that has developed drug resistance.

The present invention provides assays for detecting drug resistance in MTB using adaptive immune responses. The assays of the invention can be integrated into existing “in-tube” cellular assays, for example, simply by adding the drug-resistance antigen(s) of the present invention in an additional tube. Because these “in-tube” assays have been shown to be cost effective in resource limited settings (Burgos et al., 2009, Int J Tuberc Lung Dis 13(8):962-8), the assays of the present invention relating to diagnosing drug resistance can translate into improved clinical care.

In one embodiment, the present invention provides an improvement to the existing cellular immune-based assays for identifying mycobacterial infection. This is because the antigens of the present invention are useful for identifying drug resistant infection as opposed to simply detecting infection.

In addition to cell-mediated assays, the antigens of the present invention can be used in a variety of assay formats known to those of ordinary skill in the art for using polypeptides to detect antibodies in a sample or using an antigen binding agent to detect polypeptides in a sample. See, e.g., Harlow and Lane, Antibodies: A Laboratory Manual, Cold Spring Harbor Laboratory, 2001. In one example, the assay involves the use of one or more polypeptides of SEQ ID NOs: 1-98 immobilized on a solid support to bind to and remove the antibody or polypeptide from the sample. The bound polypeptide or antibody (i.e., the formation of a polypeptide-antibody) may then be detected using a detection reagent that contains a reporter group. Suitable detection reagents include, but are not limited to antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the polypeptide or polypeptide binding agent of the invention may be attached. For example, the solid support may be a test well in a microtiter plate or a nitrocellulose membrane or other suitable membranes. Alternatively, the support may be a bead or disc, such as, but not limited to, glass, fiberglass, latex or a plastic material such as polystyrene or polyvinylchloride. The support may also be a magnetic particle or a fiber optic sensor.

The polypeptide or polypeptide binding agent of the invention may be bound to the solid support using a variety of techniques known to those of ordinary skill in the art, which are amply described in the patent and scientific literature. In the context of the present technology, the term “bound” refers to both noncovalent association, such as adsorption, and covalent attachment (which may be a direct linkage between the polypeptide or the antigen-binding agent and functional groups on the support or may be a linkage by way of a cross-linking agent). The polypeptides or polypeptide binding agents of the invention may also be bound by adsorption to a well in a microtiter plate or to a membrane. In such cases, adsorption may be achieved by contacting the polypeptides or polypeptide binding agents of the invention, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide or polypeptide binding agent ranging from about 10 ng to about 1 mg, and about 100 mg, is sufficient to bind an adequate amount of polypeptide or antibody.

In certain embodiments, the assay is an enzyme immunoassay (EIA) or enzyme-linked immunosorbent assay (ELISA). In these assays, an enzyme, which is bound to the polypeptide or antigen-binding agent will react with an appropriate substrate, e.g., a chromogenic substrate, in such a manner as to produce a chemical moiety which can be detected, for example, by spectrophotometric, fluorimetric or by visual means.

In some embodiments, in an EIA or ELISA, the assay is performed by first contacting a polypeptide antigen that has been immobilized on a solid support, commonly the well of a microtiter plate, with a test sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo., USA) may be employed. The immobilized polypeptide is then incubated with the sample, and antibody is allowed to bind to the polypeptide. The sample may be diluted with a suitable diluent, such as phosphate-buffered saline (PBS) prior to incubation. In some embodiments, an appropriate contact time (i.e., incubation time) is that period of time that is sufficient to detect the presence of antibody within a test biological sample. In some embodiments, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% (v/v) Tween 20™. Detection reagent may then be added to the solid support. An appropriate detection reagent is any compound that binds to the immobilized antibody-polypeptide complex and that can be detected by any of a variety of means known to those in the art. Suitable detection reagents include, but are not limited to binding agents such as, Protein A, Protein C, immunoglobulin, lectin or free antigen conjugated to a reporter group. Suitable reported groups include, but are not limited to, e.g., enzymes (such as horseradish peroxidase and alkaline phosphatase), substrates, cofactors, inhibitors, dyes, radionuclides, luminescent groups, fluorescent groups, biotin and colloidal particles, such as colloidal gold and selenium. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., USA, and Pierce, Rockford, Ill., USA).

Enzymes which can be used to detectably label the polypeptide-antibody complex formed include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

All known variants of ELISA type assays may be used in the methods of the present technology, including but not limited to, e.g., indirect ELISA, sandwich EISA, competitive ELISA (see e.g., U.S. Pat. Nos. 5,908,781 and 7,393,843). Additionally other ELISA methods known in the art may be used in the methods of the present technology.

Other assays for use in the methods of the present technology include radioimmunoassay (RIA) (see, e.g., Weintraub, B., Principles of Radioimmunoassays, Seventh Training Course on Radioligand Assay Techniques, The Endocrine Society, (March, 1986)). The agent used to detect the polypeptide-antigen binding complex may be radioactively labeled. The radioactive isotope can be detected by means including, but not limited to, e.g., a gamma counter, a scintillation counter, or autoradiography.

In one embodiment, the invention provides compositions and methods of evaluating the relationship between infection with drug-resistant MTB and cellular immune responses to drug-resistance antigens differentially expressed by MTB during drug resistance. Preferably, detection of a cellular immune response can be accomplished by detecting T cells or T cell responses specific for the antigens of the present invention. Thus, the invention provides an assay for distinguishing between MTB infection that is drug-susceptible and drug-resistant.

In one embodiment, the T cells are in the form of peripheral blood lymphocytes (PBLs). In another embodiment, the T cells are in the form of peripheral blood mononuclear cells (PBMCs). PBLs are mature lymphocytes that are found circulating in the blood, as opposed to being located in organs such as lymph nodes, spleen, thymus, liver or bone marrow.

In a further embodiment, T cells are isolated from body fluids taken from sites of active TB disease, for example bronchoalveolar lavage (BAL) (lung washings) or pleural effusions or cerebrospinal fluid or ascites.

However, it will be understood that any body fluid containing T cells can be used in the methods of the current invention, which are not restricted to T cells from disease sites or blood. The envisaged body fluids include BAL, lung biopsy, sputum (including induced sputum), ascites, pleural fluid, pleural biopsy, lymph node biopsy, joint aspirate, cerebral spinal fluid, soft tissue abscess and any other affected part of the body.

Preferably, the T cell response measured is secretion of one or more cytokines and/or chemokines or expression of one or more markers of T cell activation.

Preferably, the cytokine is IFN gamma. However, it will be apparent to the skilled person that other cytokines, for example TNF-alpha or IL-2, and/or chemokines, for example RANTES, MCP-1 or MIP1-alpha, could be employed alone or in combination in the method of the current invention.

It will be readily apparent to the skilled person that the cytokine or chemokine can be detected by any suitable technique known in the art, for example, ELISPOT or intracellular cytokine staining followed by flow cytometry, or cytokine secretion and capture assay or ELISA or whole-blood ELISA.

In one embodiment, the diagnostic assay according to the invention, is performed on PBMCs obtained from a mammal. The in vitro immune diagnostic assay for drug-resistant TB infection, according to the invention, is performed using a diagnostic kit comprising at least one antigen selected from the group consisting of SEQ ID NOs: 1-98 or any combination thereof. For example, PBMCs are isolated from a mammal and the isolated PBMCs are contacted with one or more of the antigens of the invention in order to determine the reactivity of the PBMCs against the antigen. The number of PBMCs secreting IFN-γ in response to stimulation (via cell culture) with a candidate antigen can be measured to quantify the immune response to the antigens of interest.

PBMCs from patients or subjects requiring testing for MTB exposure or infection may be used to detect or diagnose infection by MTB. Generally, the PBMCs from test subjects or mammals are isolated and then cultured with one or more polypeptides, peptide pools or select peptides from the polypeptide, or nucleic acids (DNA or RNA and their derivatives) encoding the polypeptide, of the invention. After a period of time, cell culture supernatants are collected and cytokine production by the PBMCs is measured. In one embodiment, the amount of cytokine secreted by the PBMCs is compared to a control PBMC sample (e.g., from a patient who has not been exposed to MTB). A greater or lesser amount of cytokine present in the test sample is indicative of exposure to and/or infection by MTB. Similar assays are described in more detail in Dillon, et al., J. of Clinical Microbiol. 38:3285-3290 (2000) and in U.S. Pat. No. 7,387,882. Cytokines which can be measured in the PBMC assay described above include but are not limited to any cytokine which the PBMCs can produce (e.g., IFN-gamma, IL-12, IL-5, and IL-2).

In one embodiment, the assay of the invention measures the magnitude of cellular immunologic responses to proteins upregulated by a drug resistant microbe (e.g., rpoB mutant, rifampicin-resistant MTB). In some instances, blood is drawn from a patient and equal amounts of blood are placed in multiple tubes, whereby one tube represents a negative control (e.g., in the absence of an antigen), another tube represents a positive control (e.g., in the presence of a universal antigen), and another tube coated with one or more of the proteins upregulated by the drug resistant microbe (e.g., rpoB mutant, rifampicin-resistant MTB). After a period of incubation time, the amount of cytokine in each tube is compared, and samples showing strong reactions to proteins upregulated by the drug resistant microbe, in combination with minimal and strong reactions in the negative and positive control tubes, respectively, are deemed to be positive. A positive result indicates infection with a drug resistant microbe. In addition, another tube can be added with antigens used to detect MTB infection, such that the assay provides information on presence or absence of MTB infection and the presence or absence of rifampicin (or other drug) resistance in the infecting strain.

Of course, numerous other assay protocols exist that are suitable for use in the methods of the present invention. The descriptions are intended to be illustrative only and in no way is considered to limit the invention.

In some instances, the antigens of the invention may be used in an assay to detect the reactivity of antibodies present in a biological sample from a mammal. For example, the antigens of the invention are used as immunological probes to assess the pattern of humoral immunity driven by the presence of an infection.

The antigens of the invention may be used in the assays described herein, either alone or in combination with one another. The use of single antigens or any combination of antigens may be suitable for use in the assays described herein provided the assay demonstrates the desired sensitivity and negative predictive values. In certain embodiments, the use of a combination of antigens may result in a sensitivity of approximately, 0.80, 0.85, 0.90, 0.91, 0.92, 0.93, 0.94, 0.95, 0.96, 0.97, 0.98, 0.99, or 1.0 with a negative predictive value of approximately 0.80, 0.85, 0.90, 0.91, 0.92, 0.93, 0.94, 0.95, 0.96, 0.97, 0.98, 0.99, or 1.0. In some embodiments, the values are significant. Comparisons may be performed and significance determined using any of the available statistical analysis tools, alone or in combination with one another, including, for example, student's T-test, chi-square test, Fisher's exact test, analysis of variance (ANOVA), univariate statistical analyses, logistic regression analysis to calculate adjusted odds ratio (OR) and 95% confidence interval (CI). Conrols for any statistically significant demographic variables that might function as confounders (gender, etc) may also be utilized. Differences between values are typically considered significant at p<0.05 or p<0.01, for example. Other statistical analysis tools may also be used.

For instance, the assays may be performed to detect antibodies immunoreactive to only one of the antigens of the invention (e.g., SEQ ID NOs 1-98), without assaying for antibodies reactive to any other antigen. Alternatively, the assay may be performed to detect antibodies immunoreactive to more than one of the antigens of the invention. The assays described herein may also be used with other antigens in combination with one another and/or one or more of the antigens of the invention.

Thus, the invention includes a method of detecting the presence of and/or diagnosing a drug resistant strain in a subject comprising detecting in a biological sample from the subject antibodies against one or more antigens of the invention, wherein the presence of antibodies that bind to the antigen(s) is indicative of infection with a drug resistant strain. In certain embodiments, the method comprises contacting a biological sample derived from a subject with the isolated or purified antigens of the invention for a time and under conditions sufficient for an antigen-antibody complex to form and then detecting the formation of an antigen-antibody complex. Detection of the antigen-antibody complex may be achieved by detecting human immunoglobulin in the complex. In certain embodiments, detection of the antibody may be accomplished by contacting the antigen-antibody complex with a “second” antibody that is immunologically reactive with human immunoglobulin (e.g., anti-human immunoglobulin antibody) for a time and under conditions sufficient for the second antibody to bind to the human immunoglobulin in the complex and then detecting the bound anti-human immunoglobulin. It is preferred that the second antibody is labeled with a detectable marker or reporter molecule.

### Pharmaceutical

The present invention includes methods for stimulating a specific immunogenic TB response in an individual, in order to prevent or reduce the severity of the drug-resistant TB disease, by administering an amount of one or more of the polypeptide molecules or nucleic acid molecules described herein (e.g., in a carrier).

In another aspect, the present invention includes compositions (e.g., vaccine compositions or pharmaceutical compositions) having the polypeptide molecules or nucleic acid molecules described herein, in a physiologically acceptable carrier. The composition can also include or can be co-administered with an immune response enhancer (e.g., an adjuvant, another TB antigen, immunostimulatory cytokine or chemokine).

Additional immunological or nucleic acid assessments can be performed using methods known in the art. Assays, known in the art or those later developed can be used to assess the antigenic TB polypeptides in a sample.

In addition to measuring the presence of antigenic TB polypeptides in a sample, assays exist to determine the efficacy of a TB vaccine (e.g., the extent to which the immune response directed at antigens present during drug resistance is stimulated). These types of assays can be used together to fully assess a person's TB status. For example, an individual who has a TB-specific immunogenic response, but tests negative to the presence of one or more the antigenic TB polypeptides in a sample, is one who is less likely to have drug-resistant MTB infection. However, a person who has a TB-specific immunogenic response and tests positive to the presence of the antigenic TB polypeptides of the present invention is someone who likely has infection with a strain of MTB that is drug resistant.

The efficacy of a TB vaccine at stimulating immune responses to antigens produced during drug resistance can be measured by determining the immunogenic response of the person who received the vaccine. The MTB antigens of the present invention (and immunogenic portions thereof) described herein have the ability to induce an immunogenic response. More specifically, the antigens have the ability to induce proliferation and/or cytokine production (i.e., IFN-gamma, IL-12, IL-5, and IL-2) in T cells, NK cells, B cells and/or macrophages derived from an MTB-infected individual.

The selection of cell type for use in evaluating an immunogenic response to an antigen will, of course, depend on the desired response. For example, IL-12 production is most readily evaluated using preparations containing B-cells and/or macrophages. An MTB-infected individual can be identified by virtue of having mounted a T cell response to antigens of MTB. Such individuals can be identified based on a strongly positive (i.e., greater than about 10 mm diameter induration) intradermal skin test response to MTB proteins using a Purified Protein Derivative (PPD). Individuals who have these responses and who do not have any signs or symptoms of active TB disease are considered latently infected with MTB. T cells, NK cells, B cells and macrophages derived from MTB-infected individuals can be prepared using methods known to those of ordinary skill in the art. For example, a preparation of PBMCs (i.e., peripheral blood mononuclear cells) can be employed without further separation of component cells. PBMCs can generally be prepared, for example, using density centrifugation through Ficoll (Winthrop Laboratories, NY). T cells for use in the assays described herein can also be purified directly from PBMCs. Alternatively, an enriched T cell line reactive against mycobacterial proteins, or T cell clones reactive to individual mycobacterial proteins, can be employed. Such T cell clones can be generated by, for example, culturing PBMCs from MTB-infected individuals with mycobacterial proteins for a period of 2-4 weeks. This allows expansion of only the mycobacterial protein-specific T cells, resulting in a line composed solely of such cells. These cells can then be cloned and tested with individual proteins, using methods known to those of ordinary skill in the art, to more accurately define individual T cell specificity. In general, antigens that test positive in assays for proliferation and/or cytokine production (i.e., IFN-γ and/or IL-12 production) performed using T cells, NK cells, B cells and/or macrophages derived from an MTB-infected individual are considered immunogenic. Such assays can be performed, for example, using the representative procedures described below. Immunogenic portions of such antigens can be identified using similar assays, and can be present within the polypeptides described herein.

The ability of a polypeptide (e.g., an immunogenic antigen, or a portion or other variant thereof) to induce cell proliferation can be evaluated by contacting the cells (e.g., T cells and/or NK cells) with the polypeptide and measuring the proliferation of the cells. In general, the amount of polypeptide that is sufficient for evaluation of about 10⁵ cells ranges from about 10 ng/mL to about 100 μg/mL and preferably is about 10 μg/mL. The incubation of polypeptide with cells may be performed at 37° C. for about six days, although protocols vary. Following incubation with polypeptide, the cells are assayed for a proliferative response, which can be evaluated by methods known to those of ordinary skill in the art, such as exposing cells to a pulse of radiolabeled thymidine and measuring the incorporation of label into cellular DNA. In general, a polypeptide that results in at least a three-fold increase in proliferation above background (i.e., the proliferation observed for cells cultured without polypeptide) is considered to be able to induce proliferation.

The ability of a polypeptide to stimulate the production of IFN-γ, TNF-α, and/or IL-2 in cells can be evaluated by contacting the cells with the polypeptide and measuring the level of IFN-γ, TNF-α, or IL-2 produced by the cells. In general, the amount of polypeptide that is sufficient for the evaluation of about 10⁵ cells ranges from about 10 ng/mL to about 100 μg/mL and preferably is about 10 μg/mL. The polypeptide can, but need not, be immobilized on a solid support, such as a bead or a biodegradable microsphere, such as those described in U.S. Pat. Nos. 4,897,268 and 5,075,109. The incubation of polypeptide with the cells is typically performed at 37° C. for about six days. Following incubation with polypeptide, the cells are assayed for IFN-γ, TNF-α, and/or IL-2 (or other proteins made in response to specific antigen stimulation), which can be evaluated by methods known to those of ordinary skill in the art, such as an enzyme-linked immunosorbent assay (ELISA) or, in the case of IL-12 P70 subunit, a bioassay such as an assay measuring proliferation of T cells. In general, a polypeptide that results in the production of at least 50 pg of IFN-γ per mL of cultured supernatant (e.g., containing 10⁵ T cells per mL) is considered able to stimulate the production of IFN-γ.

In general, immunogenic antigens are those antigens that stimulate proliferation and/or cytokine production (i.e., IFN-γ. and/or IL-12 production) in T cells, NK cells, B cells and/or macrophages derived from at least about 25% of MTB-infected individuals. Among these immunogenic antigens, polypeptides having superior therapeutic properties can be distinguished based on the magnitude of the responses in the above assays and based on the percentage of individuals for which a response is observed. In addition, antigens having superior therapeutic properties will not stimulate proliferation and/or cytokine production in vitro in cells derived from more than about 25% of individuals who are not MTB-infected, thereby eliminating responses that are not specifically due to MTB-responsive cells. Those antigens that induce a response in a high percentage of T cell, NK cell, B cell and/or macrophage preparations from MTB-infected individuals (with a low incidence of responses in cell preparations from other individuals) have superior therapeutic properties.

### Vaccine Compositions, Mode and Manner of Administration

The present invention is partly based on the discovery of differential expression of proteins by drug-resistant MTB isolates. Thus one or more MTB proteins expressed at higher levels (relative to drug susceptible MTB) are available to the immune system and are immunogenic. Hence, the vaccine compositions and methods are designed to augment this immunity, and preferably, to induce it a stage wherein the drug-resistant infection can be prevented or controlled. The vaccine compositions are particularly useful in preventing and/or treating MTB infection in subjects at high risk of, or who already have, such a drug-resistant infection, respectively. The vaccine compositions and methods are also applicable to veterinary uses.

Invention includes a vaccine composition for immunizing a subject against drug resistant MTB infection. Preferred antigens for use in a vaccine composition, alone, in combination, or in linear multimers, include any one or more of the antigens described elsewhere in the context of the diagnostic compositions. For example, one or more of the sequences of SEQ ID NOs: 1-98 can be used for the vaccine.

In one embodiment, the vaccine comprises a fusion protein or peptide multimer which includes a drug-resistant antigen, e.g., a full length protein and/or one or more of the above peptides comprising one or more of SEQ ID NO: 1-98.

One or more of the polypeptides of the present invention can be used in the form of a vaccine or immunological composition designed to prevent or treat drug-resistant MTB infection. In some instances, one or more of the polypeptides of the present invention can be in a form of a conjugate or a fusion protein, which can be manufactured by known methods. In particular, one or more of the sequences of SEQ ID NOs: 1-98 can be fused to one another, or with other proteins, to provide a more effective vaccine composition, and stimulate an improved immunogenic response. Other proteins that can be used to make such a fusion protein include, for example, MTB antigens that simulate the CD4+ T cell or CD1 cellular pathway (which present lipid antigens to T cells) of the immune response. The MTB polypeptides of the present invention were isolated from MHC class 1 molecules, molecules known for presenting antigens to CD8+ T cells. Although it is possible for these polypeptides to be also presented in the CD4+ pathway, fusing a CD4+ T-cell pathway antigen with one of the polypeptides of the present invention can serve to increase effectiveness of the TB vaccine. Fusion proteins can be manufactured according to known methods of recombinant DNA technology. For example, fusion proteins can be expressed from a nucleic acid molecule comprising sequences which code for a biologically active portion of the TB polypeptides or the entire TB polypeptides set forth in SEQ ID NOs: 1-98 or combinations thereof, and its fusion partner, for example another sequence of the present invention, a portion of an immunoglobulin molecule, or another TB antigen from the CD4+ T cell pathway. For example, some embodiments can be produced by the intersection of a nucleic acid encoding immunoglobulin sequences into a suitable expression vector, phage vector, or other commercially available vectors. The resulting construct can be introduced into a suitable host cell for expression. Upon expression, the fusion proteins can be isolated or purified from a cell by means of an affinity matrix. By measurement of the alternations in the functions of transfected cells occurring as a result of expression of recombinant MTB proteins, either the cells themselves or MTB proteins produced from the cells can be utilized in a variety of screening assays.

In certain aspects the inventive compositions comprise fusion proteins or DNA fusion molecules. Each fusion protein comprises a first and a second inventive polypeptide or, alternatively, a polypeptide of the present invention and a known MTB antigen, together with variants of such fusion proteins. The fusion proteins of the present invention can also include a linker peptide between the first and second polypeptides. The DNA fusion molecules of the present invention comprise a first and a second isolated DNA molecule, each isolated DNA molecule encoding either an inventive MTB antigen or a known MTB antigen.

A DNA, or mRNA derived from the DNA sequence by translation using known methods and enzymes, sequence encoding a fusion protein of the present invention is constructed using known recombinant DNA techniques to assemble separate DNA sequences encoding the first and second polypeptides into an appropriate expression vector, as described in detail below. The 3′ end of a DNA sequence encoding the first polypeptide is ligated, with or without a peptide linker, to the 5′ end of a DNA sequence encoding the second polypeptide so that the reading frames of the sequences are in phase to permit mRNA translation of the two DNA sequences into a single fusion protein that retains the biological activity of both the first and the second polypeptides.

A peptide linker sequence can be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences can be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala can also be used in the linker sequence Amino acid sequences which can be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence can be from 1 to about 50 amino acids in length. Peptide sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons require to end translation and transcription termination signals are only present 3′ to the DNA sequence encoding the second polypeptide.

Efficacy of a vaccine including the isolated sequences of the present invention can be determined based on the ability of the antigen to provide at least about a 50% (e.g., about a 60%, about a 70%, about a 80%, about a 90%, or about a 100%) reduction in bacterial numbers and/or at least about a 40% (e.g., about a 50%, about a 60%, about a 70%, about a 80%, about a 90%, or about a 100%) decrease in mortality following experimental infection in a challenge experiment. Suitable experimental animals include but are not limited to mice, guinea pigs, rabbits and primates.

The compositions of the present invention are preferably formulated as either pharmaceutical compositions or as vaccines for in the induction of therapeutic or preventive immunity against drug-resistant TB in a patient. A patient can be afflicted with a disease, or can be free of detectable disease and/or infection. In other words, protective immunity can be induced to prevent, reduce the severity of, or treat drug-resistant TB.

In one embodiment, pharmaceutical compositions of the present invention comprise one or more of the above polypeptides, either present as a mixture or in the form of a fusion protein, and a physiologically acceptable carrier. Similarly, vaccines comprise one or more the above polypeptides and a non-specific immune response enhancer, such as an adjuvant or a liposome (into which the polypeptide is incorporated).

In another embodiment, a pharmaceutical composition and/or vaccine of the present invention can contain one or more of the DNA molecules of the present invention, either present as a mixture or in the form of a DNA fusion molecule, each DNA molecule encoding a polypeptide as described above, such that the polypeptide is generated in situ. In such vaccines, the DNA can be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA can be introduced using a viral expression system (e.g., vaccinia or other pox virus, retrovirus, or adenovirus), which can involve the use of a non-pathogenic (defective), replication competent virus. Techniques for incorporating DNA into such expression systems are well known to those of ordinary skill in the art. The DNA can also be “naked,” as described, for example, in Ulmer et al., Science 259:1745-1749, 1993 and reviewed by Cohen, Science 259:1691-1692, 1993. The uptake of naked DNA can be increased by coating the DNA onto biodegradable beads, which are efficiently transported into the cells.

The antigenic MTB molecules of the present invention can be administered with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation. A carrier (e.g., a pharmaceutically acceptable carrier) is preferred, but not necessary to administer the compound.

The composition can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

The antigenic MTB molecules used in the invention can be administered intravenously, parenterally, intramuscular, subcutaneously, orally, nasally, topically, by inhalation, by implant, by injection, or by suppository. The composition can be administered in a single dose or in more than one dose over a period of time to confer the desired effect.

The actual effective amounts of compound or drug can vary according to the specific composition being utilized, the mode of administration and the age, weight and condition of the patient. For example, as used herein, an effective amount of the drug is an amount which reduces the number of bacteria. Dosages for a particular individual patient can be determined by one of ordinary skill in the art using conventional considerations, (e.g. by means of an appropriate, conventional pharmacological protocol).

For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used.

Pulmonary administration to stimulate immune responses to drug-resistant MTB can be accomplished, for example, using any of various delivery devices known in the art such as an inhaler. See. e.g., S. P. Newman (1984) in Aerosols and the Lung, Clarke and Davis (eds.), Butterworths, London, England, pp. 197-224; PCT Publication No. WO 92/16192; PCT Publication No. WO 91/08760.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

Biodegradable microspheres (e.g., polylactic galactide) can also be employed as carriers for the pharmaceutical compositions of this invention. Suitable biodegradable microspheres are disclosed, for example, in U.S. Pat. Nos. 4,897,268 and 5,075,109.

Any of a variety of adjuvants can be employed in the vaccines of this invention to enhance the immune response to drug resistant antigens. Most adjuvants contain a substance designed to protect the antigen from rapid catabolism, such as aluminum hydroxide or mineral oil, and a nonspecific stimulator of immune responses, such as lipid A, Bortadella pertussis or MTB. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A and quil A.

In one embodiment, it is preferred that the adjuvant induces an immune response comprising Th1 aspects. Suitable adjuvant systems include, for example, a combination of monophosphoryl lipid A, preferably 3-de-O-acylated monophosphoryl lipid A (3D-MLP) together with an aluminum salt. An enhanced system involves the combination of a monophosphoryl lipid A and a saponin derivative, particularly the combination of 3D-MLP and the saponin QS21 as disclosed in WO 94/00153, or a less reactogenic composition where the QS21 is quenched with cholesterol as disclosed in WO 96/33739. Previous experiments have demonstrated a clear synergistic effect of combinations of 3D-MLP and QS21 in the induction of both humoral and Thl type cellular immune responses. A particularly potent adjuvant formation involving QS21, 3D-MLP and tocopherol in an oil-in-water emulsion is described in WO 95/17210 and is a preferred formulation.

The administration of the antigenic MTB polypeptide molecules of the present invention and other compounds can occur simultaneously or sequentially in time. A DNA vaccine and/or pharmaceutical composition as described above can be administered simultaneously with or sequentially to an additional polypeptide of the present invention, a known MTB antigen, an immune enhancer, or other compound known in the art that would be administered with such a vaccine. The compound can be administered before, after or at the same time as the antigenic MTB molecules. Thus, the term “co-administration” is used herein to mean that the antigenic MTB molecules and the additional compound (e.g., immune stimulating compound) will be administered at times to achieve a specific MTB immune response to drug resistance antigens, as described herein. The methods of the present invention are not limited to the sequence in which the compounds are administered, so long as the compound is administered close enough in time to produce the desired effect.

Routes and frequency of administration of the inventive pharmaceutical compositions and vaccines, as well as dosage, may or may not vary from individual to individual and might parallel those currently being used in immunization using the Bacille Calmette-Guerin (BCG) vaccine. In general, the pharmaceutical compositions and vaccines can be administered by injection (e.g., intracutaneous, intramuscular, intravenous or subcutaneous), intranasally (e.g., by aspiration), intralung, or perhaps even orally. Alternate protocols can be appropriate for individual patients. A suitable dose is an amount of polypeptide or DNA that, when administered as described above, is capable of raising an immune response in an immunized patient sufficient to protect the patient from MTB infection for at least 1-2 years. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Using Adaptive Cellular Immune Function to Diagnose Drug Resistance in Active TB and Latent Mycobacterium tuberculosis Infection

Drug resistance in MTB is primarily due to point mutations in genes encoding mycobacterial enzymes (Blanchard et al., 1996, Annu Rev Biochem 65:215-39). For example, resistance to rifampicin, a key first-line anti-tubercular drug, results most commonly from a single nucleotide polymorphism (SNP) in rpoB. While the adaptive immune system is unlikely to specifically detect a mutant protein resulting from a SNP in rpoB, rpoB is an essential gene in MTB and mutated forms of RNA polymerase B plausibly are associated with changes in transcription that result in differential expression of proteins in the organism.

In vitro proteomics experiments were performed to determine if rifampicin resistant mutant progeny strains of drug-susceptible MTB expressed different proteins. In terms of creating a diagnostic assay specifically using memory immune responses to identify patients with rifampicin resistant MTB, experiments were focused on identifying proteins that were detected in the mutant drug resistant isolates but not in the wild-type drug-susceptible parent isolates or were found at higher levels in the resistant isolates.

The experiments disclosed herein were conducted to investigate whether the adaptive immune system can detect specific changes in the proteome of MTB that occur with the emergence of drug resistance. Without wishing to be bound by any particular theory, it is believed that immunologic assays can be used to identify drug resistance in MTB infection. This approach would be particularly useful when specimens containing the pathogen are difficult to be obtained (e.g., sputum scarce TB disease) or cannot be obtained (e.g., in latent MTB infection). Identification of drug-resistant strains of MTB facilitates more effective treatment regimens, which can improve both patient outcome and public health by minimizing the transmission of drug-resistant strains (Brown et al., 2006, J Microbiol Methods 65(2):294-300).

The results presented herein demonstrate that proteomic changes result from development of drug resistance in MTB, and prediction of antigenicity using bioinformatic approaches indicate that these proteins are likely recognized by the human adaptive immune system.

Without wishing to be bound by any particular theory, it is believed that the proof-of-concept results presented herein regarding of whether candidate antigens are differentially-recognized by the adaptive immune system supports the use of the diagnostic approach of the present invention to evaluate other infectious disease scenario including but not limited to patients with HIV, as well as in children and the elderly. Strategies can also be investigated that facilitate triage of culture among patients with newly diagnosed active TB. It is believed that this line of investigation has the potential to increase the accurate identification of patients with drug resistant latent TB and therefore combat the ongoing epidemic of MDR-TB.

### Example 2

### Upregulation of the Phthiocerol Dimycocerosate Biosynthetic Pathway by Rifampicin-Resistant, rpoB-Mutant Mycobacterium tuberculosis

MDR-TB has emerged as a major threat to TB control. Phylogenetically related rifampicin-resistant actinomycetes with mutations mapping to clinically dominant MTB mutations in the rpoB gene show upregulation of gene networks encoding secondary metabolites. Experiments were conducted to compare the expressed proteomes and metabolomes of two fully drug-susceptible clinical strains of MTB (wild type) to that of their respective rifampicin-resistant, rpoB-mutant progeny strains with confirmed rifampicin monoresistance following antitubercular therapy. Each of these strains was also used to infect IFN-γ- and lipopolysaccharide-activated murine J774A.1 macrophages to analyze transcriptional responses in a physiologically-relevant model. Both rpoB mutants showed significant upregulation of the polyketide synthase gene cluster ppsA-E and drrA, which constitute an operon encoding multifunctional enzymes involved in the biosynthesis of phthiocerol dimycocerosate and other lipids in MTB but also of various secondary metabolites in related organisms, including antibiotics such as erythromycin and rifamycins. ppsA (Rv2931), ppsB (Rv2932) and ppsC (Rv2933) were also found to be upregulated greater than 10-fold in the Beijing rpoB mutant strain relative to its wild-type parent strain during infection of activated murine macrophages. In addition, metabolomics identified precursors of phthiocerol dimycocerosate, but not the intact molecule itself, in greater abundance in both rpoB mutant isolates. These data suggest that rpoB mutation in MTB triggers compensatory transcriptional changes in genes, such as secondary metabolism genes, analogous to those observed in related Actinobacteria. These findings assist in developing novel methods to diagnose and treat drug resistant MTB infections.

The materials and methods employed in these experiments are now described.

### Materials and Methods

Mtb Strains

All MTB strains were obtained from patients being treated for pulmonary TB. Isolates were sent to National Jewish Hospital, Denver, Colo., for confirmation of drug susceptibility data that was initially obtained by the clinical labs where the patients were being treated. An MTB strain susceptible to all drugs tested (rifampicin, isoniazid, pyrazinamide, and ethambutol) was obtained from a patient living in Costa Rica prior to initiating antitubercular therapy. Another isolate was later obtained from the same patient after several weeks on treatment and was confirmed to be resistant to rifampicin by the Agar proportion test (MIC>1 mg/L) but susceptible to the other 3 first line agents. These isolates were a parent-mutant pair by spoligotyping and insertion sequence (IS)6110 restriction fragment length polymorphism (RFLP) pattern. Spoligotyping analysis determined these isolates belong to the Beijing family. Analysis of genomic DNA from these strains revealed an H445D mutation in the rpoB gene of the rifampicin-resistant isolate. Both isolates also had an A463G mutation in katG but were both confirmed susceptible to isoniazid (MIC<0.2 mg/L) and these mutations were considered clinically silent (Haas et al., 1997, Antimicrob Agents Chemother 41:1601-3; Rouse et al., 1995, Antimicrob Agents Chemother 39:2472-7) Similarly, an Mtb strain susceptible to all drugs tested (rifampicin, isoniazid, pyrazinamide, and ethambutol) was obtained from a patient in San Francisco prior to initiating standard-first line anti-tubercular therapy. After several weeks on therapy, another isolate was obtained from the same patient and was confirmed to be resistant to rifampicin by Agar proportion test but susceptible to the other 3 first-line agents. These isolates were also confirmed as paired by spoligotyping and (IS)6110 RFLP, and were determined to belong to the Haarlem family. Analysis of genomic DNA from these two Haarlem isolates revealed that the rifampicin resistant isolate had an S450L mutation in the rpoB gene. Both isolates also had a mutation at position 103 of the rpoB gene, and the wild-type strain had an additional mutation at position 108 of oryR. The rpoB mutations present in the resistant isolates are two of the most common rifampicin-resistance mutations encountered clinically in adults with TB (Telenti et al., 1993, Lancet 341:647-50).

For laboratory-based rpoB mutant generation used in confirmatory experiments, an exponentially-growing culture of Johns Hopkins University wild-type lab reference strain MTB CDC1551 (Ahmad et al., 2009, J Infect Dis 200:1136-43) was inoculated onto 7H10 agar containing 1 μg/ml of rifampicin and incubated at 37° C. for 21 days. Individual colonies were inoculated separately in Middlebrook 7H9 broth containing 1 μg/ml of rifampicin and grown at 37° C. to OD600˜1.0. Genomic DNA was purified from each culture, and the rpoB gene was amplified using primers 5′-AATATCTGGTCCGCTTGCAC-3′ (SEQ ID NO: 99) and 5′-ACACGATCTCGTCGCTAACC-3′ (SEQ ID NO: 100), and sequenced. Based on the sequencing results, a mutant containing a substitution of G for C at position 1351 bp in the rpoB gene (yielding the RpoB S450L mutation) was selected for further study (Table 1).

TABLE 1
primers used for cDNA synthesis

| Gene name | SEQ ID No | Primer sequence |
|---|---|---|
| rpoB | 99 | AATATCTGGTCCGCTTGCAC |
| rpoB | 100 | ACACGATCTCGTCGCTAACC |

Culture Conditions

For proteomics studies, a glycerol stock of each strain was plated in Middlebrook 7H11 (Difco), supplemented with Oleic Acid Albumin Dextrose (OADC). After incubation for 2 weeks at 37° C., colonies were inoculated in 100 ml of Middlebrook 7H9 (Difco) supplemented with OADC and 0.05% Tween. The mycobacteria were further cultured at 37° C. in agitation for two weeks, and then were washed twice with sterile PBS (Invitrogen) and inoculated in 1 L of GAS media (Glycerol, Alanine, Salts) (Takayama et al., 1972, Antimicrob Agents Chemother 2:29-35), a traditional Mycobacterium spp. medium used to process cells and spent filtrate for downstream proteomics applications (Barnes et al., 1989, J Immunol 143:2656-62), (Mehaffy et al., 2010, Proteomics 10:1966-84), (Sartain et al., 2006, Mol Cell Proteomics 5:2102-13). Cultures were then incubated at 37° C. in agitation for 4 weeks. All cultures were prepared in triplicate for proteomics analyses. For metabolomics studies, a glycerol stock of each strain was plated in Middlebrook 7H11 (Difco), supplemented with OADC. Cultures were incubated at 37° C. for 4 weeks. Five replicates per strain were prepared for metabolomics studies.

Culture Filtrate Proteins (CFP)

Proteomics data were obtained within specific cellular fractions, consisting of culture filtrate (CFP), membrane-associated (MEM), cell wall (CW), and cytoplasm (CYT) proteins. CFP was purified as described elsewhere (Mehaffy et al., 2010, Proteomics 10:1966-84). Briefly, each culture was filtered using a 0.2 um zap-cap filter, Whatman (GE Healthcare, Piscataway, N.J.). Secreted proteins were recovered from the culture filtrate as follows: 1 L culture filtrates were concentrated to approximately 25 ml of volume using a 10 KDa MWCO membrane (Millipore). Then, the filtrate was further concentrated to approximately 300 μl using an Amicon Ultracell-15 with a 10 KDa MWCO by centrifugation at 3000 rpm at 4° C. Purified proteins were subjected to buffer exchange using 15 ml of 10 mM ammonium bicarbonate three times in the same filter unit. Protein quantification was performed by the BCA assay (Pierce).

Subcellular Fractions

Cells were pelleted by centrifugation at 3000 rpm and washed twice with 10 ml of sterile PBS (Invitrogen). Harvested cells were inactivated with 2.4 Mrad of cesium γ-irradiation for 24 hr and death was confirmed by Alamar Blue assay (Invitrogen). Cells were resuspended in 10 ml of breaking buffer (1 mM EDTA-PBS supplemented with one tablet of protease inhibitor (Roche Diagnostics) per 50 ml of buffer, 60 μl of DNAse (1 mg/ml) and 60 μl of RNAse (1 mg/ml)) and broken by sonication on 50% duty cycle (12 times, 60 sec with intervals of 90 sec on ice). After sonication, breaking buffer was added to a final volume of 40 ml and unbroken cells were removed by centrifugation at 3000 rpm for 5 min, 4° C. Supernatant was further centrifuged for one hour at 27,000×g 4° C. to separate the cell wall pellet from cytosol and membrane fractions (supernatant). The cell wall pellet was resuspended in 10 mM ammonium bicarbonate and stored at −80° C. Finally, membrane proteins were harvested by ultracentrifugation at 100,000×g for 8 hours (2×4 h). The cytosol fraction (supernatant) was concentrated by centrifugation using Amicon Ultra-15 centrifugal filter units with a 10 KDa MWCO. Buffer exchange using 15 ml of 10 mM ammonium bicarbonate was performed three times using the same filter unit. Membrane fraction was resuspended in 10 mM ammonium bicarbonate and stored at −20° C. All subcellular fractions were quantified using the BCA assay (Pierce).

In-Solution Protein Digestion

Fifty micrograms of CFP, MEM, and CYT fractions were precipitated with acetone following standard protocols. Protein pellets were resuspended in 15 μl of 8 M urea followed by addition of 20 μl of 0.2% ProteaseMAX Surfactant (Promega) in 50 mM ammonium bicarbonate. After vortexing for 5 min, 58.5 μl of 50 mM ammonium bicarbonate were added to each sample. Proteins were reduced after incubation at 50° C. for 20 mM with 5 mM DTT. Alkylation was performed with 15 mM iodoacetamide followed by incubation at RT for 15 min in the dark. Samples were digested for 3 hr at 37° C. with 1 μl of 1% ProteaseMAX™ Surfactant (Promega) in 50 mM ammonium bicarbonate and 1.8 μg of trypsin. Reactions were stopped with 0.5% trifloroacetic acid (TFA). All samples were desalted with Pierce PepClean C18 spin columns per manufacture's protocol and eluted with 70% ACN, 0.1% formic acid (FA). Samples were dried in a speedvac and resuspended in 5% ACN, 0.1% FA prior to mass spectrometry analysis. Biological replicates within each cellular fraction were randomized and injected in triplicate. Mass spectrometry analysis was performed at the Proteomics and Metabolomics Facility, Colorado State University, CO. CW proteins were subjected to a dilapidation protocol before digestion as follows: 10 μg of lysosyme was added to 1 mg of CWP and incubated for 30 min at 37° C. Samples were dried in a speedvac and resuspended in 7 ml of chloroform: methanol (2:1 v/v) followed by incubation at room temperature for 1 hr and centrifugation at 3000 rpm for 30 min at 4° C. Pelleted proteins were dried and resuspended in 7 ml of chloroform:methanol:water (10:10:3) followed by incubation in agitation at room temperature for 1 hr and centrifugation at 3000 rpm for 2 h at 4° C. Finally, pelleted proteins were precipitated with 2 ml of cold acetone and incubated overnight at −20° C. After centrifugation at 13000 rpm for 30 min, the protein pellet was resuspended in 10 min ammonium bicarbonate and quantification was performed using the BCA assay (Pierce). Fifty μg of sample were subjected to trypsin digestion, as described elsewhere herein.

Liquid Chromatography/Tandem Mass Spectrometry (LC/MS/MS) for Proteomics Studies

All samples were randomized and analyzed in triplicate by liquid chromatography coupled with tandem mass spectrometry. Briefly, peptides were purified and concentrated using an on-line enrichment column (Agilent Zorbax C18, 5 μm, 5×0.3 mm) Subsequent chromatographic separation was performed on a reverse phase nanospray column (Agilent 1100 nanoHPLC, Zorbax C18, 5 μm, 75 μm ID×150 mm column) using a 60 minute linear gradient from 25%-55% buffer B (90% ACN, 0.1% formic acid) at a flow rate of 300 nanoliters/min Peptides are eluted directly into the mass spectrometer (Thermo Scientific Linear Trap Quadrupole (LTQ) linear ion trap) and spectra are collected over a m/z range of 200-2000 Da using a dynamic exclusion limit of 2 MS/MS spectra of a given peptide mass for 30 s (exclusion duration of 90 s). Compound lists of the resulting spectra were generated using Bioworks 3.2 software (Thermo Scientific) with an intensity threshold of 5,000 and 1 scan/group. Spectra were subjected to interrogation against the MTB genome (including reverse strand) (Genbank accession #AL123456, R9, 7,982 entries) using SORCERER (Sage-N Research, version 2.0) and SEQUEST (Thermo Fisher Scientific, Release 27, rev 12). All searches were performed assuming trypsin digestion, 4 missed cleavages, fragment ion mass tolerance of 1.00 Da, and a parent ion tolerance of 1.5 Da. Carbamidomethyl (C) and Oxidation (M) were specified as variable modifications. Analyses (per subcellular fraction) were compiled in Scaffold (version 3.00.04, Proteome Software Inc, Portland, Oreg.) in order to validate MS/MS based peptide and protein identification. Search results for triplicate injections of each biological replicate were summed together upon compilation within the Scaffold software. In addition, the X! Tandem database search algorithm (version 2007.01.01.2, The Global Proteome Machine, www.thegpm.org) was used to verify and complement the peptide identifications from Sequest. Peptide identifications were filtered by database search engine thresholds, such that Sequest identifications required deltaCn scores greater than 0.10 and XCorr scores greater than 1.0, 2.0, 3.0 and 3.0 for singly, doubly, triply, and quadruply charged peptides, respectively. X! Tandem identifications required −Log(Expect Scores) scores greater than 2.0. Protein identifications were accepted if they could be established at greater than 99.0% probability (Protein Prophet algorithm) and contained at least 1 unique peptide (Keller et al., 2002, Anal Chem 74:5383-92). False discovery rates (FDR) were calculated automatically by Scaffold (version 3.0) using the decoy database and reverse hits and the empirical method previously reported by Kall et al (Kall et al., 2008, J Proteome Res 7:29-34). Analysis of each dataset using these parameters resulted in a false discovery rate (FDR) of 0.2% at the protein level and less than 5.3% at the peptide level. After statistical analysis, differential proteins that were identified by only one unique peptide were subjected to manual spectral validation using the following criteria: (1) minimum of 80% coverage of theoretical y or b ions (at least 5 in consecutive order), (2) absence of prominent unassigned peaks greater than 10% of the maximum intensity, and (3) indicative residue-specific fragmentation, such as intense ions proximal (N-terminal) to proline and immediately distal (C-terminal) to aspartate and glutamate. Data, exported as Mascot generic files and containing all MS/MS spectra, are available on the Proteomics IDEntifications database for review (Vizcaino et al., 2010, Nucleic Acids Res 38:D736-42).

Proteomic Data Analysis

Each subcellular fraction dataset was subjected to spectral count analysis using Scaffold (version 3.00.04, Proteome Software Inc, Portland, Oreg.). Technical replicates for each biological replicate were summed within the Scaffold software and each subcellular fraction data set was analyzed independently. Prior to all analyses of differential protein detection, spectral counts were normalized against the total signal (per biological replicate) as described previously (Carvalho et al., 2008, Genet Mol Res 7:342-56). Using spectral counts, differential protein detection was assessed in multiple ways. First, after normalization, data was log transformed and a linear model analysis of variance (ANOVA) comparing spectral counts for each protein within subcellular fractions for each isogenic pair was applied to identify proteins that were differentially abundant between susceptible and resistant isolates (p-value <0.05). Comparison of spectral counts was performed using DanteR (version 1.0.1.1. Pacific Northwest National Laboratory, http://omics.pnl.gov). Reproducibility of biological replicates was assessed by visual interpretation of each dataset using box plots and 3D scatter plots generated on DanteR. In addition, to ascertain global differences in protein detection between rpoB-mutant and wild-type isolates, the mean spectral counts in the cell-associated fractions (CW, MEM, CYT) were added to produce a summary spectral count for each protein. CFP was not included in the summary spectral count given our interest in measuring differences in overall protein abundance and not secretion. This summary cell-associated spectral count was then used to calculate the fold change in detection for each protein by dividing the mean spectral count in an rpoB mutant by the mean spectral count in the paired wild-type parent isolate. The summary cell-associated spectral counts were also used to calculate the spectral index (SpI), which is a robust statistical measure of differential protein abundance useful in shotgun proteomics experiments (Fu et al., 2008, J Proteome Res 7:845-54). The SpI, which ranges from −1 to +1, was then used to rank-order the proteins by degree of increased detection in mutants. SpI values of or near 0 indicate that the relative peptide abundance is approximately the same in the rifampicin-resistant and rifampicin susceptible cultures, whereas positive or negative values indicate enrichment in the rifampicin-resistant or rifampicin-susceptible cultures, respectively (Fu et al., 2008, J Proteome Res 7:845-54). With this ranking, Gene Set Enrichment Analysis (GSEA) at the functional class level was then applied to identify significantly upregulated gene sets in the resistant strains (Subramanian et al., 2005, Proc Natl Acad Sci USA 102:15545-50). Because rpoB mutation appears to significantly upregulate genes specifically involved in biosynthesis of natural product/secondary metabolites in other organisms (Carata et al., 2009, Microb Cell Fact 8:18; Hosaka et al., 2009, Nat Biotechnol 27:462-4; Hu et al., 2002, J Bacteriol 184:3984-91; Inaoka et al., 2004, J Biol Chem 279:3885-92), an automated approach was also used to search the Mtb genome for gene clusters potentially associated with natural product biosynthesis (NP.searcher, http://dna.sherman.lsi.umich.edu/) (Li et al., 2009, BMC Bioinformatics 10:185) and compared the expression of these gene clusters by mutants and wild-type MTB using GSEA.

Macrophage Studies

The murine macrophage J774A.1 cell line was cultured in Roswell Park Memorial Institute (RPMI) media supplemented with 0.2 mM L-glutamine (Invitrogen) and 10% heat-inactivated fetal bovine serum (FBS) (Sigma) in a humidified 37° C., 5% CO₂ incubator. After growth to a confluent monolayer, the cells were harvested using a sterile rubber scraper and a single cell suspension was prepared and cultured in a 75 cm² cell culture flask (Costar). A total of 103 cells were activated by addition of 50 ng/ml IFN-γ (Roche) overnight and 200 ng/ml lipopolysaccharide (LPS) (from Escherichia coli 026:B6, <5% protein (Lowry), Sigma) for 3 hours prior to infection. After removal of the media, the cells were incubated for 3 hours with either wild-type MTB or the rpoB mutant at a multiplicity of infection (MOI) of 1:1. The macrophages were then treated with 200 ng/ml streptomycin and washed three times with RPMI and cultured in media as described elsewhere herein. For bacterial enumeration for comparison of growth rates of mutant and WT-isolates, the cells were lysed with Triton X-100 at predetermined time points and the lysate was serially diluted and plated on 7H10 agar and incubated at 37° C. for 28 days, as previously described (Thayil et al., 2011, PLoS One 6:e28076). For mycobacterial gene expression studies, intracellular bacteria were recovered at 72 hr after infection by centrifugation at 3200 rpm for 5 min, and the bacterial pellet was resuspended in Trizol reagent (Invitrogen).

Mtb Gene Expression Studies

Macrophage and mycobacterial membranes were disrupted using 0.1 mm zirconia/silica beads in a bead beater, and RNA was recovered by centrifugation, chloroform extraction, and isopropyl alcohol precipitation, as previously described (Karakousis et al., 2008, J Antimicrob Chemother 61:323-31; Karakousis et al., 2004, J Exp Med 200:647-57). Prior to reverse transcription, control and mutant RNA (10 ng) were treated with RNase-free DNase (Invitrogen) and subjected to 36 cycles of PCR to ensure that all DNA had been removed, as assessed by ethidium bromide-stained agarose gel analysis. Fluorescently-labeled cDNA was generated using gene-specific primers (Table 2) and Superscript III (Invitrogen), as previously described (Thayil et al., 2011, PLoS One 6:e28076). cDNA corresponding to each transcript was subjected to 40 cycles of PCR for quantification using the primers listed in Table 3 and an iCycler 5.0 (Bio-RAD). The cycle threshold value (CT) obtained for each gene of interest (GOI) was normalized with that of sigA, a housekeeping gene (HKG) with constant expression under different experimental conditions (Manganelli et al., 1999, Mol Microbiol 31:715-24), in order to obtain the normalized CT (nCT=(GOI CT)−(HKG CT)). The change in normalized CT (ΔnCT) for each gene was calculated using the following formula: (ΔnCT=C(nCT)−S(nCT)), where C represents the wild-type (control) strain and S represents the rpoB mutant strain. Fold regulation of individual genes was calculated using the following formula: 2−ΔnCT. The data are representative of three biological replicates, and the experiments were repeated twice.

TABLE 2
Gene-specific primers used for cDNA synthesis

| Gene name | Primer name | Primer sequence | SEQ ID |
|---|---|---|---|
| ppsA | d195 | ATCTTCGGCCACATAACCTG | 101 |
| ppsB | d196 | GAGAGGCTGTCTACCCGAGA | 102 |
| ppsC | d197 | GAGAAGGTCAGCCACCAGTC | 103 |
| Rv0799c | d191 | ATGGTCGAGAAAGTCGATGG | 104 |
| Rv0496 | RT-Rv0496-R | TTTCCACCGCTTCTATCGAC | 105 |
| sigA | sigA-R | GGTGATGTCCATCTCTTTGG | 106 |

TABLE 3
Primer sets used for qRT-PCR in macrophage studies

| Gene | Primer | Primer | SEQ |
|---|---|---|---|
| name | name | sequence | ID |
| ppsA | dl93 | TCCGGATCTGTACCGAAATC | 107 |
| | dl94 | ATTCGCCTAAGTCGTCGTTG | 108 |
| ppsB | dl77 | GACTACCCGCAGGTGACAGT | 109 |
| | dl78 | GTGGAGATGATTCCGATGGT | 110 |
| ppsC | dl79 | CCTCGACCTGAATCTCAAGC | 111 |
| | dl80 | ATCTTGCCGGTGTGTTTACC | 112 |
| Rv0799c | dl71 | GACATCGAACTCGACGACAA | 113 |
| | dl72 | CGGTGGAAAAGTCAAGCACT | 114 |
| Rv0496 | RT-Rv0496-F | AGAGGACCCTAACGGCAAAT | 115 |
| | RT-Rv0496-R | TTTCCACCGCTTCTATCGAC | 116 |
| sigA | sigA-F | CTACGCTACGTGGTGGATTC | 117 |
| | sigA-R | GGTGATGTCCATCTCTTTGG | 118 |

Extraction of MTB Metabolites

Mycobacteria were scraped at 4 weeks of culture and transferred to a glass tube. 10 ml of chloroform was added to each sample and then incubated at room temperature in agitation for 4 hours. Bacteria were pelleted at 3000 rpm for 30 min at 4° C. and the supernatant corresponding to the chloroform fraction was dried in a nitrogen bath and stored at −20° C. Pelleted bacteria were dried under nitrogen and then resuspended in 15 ml of methanol. Samples were incubated at 37° C. overnight in agitation and then centrifuged at 3000 rpm for one hour at 4° C. Supernatant corresponding to the methanol fraction was dried under nitrogen and then stored at −20° C. Prior to mass spectrometry analysis, chloroform and methanol fractions were resuspended in 1 ml of the corresponding solvent.

LC/MS for Metabolomics

Methanol fractions: One μL, of the sample was injected into a Waters Acquity UPLC system equipped with a Waters Acquity UPLC T3 column (1.8 μM, 1.0×100 mm) Separation was achieved using a gradient from solvent A (95% water, 5% methanol, 0.1% FA) to solvent B (95% methanol, 5% water, 0.1% FA) as follows: 100% A held for 1 min; 100% B in 16 min; 100% B held for 3 min; 100% A in 0.1 min; re-equilibrate for 5.9 min (total run time of 26 min/sample).

Chloroform Fractions:

One μL of the sample was injected on a Waters Acquity Ultra Performance Liquid Chromatography (UPLC) system equipped with a Waters Acquity UPLC C8 column (1.8 μM, 1.0×100 mm) Separation was achieved using a gradient from solvent A (89% water, 5% ACN, 5% isopropanol, 1% 500 mM ammonium acetate) to solvent B (49.5% ACN, 49.5% isopropanol, 1% 500 mM ammonium formate) as follows: 100% A held for 0.1 min; 40% B in 0.9 min; 100% B in 10 min; 100% B held for 3 min; 100% A in 0.1 min; re-equilibrate for 5.9 minutes (total run time of 20 min/sample). MS/MS spectra of precursor ions 797.7 and 823.2 were collected using a Waters UPLC coupled to a Waters Xevo G2 Q-TOF MS. The LC conditions were the same as listed above, and the MS/MS collision energy was ramped from 15-30 volts to induce fragmentation.

For both fractions, flow rate was maintained at 140 μL/min for the duration of the run, the column was held at 50° C., and samples were held at 5° C. Column eluate was infused into a Waters Q-Tof Micro MS fitted with an electrospray source. Data was collected in positive ion mode, scanning from 50-1200 (methanol fraction) or 50-1800 (Chloroform fraction) at a rate of 0.9 seconds per scan with 0.1 second interscan delay. Calibration was performed prior to sample analysis via infusion of sodium formate solution, with mass accuracy within 5 ppm. During analysis, the capillary voltage was held at 2200V, the source temp at 130° C., the desolvation temperature at 300° C., the desolvation gas flow rate of 400 L/hr N2(g), and the quadrupole was held at collision energy of 7 volts. MS/MS spectra of precursor ions 797.7 and 823.2 were collected using a Waters UPLC coupled to a Waters Xevo G2 Q-TOF MS. The LC conditions were the same as listed elsewhere herein, and the MS/MS collision energy was ramped from 15-30 volts to induce fragmentation.

Metabolomics Data Analysis

Raw data files were converted to .cdf format, and feature detection and alignment was performed using XCMS in the program R. Raw peak areas were normalized to total ion signal in R, and the normalized dataset was subjected to PCA in R using the pcaMethods package. Consistent with our focus on gene upregulation as a compensatory response to rpoB mutation, statistical analysis to determine peaks/features that were significantly more abundant in resistant strains was performed in DanteR as described for the proteomics section. Differential peaks (ANOVA p value <0.05) were grouped by retention time and then manually validated using MassLynx (Waters Corporation). Peaks with the same retention time and peak shape were considered to belong to the same compound. Retention time and the inferred molecular weight of each differential peak were used to interrogate public databases including METLIN (http://metlin.scripps.edu) (Smith et al., 2005, Ther Drug Monit 27:747-51), KEGG (http://www.genome.jp/kegg/), Lipid Maps (http://www.lipidmaps.org), and MassBank (http://www.massbanks.jp) (Herrera et al., 2003, Int J Antimicrob Agents 21:403-8). Given the proteomics and metabolomics results, a PDIM standard was subjected to tandem mass spectrometry analysis to gain insight into the structure of the m/z 797.74 ion.

Thin Layer Chromatography

200 ug of lipid fractions were analyzed by Thin Layer Chromatography along with PDIM standard (bug) using 98:2 petroleum ether:ethyl acetate (v/v) three times. Lipids were visualized with CuSO4 charring.

The results of the experiments are now described.

Proteomic Analysis of rpoB-Mutant Strains of MTB

To compare the proteomes of Beijing and Haarlem strains of rpoB-mutant, rifampicin resistant MTB to those of their wild-type progenitor isolates, proteins from four cellular fractions were extracted from late-log phase broth cultures. By separating the sample into less complex parts, such as subcellular fractions, protein identification using shotgun proteomic analysis is greatly increased (Dreger, 2003, Mass Spectrom Rev 22:27-56; Huber, et al, 2003, Circ Res 92:962-8). In addition, because of the insoluble nature of cell wall proteins, this fraction is subjected to additional preparation prior to mass spectrometry analysis to increase solubility and the number of identified proteins. After digestion into tryptic peptides, each fraction was analyzed independently in triplicate by LC-MS/MS. The spectra were searched against the MTB genome, and the total number of unique peptides identified in each cellular fraction of each biological replicate by strain type is shown in Table 4. A total of 452, 1,075, 807, and 735 proteins were confidently identified in the CFP, CW, MEM, and CYT, respectively, of the Haarlem isolates, and 455, 1,072, 760, and 704 proteins were confidently identified in the CFP, CW, MEM, and CYT fractions, respectively, of the Beijing isolates.

TABLE 4
Number of unique peptides identified for each biological
replicate within cellular fractions by MTB strain type.
H = Haarlem; R = rifampicin resistant; B = Beijing; S = rifamipcin susceptible

| | Haarlem RIF- | | | Haarlem RIF- | | | Beijing RIF- | | | Beijing RIF- | | |
|---|---|---|---|---|---|---|---|---|---|---|---|---|
| | resistant | | | susceptible | | | resistant | | | susceptible | | |
| | M. tuberculosis | | | M. tuberculosis | | | M. tuberculosis | | | M. tuberculosis | | |
| Biological replicate | 1 | 2 | 3 | 1 | 2 | 3 | 1 | 2 | 3 | 1 | 2 | 3 |
| Cytosol | 2021 | 2120 | 2204 | 2033 | 1995 | 2180 | 2167 | 2031 | 1951 | 2052 | 1985 | 2165 |
| Membrane | 1572 | 1650 | 1359 | 1354 | 1409 | 1115 | 1560 | 1301 | 1454 | 1050 | 1353 | 1339 |
| Cell wall | 1944 | 1888 | 1921 | 2104 | 1343 | 1801 | 1943 | 1995 | 1921 | 1943 | 1850 | 1403 |
| Culture | 991 | 1082 | 973 | 1057 | 1053 | 963 | 1214 | 1072 | 1084 | 739 | 921 | 903 |
| filtrate | | | | | | | | | | | | |

Spectral counts for each biological replicate were normalized against the total signal as described previously (Carvalho et al., 2008, Genet Mol Res 7:342-56). After normalization and log transformation of the data, a linear model ANOVA comparing spectral counts for each protein within subcellular fractions for each isogenic pair was applied to identify proteins that were differentially abundant between susceptible and resistant isolates (p-value <0.05). Thirty-two, 44, 21, and 27 proteins were identified with significantly higher spectral counts (p<0.05) relative to the wild-type isolate in the CFP, CW, MEM, and CYT, respectively, of the Haarlem rpoB mutant. Similarly, there were 45, 44, 43, and 31 proteins detected with significantly higher spectral counts in the CFP, CW, MEM, and CYT fractions, respectively, of the Beijing rpoB mutant isolate relative to its wild-type progenitor isolate. A comparison of both rpoB mutants against their respective wild-type, rifampicin-susceptible parent revealed 10 proteins with statistically significantly (p<0.05) higher normalized spectral counts (Table 5). All of the products identified at significantly higher spectral counts in the cell wall fraction of both rpoB mutant, rifampicin-resistant strains represented proteins that are transcriptionally coupled on a 50-kb region involved in the biosynthesis of PDIM in MTB (Camacho et al., 2001, J Biol Chem 276:19845-54; Trivedi et al., 2005, Mol Cell 17:631-43), including two type-I polyketide synthase genes (Rv2933/ppsC and Rv2935/ppsE) and a probable daunorubicin imycoserosate (DIM) transport protein (Rv2936/drrA). Other proteins identified in other cellular fractions besides the cell wall included a succinate semialdehyde dehydrogenase (Rv0234c), a putative integration host factor (Rv1388/mihF), a probable acyl-coA dehydrogenase (Rv3562/fadE31) involved in lipid degradation and a polynucleotide phosphorylase/polyadenylase (Rv2783c/gpsI) involved in mRNA degradation. The final three products identified (Rv1056, Rv3038c, and Rv3661) are conserved hypotheticals of unknown function (Table 5). Spectral counts of recA and dnaE2 were specifically examined using qRT-PCR (Bergval et al., 2007, FEMS Microbiol Lett 275:338-43) but increased spectral counts for the corresponding proteins were not observed. Table 6 shows the number of proteins detected in the Haarlem, Beijing, and both rpoB mutants at summary (combined CW, MEM, and CYT) spectral counts at least 2-fold or higher compared to the wild-type isolates as well as the number of proteins detected in the mutant isolates alone (where fold change is set arbitrarily to 100). There were 25 proteins detected in neither wild-type isolate and in both rpoB mutants (Table 6). Five of these proteins were detected in at least 2 replicates in both rpoB mutants, as shown in Table 7. Table 7 lists the Rv numbers, spectral counts, fold changes, replicate raw data, and p values for the comparison of the two strains for the 90 cell-asociated proteins detected >2 fold higher in both rpoB mutants isolates compared to wild-type, including proteins only detected in the mutant strains. Of the 10 proteins identified at statistically significantly higher counts in specific cellular fractions of both rpoB mutants (Table 5), 3 also had summary cell-associated (e.g., combined spectral counts of all fractions but CFP) spectral counts at least 2-fold higher in both mutant versus wild-type isolates and of these, 2 of 3 involved the PDIM biosynthetic locus (ppsC and drrA). The fold increased detection in the rpoB mutant Haarlem and Beijing isolates for ppsC and drrA were 3.6 and 10.2 and 100 (detected in mutant but not wild-type) and 4.7, respectively. The fold change values for ppsE were 3.07 and 1.97 for Haarlem and Beijing strains, respectively.

TABLE 5
Mean spectral counts and functional classes of proteins detected at statistically
significantly higher spectral counts in specific cellular fractions of both rpoB mutant
versus wild-type parent strains of MTB. Spectral counts are the mean of 3 biological
replicates for each isolate. WT = wild-type

| Protein name | | | | Haarlem | Haarlem | | Beijing | Beijing | |
|---|---|---|---|---|---|---|---|---|---|
| (gene) | Rv number | Fraction | Functional class | rpoB | WT | P value | rpoB | WT | P value |
| Succinic | Rv0234c | CYT | Intermediary | 29 | 21 | 0.003 | 24 | 15 | 0.002 |
| semialdehyde | | | metabolism and | | | | | | |
| dehydrogenase | | | respiration | | | | | | |
| (gabD1) | | | | | | | | | |
| Hypothetical | Rv1056 | CFP | Conserved | 32 | 11 | 0.029 | 41 | 17 | 0.037 |
| protein Rv1056 | | | hypothetical | | | | | | |
| Putative | Rv1388 | CFP | Information | 5.35* | 0 | 0.022 | 4.61 | 0 | 0.009 |
| integration host | | | pathways | | | | | | |
| factor MIHF | | | | | | | | | |
| (mihF)* | | | | | | | | | |
| Probable | Rv3562 | CFP | Lipid | 2.34 | 0 | 0.004 | 2.05 | 0 | 0.002 |
| acyl-coA | | | metabolism | | | | | | |
| dehydrogenase | | | | | | | | | |
| FADE31 | | | | | | | | | |
| (fadE31) | | | | | | | | | |
| Polynucleotide | Rv2783c | MEM | Information | 74.78 | 44.32 | 0.038 | 95.96 | 54.98 | 0.003 |
| phosphorylase/ | | | pathways | | | | | | |
| polyadenylase | | | | | | | | | |
| (gpsI) | | | | | | | | | |
| Hypothetical | Rv3038c | MEM | Conserved | 10.87 | 1.37 | 0.014 | 3.89 | 0.66 | 0.045 |
| protein Rv3038c | | | hypothetical | | | | | | |
| Hypothetical | Rv3661 | MEM | Conserved | 9.43 | 0.73 | 0.012 | 7.29 | 2.51* | 0.007 |
| protein | | | hypothetical | | | | | | |
| Rv3661* | | | | | | | | | |
| Phenolpthiocerol | Rv2933 | CW | Lipid | 16.32 | 4.98 | 0.001 | 41.74 | 3.66 | 0.029 |
| synthesis type-I | | | metabolism | | | | | | |
| polyketide | | | | | | | | | |
| synthase (ppsC) | | | | | | | | | |
| Phenolpthiocerol | Rv2935 | CW | Lipid | 41.69 | 19.75 | 0.049 | 65.64 | 32.15 | 0.041 |
| synthesis type-I | | | metabolism | | | | | | |
| polyketide | | | | | | | | | |
| synthase (ppsE) | | | | | | | | | |
| Probable | Rv2936 | CW | Cell wall | 3.83 | 0 | 0.012 | 6.55 | 1.41 | 0.034 |
| daunorubicin- | | | and cell | | | | | | |
| DIM-transport | | | processes | | | | | | |
| ATP-binding | | | | | | | | | |
| protein ABC | | | | | | | | | |
| transporter (drrA) | | | | | | | | | |

Numbers correspond to normalized spectral counts
*Protein identified by only 1 unique peptide in all biological replicates.

TABLE 6
Number of proteins detected at higher
levels using summary cell-associated spectral
counts* in H1, Beijing, and both MTB rpoB
mutant strains. WT = wild-type

| Fold increased | | | |
|---|---|---|---|
| detection | H1 | Beijing | Both |
| >2 & <4 | 193 | 201 | 51 |
| >4 & <6 | 54 | 63 | 10 |
| >6 & <8 | 14 | 22 | 3 |
| >8 & <10 | 6 | 15 | 0 |
| >10 | 4 | 10 | 1 |
| Detected in | 134 | 131 | 25 |
| mutant but not | | | |
| WT | | | |

*Summary of spectral counts correspond to the sum of the mean of 3 biological replicates for CYT, CW and MEM fractions for each isolate.
Fold change values are rounded upwards at 0.5.
WT = wild-type MTB.

TABLE 7A
Summary spectral counts for cell-associated proteins upregulated by 2-fold or greater
in rpoB-mutant MTB isolates

| | CW + CYT + MEM Haarlem isolate | | | | CW + CYT + MM Beijing isolate | | | | |
|---|---|---|---|---|---|---|---|---|---|
| | rpoB | rpoB | | | rpoB | rpoB | | | Minimum |
| RV | S531L | wild- | Fold | P | H526D | wild- | Fold | P | Fold |
| number | mutant | type | change | value | mutant | type | change | value | Change |
| Rv0176 | 3.80 | 0.00 | 100.00 | 0.11 | 1.40 | 0.00 | 100.00 | 0.00 | 100.00 |
| Rv0186 | 3.21 | 0.00 | 100.00 | 0.14 | 2.00 | 0.00 | 100.00 | 0.10 | 100.00 |
| Rv0857 | 0.85 | 0.00 | 100.00 | 0.09 | 0.94 | 0.00 | 100.00 | 0.09 | 100.00 |
| Rv0872c | 0.34 | 0.00 | 100.00 | 0.21 | 0.60 | 0.00 | 100.00 | 0.21 | 100.00 |
| Rv0897c | 0.75 | 0.00 | 100.00 | 0.21 | 0.50 | 0.00 | 100.00 | 0.21 | 100.00 |
| Rv1087 | 0.42 | 0.00 | 100.00 | 0.21 | 0.50 | 0.00 | 100.00 | 0.21 | 100.00 |
| Rv1091 | 1.25 | 0.00 | 100.00 | 0.10 | 0.40 | 0.00 | 100.00 | 0.21 | 100.00 |
| Rv1173 | 0.42 | 0.00 | 100.00 | 0.21 | 2.34 | 0.00 | 100.00 | 0.21 | 100.00 |
| Rv1563c | 1.05 | 0.00 | 100.00 | 0.09 | 0.60 | 0.00 | 100.00 | 0.21 | 100.00 |
| Rv1710 | 0.49 | 0.00 | 100.00 | 0.21 | 0.94 | 0.00 | 100.00 | 0.21 | 100.00 |
| Rv1780 | 0.43 | 0.00 | 100.00 | 0.21 | 0.91 | 0.00 | 100.00 | 0.21 | 100.00 |
| Rv1812c | 0.87 | 0.00 | 100.00 | 0.21 | 3.64 | 0.00 | 100.00 | 0.14 | 100.00 |
| Rv1819c | 0.81 | 0.00 | 100.00 | 0.09 | 0.45 | 0.00 | 100.00 | 0.21 | 100.00 |
| Rv2046 | 0.56 | 0.00 | 100.00 | 0.21 | 0.60 | 0.00 | 100.00 | 0.21 | 100.00 |
| Rv2090 | 0.43 | 0.00 | 100.00 | 0.21 | 0.45 | 0.00 | 100.00 | 0.21 | 100.00 |
| Rv2232 | 0.42 | 0.00 | 100.00 | 0.21 | 0.45 | 0.00 | 100.00 | 0.21 | 100.00 |
| Rv2326c | 3.71 | 0.00 | 100.00 | 0.17 | 2.05 | 0.00 | 100.00 | 0.03 | 100.00 |
| Rv2561 | 0.75 | 0.00 | 100.00 | 0.21 | 2.00 | 0.00 | 100.00 | 0.10 | 100.00 |
| Rv2778c | 1.66 | 0.00 | 100.00 | 0.21 | 0.46 | 0.00 | 100.00 | 0.21 | 100.00 |
| Rv2787 | 0.88 | 0.00 | 100.00 | 0.21 | 1.10 | 0.00 | 100.00 | 0.09 | 100.00 |
| Rv2913c | 0.49 | 0.00 | 100.00 | 0.21 | 0.60 | 0.00 | 100.00 | 0.21 | 100.00 |
| Rv3049c | 0.56 | 0.00 | 100.00 | 0.21 | 2.14 | 0.00 | 100.00 | 0.09 | 100.00 |
| Rv3215 | 2.04 | 0.00 | 100.00 | 0.09 | 1.40 | 0.00 | 100.00 | 0.10 | 100.00 |
| Rv3405c | 1.25 | 0.00 | 100.00 | 0.21 | 0.45 | 0.00 | 100.00 | 0.21 | 100.00 |
| Rv3508 | 2.48 | 0.00 | 100.00 | 0.14 | 0.50 | 0.00 | 100.00 | 0.21 | 100.00 |
| Rv0975c | 0.49 | 0.00 | 100.00 | 0.21 | 4.80 | 0.36 | 13.17 | 0.12 | 13.17 |
| Rv0799c | 5.79 | 0.73 | 7.93 | 0.16 | 4.20 | 0.48 | 8.79 | 0.23 | 7.93 |
| Rv1188 | 0.85 | 0.00 | 100.00 | 0.09 | 2.76 | 0.45 | 6.08 | 0.04 | 6.08 |
| Rv2413c | 4.71 | 0.82 | 5.75 | 0.00 | 3.17 | 0.45 | 6.98 | 0.12 | 5.75 |
| Rv1327c | 12.92 | 2.41 | 5.36 | 0.18 | 3.16 | 0.48 | 6.62 | 0.02 | 5.36 |
| Rv1215c | 3.13 | 0.64 | 4.91 | 0.15 | 0.94 | 0.00 | 100.00 | 0.21 | 4.91 |
| Rv2936 | 3.83 | 0.00 | 100.00 | 0.06 | 6.55 | 1.41 | 4.65 | 0.00 | 4.65 |
| Rv1865c | 1.55 | 0.00 | 100.00 | 0.13 | 3.98 | 0.93 | 4.27 | 0.09 | 4.27 |
| Rv0139 | 4.63 | 1.10 | 4.21 | 0.03 | 1.99 | 0.45 | 4.38 | 0.18 | 4.21 |
| Rv3062 | 3.40 | 0.82 | 4.15 | 0.06 | 1.85 | 0.45 | 4.07 | 0.04 | 4.07 |
| Rv2153c | 0.42 | 0.00 | 100.00 | 0.21 | 1.88 | 0.48 | 3.95 | 0.27 | 3.95 |
| Rv3870 | 3.83 | 0.00 | 100.00 | 0.04 | 1.82 | 0.48 | 3.81 | 0.19 | 3.81 |
| Rv0546c | 1.07 | 0.00 | 100.00 | 0.11 | 3.85 | 1.05 | 3.68 | 0.01 | 3.68 |
| Rv2933 | 17.92 | 4.98 | 3.60 | 0.01 | 66.26 | 6.48 | 10.23 | 0.05 | 3.60 |
| Rv2024c | 7.47 | 2.16 | 3.45 | 0.04 | 4.14 | 0.45 | 9.10 | 0.07 | 3.45 |
| Rv2554c | 1.30 | 0.00 | 100.00 | 0.21 | 2.13 | 0.64 | 3.31 | 0.17 | 3.31 |
| Rv0180c | 2.41 | 0.74 | 3.27 | 0.23 | 3.65 | 0.71 | 5.13 | 0.08 | 3.27 |
| Rv3290c | 5.05 | 1.63 | 3.09 | 0.01 | 1.45 | 0.48 | 3.04 | 0.20 | 3.04 |
| Rv3223c | 1.01 | 0.33 | 3.03 | 0.19 | 9.57 | 1.94 | 4.94 | 0.10 | 3.03 |
| Rv3794 | 1.88 | 0.64 | 2.95 | 0.09 | 2.19 | 0.71 | 3.09 | 0.08 | 2.95 |
| Rv0638 | 1.43 | 0.37 | 3.88 | 0.17 | 3.93 | 1.37 | 2.88 | 0.12 | 2.88 |
| Rv1391 | 20.88 | 5.57 | 3.75 | 0.14 | 11.34 | 3.98 | 2.85 | 0.15 | 2.85 |
| Rv1638 | 4.03 | 0.37 | 10.94 | 0.07 | 14.15 | 5.15 | 2.75 | 0.03 | 2.75 |
| Rv1701 | 4.47 | 1.64 | 2.73 | 0.03 | 4.25 | 1.43 | 2.97 | 0.09 | 2.73 |
| Rv2858c | 4.51 | 1.71 | 2.65 | 0.08 | 7.02 | 2.44 | 2.87 | 0.07 | 2.65 |
| Rv2334 | 7.73 | 2.15 | 3.59 | 0.03 | 8.06 | 3.09 | 2.61 | 0.03 | 2.61 |
| Rv1882c | 4.66 | 1.80 | 2.59 | 0.16 | 1.44 | 0.00 | 100.00 | 0.12 | 2.59 |
| Rv3661 | 20.61 | 5.48 | 3.76 | 0.01 | 19.65 | 7.67 | 2.56 | 0.04 | 2.56 |
| Rv3498c | 10.24 | 4.02 | 2.55 | 0.10 | 8.60 | 1.39 | 6.20 | 0.08 | 2.55 |
| Rv2131c | 16.32 | 3.38 | 4.82 | 0.04 | 31.03 | 12.30 | 2.52 | 0.00 | 2.52 |
| Rv3075c | 2.79 | 1.12 | 2.50 | 0.14 | 4.60 | 0.99 | 4.66 | 0.03 | 2.50 |
| Rv1527c | 6.79 | 1.62 | 4.19 | 0.13 | 14.57 | 5.89 | 2.47 | 0.01 | 2.47 |
| Rv2203 | 1.68 | 0.37 | 4.57 | 0.18 | 4.99 | 2.03 | 2.46 | 0.20 | 2.46 |
| Rv3915 | 3.81 | 1.56 | 2.45 | 0.18 | 9.70 | 3.68 | 2.63 | 0.08 | 2.45 |
| Rv0383c | 1.72 | 0.72 | 2.41 | 0.15 | 1.35 | 0.00 | 100.00 | 0.11 | 2.41 |
| Rv2927c | 7.39 | 2.24 | 3.30 | 0.06 | 6.17 | 2.60 | 2.38 | 0.08 | 2.38 |
| Rv2931 | 16.45 | 0.72 | 23.00 | 0.05 | 71.16 | 30.15 | 2.36 | 0.06 | 2.36 |
| Rv0409 | 1.36 | 0.37 | 3.71 | 0.17 | 4.24 | 1.80 | 2.36 | 0.13 | 2.36 |
| Rv2737c | 1.72 | 0.73 | 2.36 | 0.16 | 4.02 | 1.55 | 2.59 | 0.05 | 2.36 |
| Rv3512 | 4.69 | 1.64 | 2.85 | 0.14 | 2.40 | 1.02 | 2.35 | 0.26 | 2.35 |
| Rv3586 | 11.10 | 4.74 | 2.34 | 0.01 | 8.24 | 3.25 | 2.54 | 0.17 | 2.34 |
| Rv0251c | 3.80 | 1.64 | 2.32 | 0.23 | 26.15 | 8.81 | 2.97 | 0.11 | 2.32 |
| Rv2739c | 0.85 | 0.37 | 2.31 | 0.22 | 1.40 | 0.00 | 100.00 | 0.00 | 2.31 |
| Rv2166c | 2.44 | 1.06 | 2.31 | 0.31 | 6.57 | 1.14 | 5.76 | 0.19 | 2.31 |
| Rv2074 | 5.29 | 1.72 | 3.07 | 0.11 | 9.95 | 4.42 | 2.25 | 0.06 | 2.25 |
| Rv3792 | 1.74 | 0.00 | 100.00 | 0.09 | 0.99 | 0.45 | 2.18 | 0.33 | 2.18 |
| Rv3423c | 7.11 | 3.02 | 2.36 | 0.08 | 16.30 | 7.47 | 2.18 | 0.08 | 2.18 |
| Rv2921c | 7.59 | 2.19 | 3.47 | 0.04 | 8.48 | 3.90 | 2.18 | 0.11 | 2.18 |
| Rv0496 | 2.54 | 1.17 | 2.17 | 0.20 | 9.73 | 2.84 | 3.42 | 0.09 | 2.17 |
| Rv3818 | 4.94 | 1.18 | 4.19 | 0.06 | 3.76 | 1.74 | 2.16 | 0.10 | 2.16 |
| Rv2606c | 28.51 | 13.08 | 2.18 | 0.03 | 32.39 | 15.02 | 2.16 | 0.01 | 2.16 |
| Rv3281 | 2.11 | 0.33 | 6.35 | 0.11 | 2.23 | 1.03 | 2.16 | 0.29 | 2.16 |
| Rv2110c | 20.00 | 9.45 | 2.12 | 0.11 | 9.64 | 4.15 | 2.32 | 0.04 | 2.12 |
| Rv2062c | 4.87 | 0.70 | 6.96 | 0.03 | 1.35 | 0.64 | 2.11 | 0.26 | 2.11 |
| Rv1260 | 1.45 | 0.69 | 2.10 | 0.21 | 1.13 | 0.34 | 3.32 | 0.18 | 2.10 |
| Rv3273 | 28.11 | 6.95 | 4.04 | 0.05 | 52.01 | 24.80 | 2.10 | 0.00 | 2.10 |
| Rv0129c | 9.92 | 4.74 | 2.09 | 0.02 | 4.29 | 0.93 | 4.60 | 0.14 | 2.09 |
| Rv2586c | 1.70 | 0.82 | 2.08 | 0.10 | 1.44 | 0.64 | 2.24 | 0.25 | 2.08 |
| Rv1769 | 1.11 | 0.00 | 100.00 | 0.21 | 2.94 | 1.42 | 2.07 | 0.20 | 2.07 |
| Rv0576 | 6.20 | 2.81 | 2.20 | 0.05 | 4.39 | 2.12 | 2.07 | 0.05 | 2.07 |
| Rv2289 | 16.92 | 7.57 | 2.24 | 0.02 | 22.59 | 11.00 | 2.05 | 0.09 | 2.05 |
| Rv2192c | 0.75 | 0.37 | 2.05 | 0.34 | 3.25 | 0.00 | 100.00 | 0.01 | 2.05 |
| Rv3777 | 4.25 | 2.08 | 2.04 | 0.11 | 2.68 | 0.36 | 7.34 | 0.12 | 2.04 |
| Rv1340 | 11.73 | 3.78 | 3.11 | 0.00 | 8.28 | 4.08 | 2.03 | 0.02 | 2.03 |
| Rv2184c | 5.94 | 2.94 | 2.02 | 0.27 | 6.00 | 1.10 | 5.46 | 0.09 | 2.02 |

TABLE 7B
Summary spectral counts for cell-associated proteins upregulated by 2-fold or
greater in rpoB-mutant MTB isolates

| | CW + CYT + MEM Haarlem isolate | | | | | | CW + CYT + MEM Beijing isolate | | | | | |
|---|---|---|---|---|---|---|---|---|---|---|---|---|
| | Replicate spectral counts | | | | | | Replicate spectral counts | | | | | |
| RV | rpoB S531L mutant | | | rpoB wild-type | | | rpoB H526D mutant | | | rpoB wild-type | | |
| number | 1.00 | 2.00 | 3.00 | 1.00 | 2.00 | 3.00 | 1.00 | 2.00 | 3.00 | 1.00 | 2.00 | 3.00 |
| Rv0176 | 0.00 | 3.91 | 7.49 | 0.00 | 0.00 | 0.00 | 1.35 | 1.37 | 1.49 | 0.00 | 0.00 | 0.00 |
| Rv0186 | 0.00 | 7.38 | 2.26 | 0.00 | 0.00 | 0.00 | 3.60 | 0.00 | 2.41 | 0.00 | 0.00 | 0.00 |
| Rv0857 | 1.31 | 0.00 | 1.25 | 0.00 | 0.00 | 0.00 | 1.35 | 0.00 | 1.49 | 0.00 | 0.00 | 0.00 |
| Rv0872c | 0.00 | 0.00 | 1.01 | 0.00 | 0.00 | 0.00 | 1.80 | 0.00 | 0.00 | 0.00 | 0.00 | 0.00 |
| Rv0897c | 0.00 | 0.00 | 2.26 | 0.00 | 0.00 | 0.00 | 0.00 | 0.00 | 1.49 | 0.00 | 0.00 | 0.00 |
| Rv1087 | 0.00 | 0.00 | 1.25 | 0.00 | 0.00 | 0.00 | 0.00 | 0.00 | 1.49 | 0.00 | 0.00 | 0.00 |
| Rv1091 | 0.00 | 1.48 | 2.26 | 0.00 | 0.00 | 0.00 | 0.00 | 0.00 | 1.19 | 0.00 | 0.00 | 0.00 |
| Rv1173 | 0.00 | 0.00 | 1.25 | 0.00 | 0.00 | 0.00 | 0.00 | 7.03 | 0.00 | 0.00 | 0.00 | 0.00 |
| Rv1563c | 1.67 | 1.48 | 0.00 | 0.00 | 0.00 | 0.00 | 1.80 | 0.00 | 0.00 | 0.00 | 0.00 | 0.00 |
| Rv1710 | 0.00 | 1.48 | 0.00 | 0.00 | 0.00 | 0.00 | 0.00 | 2.83 | 0.00 | 0.00 | 0.00 | 0.00 |
| Rv1780 | 0.00 | 1.30 | 0.00 | 0.00 | 0.00 | 0.00 | 0.00 | 2.74 | 0.00 | 0.00 | 0.00 | 0.00 |
| Rv1812c | 0.00 | 2.60 | 0.00 | 0.00 | 0.00 | 0.00 | 2.69 | 8.23 | 0.00 | 0.00 | 0.00 | 0.00 |
| Rv1819c | 1.17 | 0.00 | 1.25 | 0.00 | 0.00 | 0.00 | 1.35 | 0.00 | 0.00 | 0.00 | 0.00 | 0.00 |
| Rv2046 | 1.67 | 0.00 | 0.00 | 0.00 | 0.00 | 0.00 | 1.80 | 0.00 | 0.00 | 0.00 | 0.00 | 0.00 |
| Rv2090 | 0.00 | 1.30 | 0.00 | 0.00 | 0.00 | 0.00 | 1.35 | 0.00 | 0.00 | 0.00 | 0.00 | 0.00 |
| Rv2232 | 0.00 | 0.00 | 1.25 | 0.00 | 0.00 | 0.00 | 1.35 | 0.00 | 0.00 | 0.00 | 0.00 | 0.00 |
| Rv2326c | 9.66 | 1.48 | 0.00 | 0.00 | 0.00 | 0.00 | 1.80 | 1.37 | 2.98 | 0.00 | 0.00 | 0.00 |
| Rv2561 | 0.00 | 0.00 | 2.26 | 0.00 | 0.00 | 0.00 | 3.60 | 0.00 | 2.41 | 0.00 | 0.00 | 0.00 |
| Rv2778c | 0.00 | 0.00 | 4.99 | 0.00 | 0.00 | 0.00 | 0.00 | 1.37 | 0.00 | 0.00 | 0.00 | 0.00 |
| Rv2787 | 2.63 | 0.00 | 0.00 | 0.00 | 0.00 | 0.00 | 1.80 | 0.00 | 1.49 | 0.00 | 0.00 | 0.00 |
| Rv2913c | 0.00 | 1.48 | 0.00 | 0.00 | 0.00 | 0.00 | 1.80 | 0.00 | 0.00 | 0.00 | 0.00 | 0.00 |
| Rv3049c | 1.67 | 0.00 | 0.00 | 0.00 | 0.00 | 0.00 | 3.60 | 2.83 | 0.00 | 0.00 | 0.00 | 0.00 |
| Rv3215 | 3.34 | 2.78 | 0.00 | 0.00 | 0.00 | 0.00 | 1.80 | 0.00 | 2.41 | 0.00 | 0.00 | 0.00 |
| Rv3405c | 0.00 | 0.00 | 3.75 | 0.00 | 0.00 | 0.00 | 1.35 | 0.00 | 0.00 | 0.00 | 0.00 | 0.00 |
| Rv3508 | 1.67 | 0.00 | 5.77 | 0.00 | 0.00 | 0.00 | 0.00 | 0.00 | 1.49 | 0.00 | 0.00 | 0.00 |
| Rv0975c | 0.00 | 1.48 | 0.00 | 0.00 | 0.00 | 0.00 | 0.00 | 4.81 | 9.60 | 0.00 | 1.09 | 0.00 |
| Rv0799c | 13.35 | 2.78 | 1.25 | 2.19 | 0.00 | 0.00 | 12.59 | 0.00 | 0.00 | 0.00 | 1.43 | 0.00 |
| Rv1188 | 1.31 | 0.00 | 1.25 | 0.00 | 0.00 | 0.00 | 2.69 | 4.11 | 1.49 | 1.36 | 0.00 | 0.00 |
| Rv2413c | 3.94 | 5.21 | 4.99 | 1.10 | 0.00 | 1.36 | 4.04 | 5.49 | 0.00 | 1.36 | 0.00 | 0.00 |
| Rv1327c | 1.17 | 7.20 | 30.40 | 2.13 | 1.12 | 3.99 | 1.80 | 4.11 | 3.56 | 0.00 | 1.43 | 0.00 |
| Rv1215c | 6.67 | 1.48 | 1.25 | 0.00 | 1.91 | 0.00 | 0.00 | 2.83 | 0.00 | 0.00 | 0.00 | 0.00 |
| Rv2936 | 3.94 | 1.30 | 6.24 | 0.00 | 0.00 | 0.00 | 6.73 | 5.49 | 7.45 | 1.36 | 2.86 | 0.00 |
| Rv1865c | 3.34 | 1.30 | 0.00 | 0.00 | 0.00 | 0.00 | 3.60 | 6.86 | 1.49 | 1.36 | 1.43 | 0.00 |
| Rv0139 | 4.65 | 5.73 | 3.51 | 3.30 | 0.00 | 0.00 | 4.49 | 0.00 | 1.49 | 1.36 | 0.00 | 0.00 |
| Rv3062 | 1.31 | 3.91 | 4.99 | 1.10 | 0.00 | 1.36 | 2.69 | 1.37 | 1.49 | 1.36 | 0.00 | 0.00 |
| Rv2153c | 0.00 | 0.00 | 1.25 | 0.00 | 0.00 | 0.00 | 0.00 | 5.65 | 0.00 | 0.00 | 1.43 | 0.00 |
| Rv3870 | 2.63 | 2.60 | 6.24 | 0.00 | 0.00 | 0.00 | 1.35 | 4.11 | 0.00 | 0.00 | 1.43 | 0.00 |
| Rv0546c | 1.17 | 0.00 | 2.03 | 0.00 | 0.00 | 0.00 | 4.38 | 3.61 | 3.56 | 0.00 | 1.09 | 2.05 |
| Rv2933 | 21.74 | 15.79 | 16.23 | 4.42 | 6.44 | 4.07 | 49.40 | 106.62 | 42.76 | 10.59 | 1.43 | 7.41 |
| Rv2024c | 5.61 | 5.73 | 11.07 | 1.10 | 3.03 | 2.35 | 1.35 | 4.20 | 6.88 | 1.36 | 0.00 | 0.00 |
| Rv2554c | 0.00 | 3.91 | 0.00 | 0.00 | 0.00 | 0.00 | 2.44 | 3.95 | 0.00 | 0.00 | 0.00 | 1.93 |
| Rv0180c | 5.97 | 0.00 | 1.25 | 2.21 | 0.00 | 0.00 | 1.80 | 2.83 | 6.31 | 0.00 | 0.00 | 2.13 |
| Rv3290c | 3.94 | 6.20 | 4.99 | 1.06 | 1.12 | 2.71 | 0.00 | 1.37 | 2.98 | 0.00 | 1.43 | 0.00 |
| Rv3223c | 0.00 | 1.00 | 2.03 | 0.00 | 0.00 | 1.00 | 3.79 | 17.51 | 7.42 | 4.79 | 0.00 | 1.02 |
| Rv3794 | 1.67 | 1.48 | 2.50 | 0.00 | 1.91 | 0.00 | 1.35 | 2.83 | 2.41 | 0.00 | 0.00 | 2.13 |
| Rv0638 | 2.98 | 1.30 | 0.00 | 1.10 | 0.00 | 0.00 | 1.80 | 7.03 | 2.98 | 0.00 | 1.97 | 2.13 |
| Rv1391 | 6.93 | 14.84 | 40.86 | 9.92 | 0.00 | 6.79 | 20.18 | 12.34 | 1.49 | 4.09 | 5.73 | 2.13 |
| Rv1638 | 6.93 | 3.91 | 1.25 | 1.10 | 0.00 | 0.00 | 16.61 | 9.69 | 16.17 | 2.73 | 2.86 | 9.85 |
| Rv1701 | 4.30 | 5.38 | 3.75 | 2.21 | 0.00 | 2.71 | 2.69 | 4.11 | 5.96 | 0.00 | 4.30 | 0.00 |
| Rv2858c | 7.03 | 3.47 | 3.04 | 2.13 | 0.00 | 2.99 | 5.48 | 10.82 | 4.75 | 1.03 | 1.09 | 5.20 |
| Rv2334 | 9.20 | 7.89 | 6.08 | 0.00 | 1.12 | 5.35 | 5.98 | 8.42 | 9.80 | 2.40 | 1.09 | 5.79 |
| Rv1882c | 2.63 | 2.60 | 8.74 | 1.10 | 4.29 | 0.00 | 1.35 | 0.00 | 2.98 | 0.00 | 0.00 | 0.00 |
| Rv3661 | 26.50 | 16.32 | 19.03 | 11.02 | 0.00 | 5.43 | 25.14 | 22.11 | 11.70 | 10.25 | 4.83 | 7.92 |
| Rv3498c | 11.83 | 3.91 | 14.98 | 6.62 | 0.00 | 5.43 | 4.04 | 6.86 | 14.90 | 2.73 | 1.43 | 0.00 |
| Rv2131c | 7.64 | 20.57 | 20.75 | 5.52 | 1.91 | 2.71 | 30.97 | 36.08 | 26.02 | 8.88 | 14.50 | 13.51 |
| Rv3075c | 2.34 | 3.99 | 2.03 | 0.00 | 3.35 | 0.00 | 2.44 | 6.01 | 5.35 | 1.03 | 0.00 | 1.93 |
| Rv1527c | 2.98 | 3.91 | 13.50 | 0.00 | 2.15 | 2.71 | 14.38 | 18.00 | 11.35 | 5.82 | 3.94 | 7.92 |
| Rv2203 | 0.00 | 1.30 | 3.75 | 1.10 | 0.00 | 0.00 | 5.38 | 9.60 | 0.00 | 2.73 | 1.43 | 1.93 |
| Rv3915 | 2.63 | 1.30 | 7.49 | 3.31 | 0.00 | 1.36 | 14.83 | 8.31 | 5.96 | 8.19 | 2.86 | 0.00 |
| Rv0383c | 1.31 | 2.60 | 1.25 | 0.00 | 2.15 | 0.00 | 2.69 | 1.37 | 0.00 | 0.00 | 0.00 | 0.00 |
| Rv2927c | 3.94 | 10.89 | 7.33 | 2.21 | 2.15 | 2.35 | 8.92 | 3.95 | 5.66 | 2.40 | 5.39 | 0.00 |
| Rv2931 | 27.71 | 11.89 | 9.75 | 0.00 | 2.15 | 0.00 | 56.14 | 90.50 | 66.86 | 58.16 | 1.43 | 30.87 |
| Rv0409 | 1.31 | 2.78 | 0.00 | 1.10 | 0.00 | 0.00 | 1.80 | 7.03 | 3.90 | 3.42 | 1.97 | 0.00 |
| Rv2737c | 1.31 | 2.60 | 1.25 | 2.19 | 0.00 | 0.00 | 4.04 | 4.11 | 3.90 | 2.73 | 0.00 | 1.93 |
| Rv3512 | 8.81 | 2.78 | 2.50 | 1.10 | 1.12 | 2.71 | 1.80 | 5.40 | 0.00 | 0.00 | 0.00 | 3.07 |
| Rv3586 | 13.15 | 10.41 | 9.75 | 5.52 | 1.91 | 6.79 | 8.07 | 15.17 | 1.49 | 5.46 | 4.30 | 0.00 |
| Rv0251c | 7.89 | 0.00 | 3.51 | 2.21 | 0.00 | 2.71 | 29.17 | 41.82 | 7.45 | 15.01 | 7.16 | 4.26 |
| Rv2739c | 0.00 | 1.30 | 1.25 | 1.10 | 0.00 | 0.00 | 1.35 | 1.37 | 1.49 | 0.00 | 0.00 | 0.00 |
| Rv2166c | 0.00 | 0.00 | 7.33 | 1.06 | 1.12 | 1.00 | 3.60 | 16.12 | 0.00 | 3.42 | 0.00 | 0.00 |
| Rv2074 | 3.52 | 2.99 | 9.36 | 2.17 | 0.00 | 2.99 | 12.70 | 11.50 | 5.66 | 3.10 | 4.38 | 5.79 |
| Rv3792 | 2.63 | 2.60 | 0.00 | 0.00 | 0.00 | 0.00 | 0.00 | 0.00 | 2.98 | 1.36 | 0.00 | 0.00 |
| Rv3423c | 6.32 | 4.25 | 10.76 | 4.42 | 1.91 | 2.71 | 20.68 | 9.85 | 18.36 | 4.09 | 3.40 | 14.91 |
| Rv2921c | 5.97 | 5.55 | 11.24 | 3.30 | 1.91 | 1.36 | 4.04 | 8.23 | 13.19 | 5.46 | 4.30 | 1.93 |
| Rv0496 | 1.31 | 1.30 | 4.99 | 0.00 | 2.15 | 1.36 | 16.14 | 4.11 | 8.94 | 1.36 | 7.16 | 0.00 |
| Rv3818 | 5.26 | 2.30 | 7.26 | 1.06 | 1.12 | 1.36 | 2.69 | 4.11 | 4.47 | 1.36 | 0.00 | 3.86 |
| Rv2606c | 22.55 | 37.96 | 25.03 | 10.68 | 18.08 | 10.47 | 29.49 | 38.15 | 29.54 | 17.46 | 14.45 | 13.15 |
| Rv3281 | 0.00 | 3.30 | 3.04 | 0.00 | 0.00 | 1.00 | 5.48 | 1.20 | 0.00 | 3.10 | 0.00 | 0.00 |
| Rv2110c | 31.80 | 10.97 | 17.23 | 9.58 | 7.81 | 10.97 | 10.95 | 12.03 | 5.93 | 4.13 | 2.19 | 6.14 |
| Rv2062c | 6.57 | 5.55 | 2.50 | 1.10 | 0.00 | 1.00 | 2.69 | 1.37 | 0.00 | 0.00 | 0.00 | 1.93 |
| Rv1260 | 2.34 | 1.99 | 0.00 | 1.06 | 0.00 | 1.00 | 2.19 | 1.20 | 0.00 | 0.00 | 0.00 | 1.02 |
| Rv3273 | 12.54 | 35.76 | 36.03 | 11.04 | 5.74 | 4.07 | 52.95 | 56.65 | 46.44 | 32.08 | 16.83 | 25.48 |
| Rv0129c | 8.92 | 12.10 | 8.74 | 7.61 | 3.26 | 3.35 | 7.82 | 0.00 | 5.05 | 1.36 | 1.43 | 0.00 |
| Rv2586c | 1.31 | 1.30 | 2.50 | 1.10 | 0.00 | 1.36 | 1.35 | 0.00 | 2.98 | 0.00 | 0.00 | 1.93 |
| Rv1769 | 3.34 | 0.00 | 0.00 | 0.00 | 0.00 | 0.00 | 3.60 | 2.83 | 2.41 | 0.00 | 0.00 | 4.26 |
| Rv0576 | 5.19 | 5.29 | 8.11 | 5.32 | 1.12 | 2.00 | 3.29 | 6.01 | 3.86 | 1.03 | 3.28 | 2.05 |
| Rv2289 | 16.59 | 20.92 | 13.26 | 12.09 | 3.83 | 6.79 | 21.55 | 23.74 | 22.48 | 9.55 | 21.31 | 2.13 |
| Rv2192c | 0.00 | 0.00 | 2.26 | 1.10 | 0.00 | 0.00 | 3.14 | 4.20 | 2.41 | 0.00 | 0.00 | 0.00 |
| Rv3777 | 4.69 | 2.99 | 5.07 | 4.26 | 0.00 | 2.00 | 3.29 | 0.00 | 4.75 | 0.00 | 1.09 | 0.00 |
| Rv1340 | 9.48 | 12.15 | 13.57 | 5.38 | 2.23 | 3.71 | 8.02 | 6.60 | 10.21 | 5.46 | 2.53 | 4.26 |
| Rv2184c | 6.57 | 0.00 | 11.24 | 8.83 | 0.00 | 0.00 | 4.04 | 10.97 | 2.98 | 1.36 | 0.00 | 1.93 |

Gene Set Enrichment Analysis of Proteins Upregulated in Both rpoB Mutants

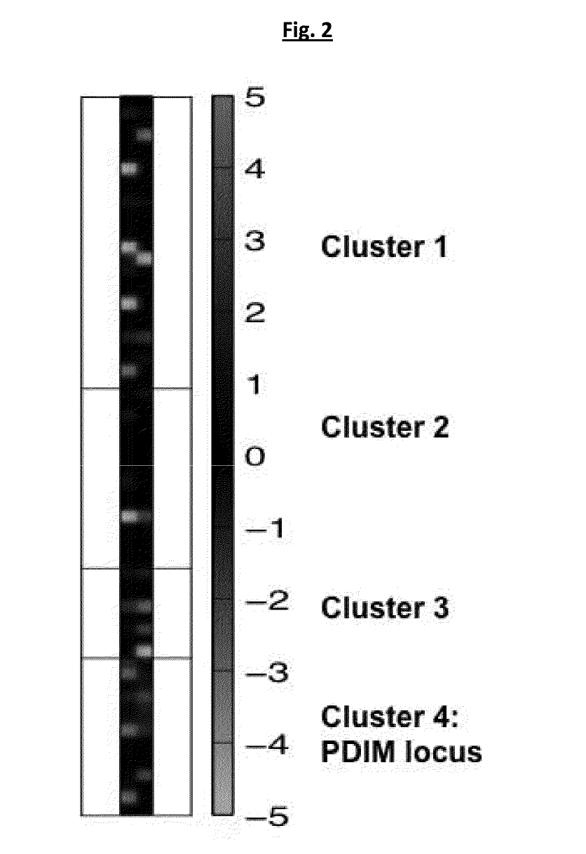

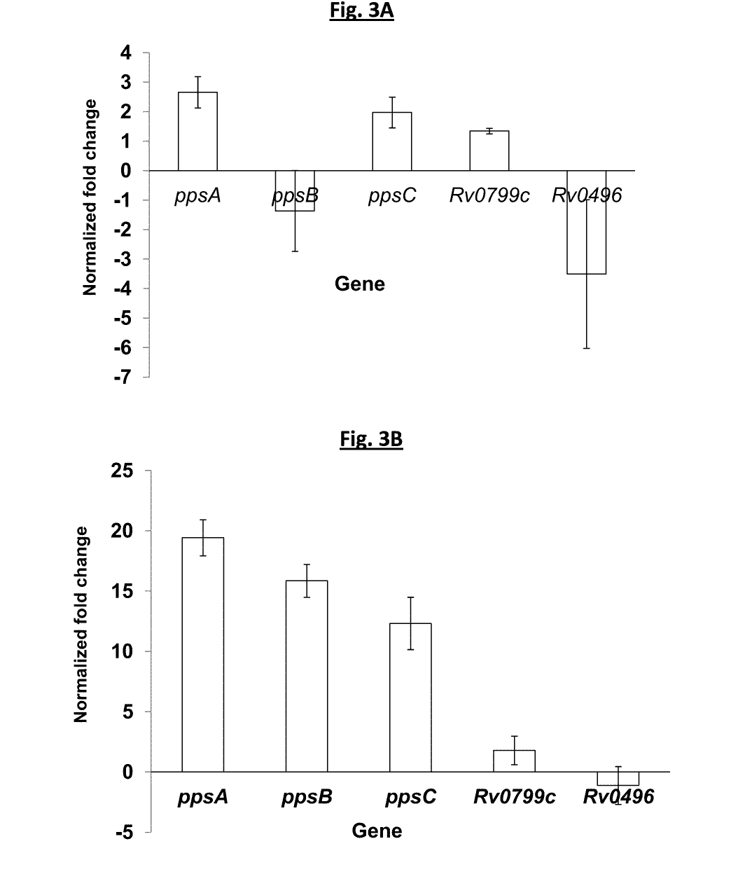

The distribution of functional classes among genes upregulated >2 fold in both rpoB mutants vs. genes not upregulated in both mutants was not statistically significantly different when comparing the proportion of genes in each Functional Categorization of Gene Products (COG) class (chi square tests all p>0.05). Among the four gene loci identified as possibly involved in natural product biosynthesis in MTB by NP.searcher, the polyketide synthase gene cluster ppsA-E and drrA was significantly upregulated in both rpoB mutants (FIG. 2).

Selected Expression of Genes in the PDIM Biosynthetic Locus in the Beijing rpoB Mutant MTB During Active Infection of Murine Macrophages

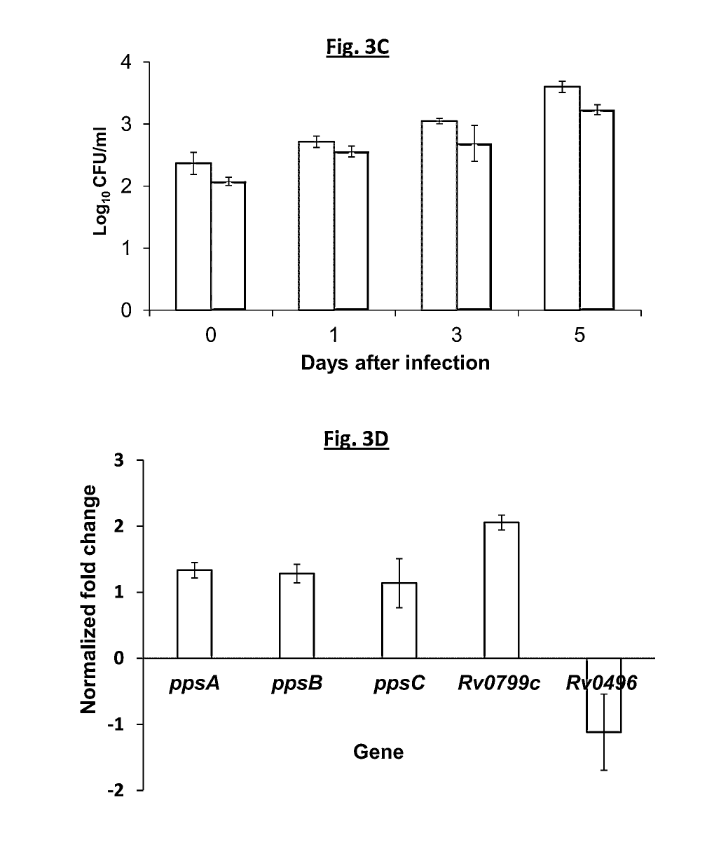

In order to determine if increased expression of proteins involved in PDIM biosynthesis correlated with increased transcription of the corresponding genes, qRT-PCR was performed. Consistent with the proteomic analysis, the ppsA gene was found to be upregulated >2-fold in the Beijing rpoB mutant relative to the parental strain (FIG. 3A). Next, using qRT-PCR, experiments were performed to determine the effect of rpoB mutation on expression levels of selected PDIM biosynthesis genes during Mtb infection of activated murine macrophages. The genes ppsA, ppsC, and ppsE were each found to have >10-fold increased expression in the rpoB mutant relative to the rifampicin-sensitive parent strain, while expression of the control gene Rv0496 was not different between the two strains (FIG. 3B). A comparison of upregulation in FIGS. 3A and 3B indicates that the upregulation was more striking in activated macrophages than when grown in nutrient rich media. Consistent with the proteomic data, Rv0799c, which encodes a conserved hypothetical protein, also was found to be mildly upregulated in the rpoB mutant relative to the parent strain in activated murine macrophages. The rpoB mutation did not appear to confer a fitness cost as the rpoB mutant and the WT-parent strain showed nearly equivalent growth rates and survival in activated macrophages (FIG. 3C). Finally, in order to confirm that the gene expression profile observed in the rpoB mutant was attributable to the RpoB S450L mutation in the rpoB gene, a mutant strain containing the RpoB S450L mutation in the background of the laboratory strain MTB CDC1551 was experimentally generated. The genes ppsA, ppsB, ppsC, and Rv0799c were each upregulated in this RpoB S450L mutant relative to the isogenic CDC1551 wild-type strain during logarithmic growth in nutrient-rich broth (FIG. 3D).

### Metabolomics Analysis

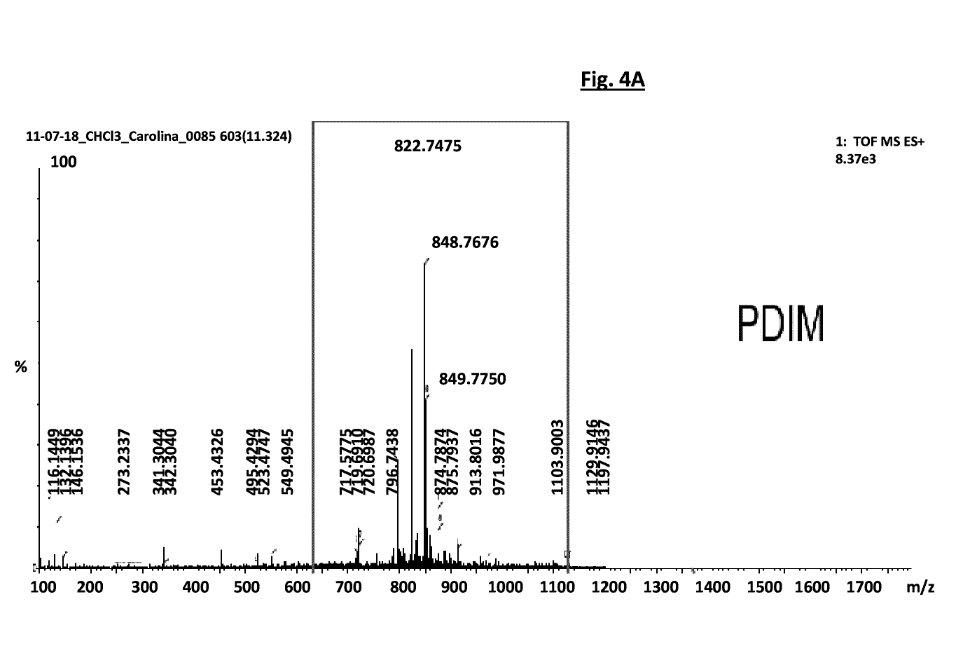

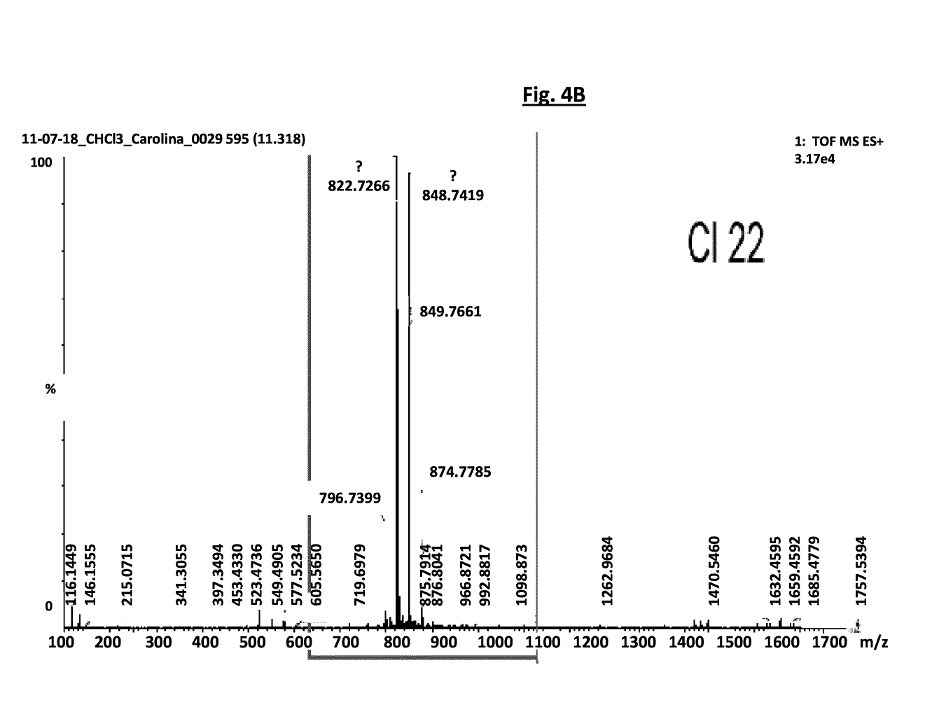

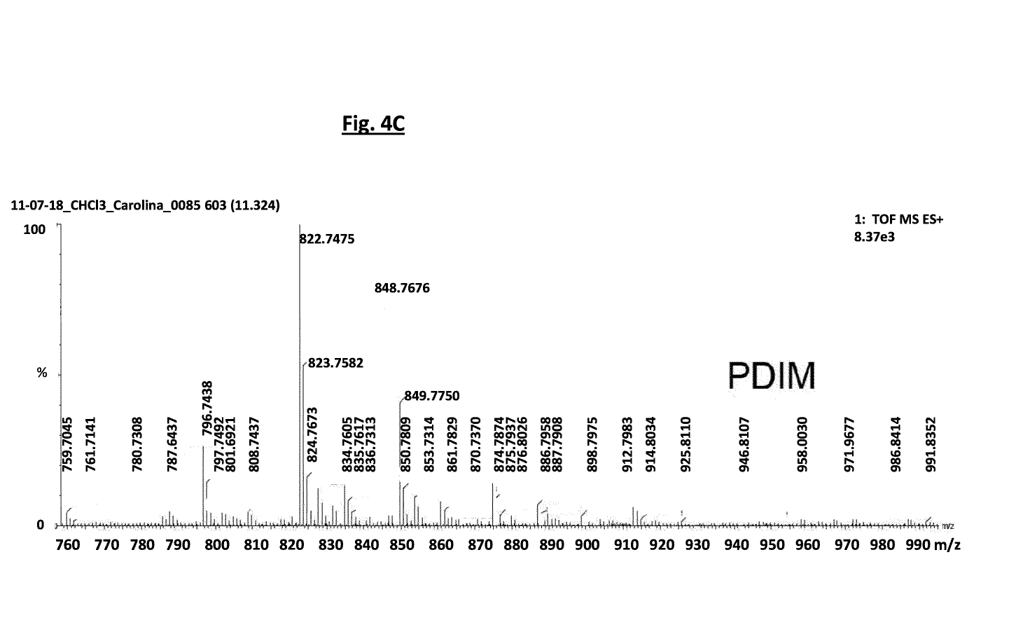

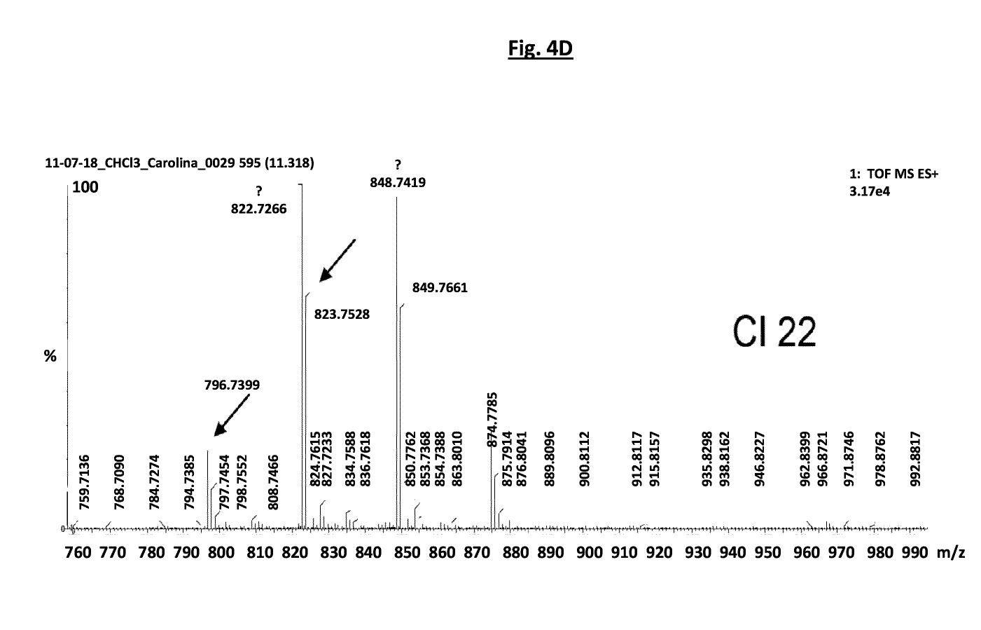

In addition to the proteomics experiments described elsewhere herein, metabolomic analyses were performed in order to determine if rpoB mutations also might lead to changes in the MTB metabolome. Because of the great complexity of the sample, two sequential extractions were performed to separate cell wall associated lipids and other non-polar metabolites from more polar metabolites. This analysis revealed 99 molecular features in the chloroform fraction that were significantly more abundant (p<0.05) in both rifampicin resistant MTB strains when compared to their susceptible pairs (Table 8). A search performed against the LipidMaps database suggested that several of these significant features correspond to diacylglycerol phosphocholines (Table 9). Since the proteomics and GSEA findings suggested that the polyketide synthase genes involved in phenolpthiocerol biosynthesis are upregulated in the rpoB mutant isolates of MTB, the chloroform extract was compared to a positive authentic PDIM standard that was obtained from MTB H37Rv and analyzed by LC-MS using identical parameters. The PDIM standard was obtained through the Tuberculosis Vaccine Testing and Research Material Contract (TVTRMC). The 797.7 m/z and 823.2 m/z signals, observed to increase in the resistant strains relative to their respective parent strains (FIG. 4 and Table 9), were also found in an authentic PDIM standard. When MS/MS analysis was performed on the 797.7 m/z ion, a fragment of 523.47 m/z and neutral loss of 274.27 was observed (data not shown). The difference in 2 Da from the expected 525.5 m/z of phthiocerol (Jain et al., 2007, Proc Natl Acad Sci USA 104:5133-8) suggests desaturation by loss of the methyl group and therefore most likely corresponds to phthiodiolone. Without wishing to be bound by any particular theory, these data are consistent with accumulation of PDIM precursors in the rifampicin-resistant isolates, likely due to elevated levels of polyketide synthase-related proteins.

TABLE 8
Molecular features of metabolites identified in the
chloroform fraction as statistically significantly more
abundant in both rpoB mutant MTB isolates compared
to both wild-type MTB parent strains. WT = wild-type

| | Haarlem | | | Beijing | | |
|---|---|---|---|---|---|---|
| | rpoB | Haarlem | p- | rpoB | Beijing | p- |
| Sample.ID | mutant | WT | value | mutant | WT | value |
| 196.1/50 | 2.538 | 1.764 | 0.042 | 5.953 | 2.870 | 0.000 |
| 151.1/103 | 4.230 | 3.489 | 0.049 | 10.999 | 4.136 | 0.000 |
| 347.1/103 | 32.074 | 19.607 | 0.012 | 1.185 | 0.361 | 0.018 |
| 285.1/104 | 14.951 | 10.984 | 0.045 | 6.260 | 2.430 | 0.000 |
| 375.2/112 | 26.466 | 17.290 | 0.028 | 28.098 | 17.240 | 0.010 |
| 475.2/116 | 8.950 | 5.422 | 0.047 | 13.404 | 8.264 | 0.049 |
| 427.3/127 | 23.465 | 12.837 | 0.047 | 41.055 | 12.880 | 0.000 |
| 546.4/128 | 7.611 | 3.308 | 0.041 | 11.188 | 3.499 | 0.000 |
| 181.1/133 | 58.782 | 40.611 | 0.042 | 7.447 | 4.986 | 0.008 |
| 594.4/177 | 14.813 | 9.528 | 0.016 | 14.680 | 7.779 | 0.002 |
| 161.1/193 | 38.213 | 26.954 | 0.018 | 50.727 | 31.302 | 0.007 |
| 189.1/193 | 46.316 | 33.655 | 0.028 | 58.327 | 36.627 | 0.007 |
| 190.1/193 | 6.849 | 4.715 | 0.027 | 8.526 | 5.944 | 0.018 |
| 217.1/193 | 39.670 | 33.480 | 0.045 | 46.407 | 31.644 | 0.005 |
| 221.2/193 | 54.931 | 39.575 | 0.026 | 72.132 | 47.362 | 0.010 |
| 281.2/193 | 74.497 | 54.810 | 0.037 | 106.317 | 66.623 | 0.013 |
| 249.2/194 | 105.222 | 74.568 | 0.017 | 146.174 | 90.825 | 0.009 |
| 508.4/222 | 5.670 | 2.779 | 0.009 | 8.796 | 1.321 | 0.002 |
| 644.5/222 | 12.170 | 5.383 | 0.043 | 19.891 | 2.795 | 0.010 |
| 971.8/302 | 12.242 | 7.113 | 0.048 | 12.539 | 4.322 | 0.013 |
| 200.1/318 | 11.598 | 4.620 | 0.043 | 6.580 | 3.204 | 0.001 |
| 215.1/318 | 217.697 | 89.613 | 0.050 | 126.643 | 62.609 | 0.001 |
| 747.6/318 | 444.030 | 189.220 | 0.048 | 243.566 | 119.076 | 0.001 |
| 819.7/318 | 74.700 | 31.109 | 0.048 | 41.029 | 25.357 | 0.015 |
| 838.7/318 | 54.247 | 17.966 | 0.046 | 26.170 | 11.222 | 0.001 |
| 591.4/342 | 31.888 | 19.977 | 0.028 | 22.243 | 12.112 | 0.001 |
| 715.1/351 | 14.101 | 10.115 | 0.017 | 11.453 | 7.054 | 0.001 |
| 810.3/351 | 4.401 | 2.606 | 0.021 | 3.352 | 1.389 | 0.002 |
| 483.3/352 | 26.860 | 20.840 | 0.039 | 19.418 | 14.853 | 0.049 |
| 832/353 | 10.908 | 8.058 | 0.002 | 5.012 | 3.517 | 0.009 |
| 775.6/371 | 8.159 | 5.026 | 0.010 | 17.874 | 11.593 | 0.009 |
| 667.5/379 | 5.531 | 3.157 | 0.026 | 10.912 | 2.695 | 0.007 |
| 420.3/409 | 2.710 | 2.001 | 0.028 | 4.936 | 1.922 | 0.011 |
| 813.3/416 | 4.682 | 3.365 | 0.030 | 2.396 | 1.258 | 0.011 |
| 571.4/445 | 20.489 | 12.628 | 0.037 | 11.210 | 6.038 | 0.025 |
| 780.6/487 | 31.377 | 20.849 | 0.044 | 126.434 | 63.609 | 0.032 |
| 804.6/495 | 221.362 | 151.519 | 0.041 | 1495.045 | 228.206 | 0.000 |
| 818.6/508 | 17.786 | 12.817 | 0.024 | 78.712 | 19.025 | 0.000 |
| 807.6/517 | 110.520 | 69.352 | 0.041 | 396.873 | 59.912 | 0.000 |
| 808.6/517 | 40.775 | 27.568 | 0.049 | 127.011 | 25.237 | 0.000 |
| 822.5/527 | 50.296 | 28.369 | 0.045 | 77.807 | 17.142 | 0.000 |
| 927.6/552 | 6.728 | 4.477 | 0.034 | 21.163 | 13.703 | 0.023 |
| 775.6/577 | 35.903 | 26.631 | 0.026 | 58.827 | 44.596 | 0.001 |
| 993.7/582 | 3.091 | 2.069 | 0.048 | 8.795 | 4.359 | 0.014 |
| 890.7/595 | 32.886 | 23.706 | 0.019 | 64.686 | 15.270 | 0.000 |
| 617.9/601 | 1.718 | 1.184 | 0.032 | 1.276 | 0.487 | 0.001 |
| 295.2/610 | 14.901 | 11.049 | 0.031 | 8.816 | 5.006 | 0.024 |
| 119.1/611 | 12.300 | 9.179 | 0.036 | 7.171 | 4.720 | 0.018 |
| 632.6/615 | 8.035 | 3.727 | 0.031 | 92.126 | 3.992 | 0.000 |
| 777.7/643 | 4.649 | 2.950 | 0.017 | 21.838 | 7.850 | 0.007 |
| 776.7/644 | 11.359 | 6.732 | 0.027 | 42.342 | 31.766 | 0.014 |
| 688.6/647 | 21.297 | 10.520 | 0.028 | 111.529 | 23.667 | 0.000 |
| 781.2/663 | 0.730 | 0.299 | 0.017 | 2.185 | 0.777 | 0.005 |
| 455.4/668 | 5.810 | 4.224 | 0.020 | 10.455 | 6.376 | 0.003 |
| 819.4/675 | 5.034 | 4.061 | 0.048 | 17.534 | 10.332 | 0.004 |
| 761.7/678 | 31.403 | 26.199 | 0.047 | 23.715 | 12.486 | 0.000 |
| 523.5/679 | 77.313 | 62.012 | 0.008 | 320.738 | 151.609 | 0.000 |
| 797.7/679 | 291.565 | 236.479 | 0.043 | 1281.437 | 607.982 | 0.000 |
| 823.2/679 | 6.644 | 4.660 | 0.043 | 34.537 | 21.806 | 0.008 |
| 824.2/680 | 6.264 | 4.224 | 0.012 | 19.055 | 11.641 | 0.004 |
| 826.2/680 | 5.944 | 3.691 | 0.001 | 12.524 | 6.447 | 0.001 |
| 829.2/680 | 3.268 | 1.981 | 0.022 | 8.904 | 6.040 | 0.024 |
| 831.3/683 | 2.226 | 1.503 | 0.015 | 9.058 | 3.774 | 0.000 |
| 844.3/684 | 3.969 | 2.506 | 0.008 | 12.483 | 6.862 | 0.003 |
| 836.4/685 | 2.717 | 1.478 | 0.035 | 11.895 | 4.570 | 0.000 |
| 838.2/685 | 4.679 | 3.059 | 0.043 | 14.631 | 6.595 | 0.001 |
| 841.2/685 | 4.335 | 2.313 | 0.040 | 12.465 | 4.792 | 0.003 |
| 828.2/688 | 3.455 | 2.177 | 0.037 | 9.281 | 4.870 | 0.005 |
| 844.5/688 | 6.114 | 4.747 | 0.043 | 11.946 | 7.215 | 0.018 |
| 846.3/688 | 12.634 | 8.818 | 0.002 | 17.950 | 11.332 | 0.008 |
| 847.3/688 | 10.830 | 7.871 | 0.026 | 17.088 | 11.647 | 0.012 |
| 849.5/689 | 15.165 | 11.151 | 0.032 | 21.787 | 12.963 | 0.006 |
| 852.2/689 | 14.537 | 10.814 | 0.049 | 33.914 | 18.303 | 0.001 |
| 553.8/690 | 0.388 | 0.225 | 0.046 | 1.199 | 0.534 | 0.001 |
| 861.2/690 | 6.711 | 4.961 | 0.031 | 15.058 | 7.493 | 0.005 |
| 565.5/693 | 100.772 | 80.472 | 0.004 | 204.541 | 92.075 | 0.000 |
| 566.5/693 | 39.328 | 33.550 | 0.036 | 81.626 | 36.813 | 0.000 |
| 864.8/693 | 3945.021 | 3202.000 | 0.007 | 5929.751 | 3489.109 | 0.000 |
| 865.8/693 | 2079.054 | 1681.559 | 0.008 | 4001.476 | 2100.497 | 0.000 |
| 867.3/695 | 15.922 | 11.511 | 0.022 | 25.184 | 14.119 | 0.003 |
| 875.5/698 | 17.329 | 12.054 | 0.016 | 25.906 | 16.047 | 0.019 |
| 873.8/701 | 77.890 | 65.743 | 0.036 | 99.600 | 60.112 | 0.001 |
| 889.4/702 | 8.571 | 6.363 | 0.046 | 12.340 | 7.861 | 0.012 |
| 902.4/707 | 9.185 | 6.558 | 0.018 | 13.074 | 9.086 | 0.012 |
| 893.5/709 | 9.112 | 7.473 | 0.023 | 9.793 | 5.171 | 0.000 |
| 936.9/721 | 2861.443 | 2538.379 | 0.034 | 4129.703 | 3004.694 | 0.002 |
| 961.5/722 | 9.735 | 8.319 | 0.006 | 14.031 | 9.051 | 0.025 |
| 964.4/722 | 11.570 | 9.613 | 0.002 | 16.950 | 11.860 | 0.002 |
| 962.5/723 | 5.029 | 3.273 | 0.036 | 11.102 | 4.655 | 0.000 |
| 974.2/725 | 7.348 | 4.571 | 0.048 | 9.649 | 4.384 | 0.000 |
| 1010.2/734 | 6.351 | 3.665 | 0.039 | 6.389 | 2.950 | 0.034 |
| 1034.5/736 | 1.885 | 1.174 | 0.030 | 3.263 | 1.339 | 0.002 |
| 1047.1/740 | 7.367 | 3.291 | 0.022 | 8.676 | 3.534 | 0.025 |
| 966/787 | 2.070 | 1.495 | 0.035 | 6.085 | 2.021 | 0.000 |
| 991.1/806 | 2.239 | 1.188 | 0.044 | 6.804 | 1.033 | 0.003 |
| 977/859 | 0.648 | 0.308 | 0.045 | 5.580 | 0.736 | 0.010 |
| 577/908 | 2.999 | 2.475 | 0.010 | 3.001 | 1.743 | 0.004 |
| 653/908 | 5.337 | 4.547 | 0.023 | 5.173 | 2.691 | 0.001 |
| 899/908 | 2.788 | 1.953 | 0.041 | 3.623 | 1.136 | 0.000 |

TABLE 9
Metabolites identified as statistically significantly more abundant in both
rpoB mutant versus wild-type parent strains of MTB, with putative identifications.
RT = retention time; WT = wild-type; PDIM = pthiocerol dimycoserosate; PG = phenolic glycolipid

| | | | | | | | |
|---|---|---|---|---|---|---|---|
| | | Average | Average | | Average | Average | |
| Mass | RT | Haarlem | Haarlem | | Beijing | Beijing | |
| (M+H)+ | (sec) | rpoB | WT | p-value | rpoB | WT | p-value |

Spectra in Chloroform fraction Identified as diacylglycerol
phosphocholine-related polymer* (Score = 3)

| | | | | | | | |
|---|---|---|---|---|---|---|---|
| 200.1 | 318 | 11.598 | 4.620 | 0.043 | 6.580 | 3.204 | 0.001 |
| 215.1 | 318 | 217.697 | 89.613 | 0.050 | 126.643 | 62.609 | 0.001 |
| 747.6 | 318 | 444.030 | 189.220 | 0.048 | 243.566 | 119.076 | 0.001 |
| 819.7 | 318 | 74.700 | 31.109 | 0.048 | 41.029 | 25.357 | 0.015 |
| 838.7 | 318 | 54.247 | 17.966 | 0.046 | 26.170 | 11.222 | 0.001 |
| 780.6 | 487 | 31.377 | 20.849 | 0.044 | 126.434 | 63.609 | 0.032 |
| 807.6 | 517 | 110.520 | 69.352 | 0.041 | 396.873 | 59.912 | 0.000 |
| 808.6 | 517 | 40.775 | 27.568 | 0.049 | 127.011 | 25.237 | 0.000 |
| 864.8 | 693 | 3945.021 | 3202.000 | 0.007 | 5929.751 | 3489.109 | 0.000 |
| 865.8 | 693 | 2079.054 | 1681.559 | 0.008 | 4001.476 | 2100.497 | 0.000 |

Spectra in Chloroform fraction Identified as a PDIM derivative** (Score = 1)

| | | | | | | | |
|---|---|---|---|---|---|---|---|
| 797.7 | 679 | 291.565 | 236.479 | 0.043 | 1281.437 | 607.982 | 0.000 |
| 823.2 | 679 | 6.644 | 4.660 | 0.043 | 34.537 | 21.806 | 0.008 |

Spectra in Methanol fraction Identified as a Hex-N-acetyl-hexosamine-fucose-N-acetyl-hexosamine
related polymer* (Score = 3)

| | | | | | | | |
|---|---|---|---|---|---|---|---|
| 321.2 | 1016 | 60.948 | 46.177 | 0.047 | 42.502 | 29.030 | 0.003 |
| 567.4 | 1016 | 98.960 | 63.557 | 0.025 | 56.887 | 33.203 | 0.014 |
| 636.5 | 1016 | 48.163 | 32.947 | 0.048 | 29.572 | 14.773 | 0.011 |
| 637.5 | 1016 | 16.337 | 7.594 | 0.038 | 9.879 | 3.414 | 0.020 |
| 717.6 | 1016 | 8520.133 | 6005.559 | 0.048 | 5015.322 | 2481.935 | 0.002 |
| 719.6 | 1016 | 631.562 | 470.320 | 0.043 | 439.111 | 292.037 | 0.027 |
| 736.6 | 1016 | 126.813 | 87.716 | 0.017 | 359.462 | 193.015 | 0.007 |
| 754.6 | 1016 | 224.169 | 158.316 | 0.047 | 139.541 | 79.460 | 0.004 |
| 771.6 | 1016 | 408.037 | 265.790 | 0.049 | 226.849 | 117.176 | 0.005 |
| 880.6 | 1016 | 15.607 | 6.143 | 0.024 | 8.211 | 2.613 | 0.005 |

Spectra in Methanol fraction Identified as a PG derivative***(Score = 2)

| | | | | | | | |
|---|---|---|---|---|---|---|---|
| 187.1 | 1061 | 208.350 | 1143.458 | 10.016 | 160.6621 | 110.637 | 0.011 |
| 265.1 | 1061 | 21.793 | 13.452 | 0.028 | 17.680 | 12.084 | 0.027 |
| 345.2 | 1061 | 10.419 | 5.687 | 0.034 | 14.349 | 5.727 | 0.019 |
| 363.2 | 1061 | 74.866 | 49.437 | 0.014 | 63.242 | 44.048 | 0.003 |
| 389.3 | 1061 | 53.262 | 34.879 | 0.024 | 46.866 | 31.877 | 0.017 |
| 531.5 | 1061 | 151.562 | 91.939 | 0.036 | 109.917 | 56.560 | 0.015 |
| 532.5 | 1061 | 56.525 | 36.064 | 0.028 | 42.000 | 20.758 | 0.012 |
| 636.5 | 1061 | 47.548 | 23.712 | 0.004 | 38.509 | 17.748 | 0.009 |
| 699.6 | 1061 | 33.271 | 18.839 | 0.023 | 24.653 | 12.342 | 0.021 |
| 717.6 | 1061 | 6257.378 | 3655.220 | 0.033 | 4529.182 | 1897.650 | 0.007 |
| 718.6 | 1061 | 2867.806 | 1806.767 | 0.045 | 2124.351 | 1002.772 | 0.008 |
| 735.6 | 1061 | 799.634 | 409.858 | 0.034 | 546.444 | 249.317 | 0.014 |
| 736.6 | 1061 | 83.305 | 52.974 | 0.030 | 249.131 | 128.015 | 0.011 |
| 753.6 | 1061 | 170.532 | 104.240 | 0.038 | 123.990 | 62.392 | 0.007 |
| 754.6 | 1061 | 94.545 | 61.712 | 0.036 | 71.979 | 43.733 | 0.031 |
| 771.6 | 1061 | 198.991 | 117.007 | 0.014 | 131.692 | 70.860 | 0.007 |
| 772.6 | 1061 | 107.522 | 67.597 | 0.009 | 80.007 | 42.733 | 0.016 |
| 773.6 | 1061 | 31.654 | 18.005 | 0.015 | 22.130 | 11.537 | 0.025 |
| 789.6 | 1061 | 247.516 | 151.338 | 0.036 | 177.175 | 94.146 | 0.015 |
| 790.6 | 1061 | 124.074 | 79.885 | 0.024 | 91.662 | 53.478 | 0.013 |
| 791.6 | 1061 | 37.056 | 23.549 | 0.046 | 27.228 | 15.857 | 0.012 |
| 807.6 | 1061 | 296.842 | 176.814 | 0.033 | 201.738 | 97.621 | 0.008 |
| 808.6 | 1061 | 166.267 | 93.964 | 0.015 | 109.848 | 52.227 | 0.022 |
| 809.6 | 1061 | 52.777 | 30.453 | 0.030 | 36.652 | 20.813 | 0.017 |

*Identified by analysis in LipidMaps or Massbank
**Identified by comparison of MS spectra produced by analysis of purified PDIM from MTB
***Identified by query of published reports of MTB PG mass spectra
Scoring system based on metabolite identification guidelines (Sumner et al, 2007).
Score = 1: Identified compound. Retention time and mass spectrum of interest matches a purified standard.
Score = 2: Putatively annotated compound. Based on spectral similarity to public spectral libraries or published data.
Score = 3: Putatively characterized compound classes. Based on spectral similarity to known compounds of a chemical class.

Eighty-seven molecular features from the methanol fraction were significantly more abundant (p<0.05) in both resistant strains when compared to their susceptible pairs (Table 8). A large portion of these molecular features were observed in two clusters at a retention time of 16.9 minutes suggesting that they may be derived from a common parent compound (Table 9). Each cluster of peaks was searched against the Massbank spectral database and both returned matches to hexose-N acetyl-hexosamine-fucose-N-acetyl-hexosamine (Table 9). To determine if this compound originated from peptidoglycan, an MTB petidoglycan standard, obtained through the TVTRMC, was analyzed by LC-MS using identical conditions. Comparison of the standard peptidoglycan spectrum did not correlate with the molecular features observed in the methanol sample fraction. However, this was not entirely unexpected given the extensive cross-linking in mature PG and stability of the mature product. An analysis of the literature for mass spectra of partially hydrolyzed PG demonstrated reports of PG MurNac-tetra and penta peptides, and MurNac of Mtb PG that do match the molecular features observed in the spectra from our methanol sample fractions. Specifically, the molecular features at 808.6 m/z (790.6 m/z; minus water), 736.6 m/z (718.6 m/z; minus water), and 531.5 m/z correspond to these three PG derivatives (Lavollay et al., 2008, J Bacteriol 190:4360-6; Mahapatra et al., 2005, J Bacteriol 187:2341-7). An examination of the gene cluster associated with peptidoglycan biosynthesis in Mtb (Rv2158c-2152c) did not however reveal significantly increased spectral counts in the rpoB mutant isolates using GSEA. A previous reported scoring system of 1-4 (1 high, 4 low) was used to determine the level of confidence in metabolite identification.

PDIM Locus Upregulation by rpoB Mutant MTB

It is hypothesized that rpoB mutation would lead to upregulation of MTB genes involved in secondary metabolism, given known relationships between rpoB mutation and upregulation of genes involved in natural product biosynthesis in other organisms (Hosaka et al., 2009, Nat Biotechnol 27:462-4; Hu et al., 2002, J Bacteriol 184:3984-91; Inaoka et al., 2004, J Biol Chem 279:3885-92). This study, which is the first to comprehensively compare the proteomes and metabolomes of rpoB mutant, rifampicin-resistant MTB with those of their paired wild-type, rifampicin susceptible parent strains, demonstrates that several genes, particularly those involved in the biosynthesis of cell wall lipids including PDIM, are upregulated in rpoB mutants, both in broth culture and, in particular, when grown in murine macrophages. Using the program NP.searcher, it was discovered that 4 gene loci possibly related to natural product biosynthesis in MTB, and the GSEA indicated that of these four, the proteins encoded by ppsA-E and drrA were more abundant in the cell wall of both rpoB mutants relative to their wild-type parent strains. Furthermore, ‘in situ’ over-expression of several pps genes were confirmed using a CDC1551 strain as well as a macrophage model for the Beijing isolate, indicating the changes in expression of ppsA-E are unlikely to be due to inherent characteristics of the two clinical strains used or the in vitro culture system. The present study is different from previous studies demonstrating that exposure to rifampicin induces several changes in rifampicin resistant MTB gene expression, including marked up-regulation of drug efflux-pump related genes (Gupta et al., 2010, Microb Drug Resist 16:21-8; Louw et al., 2011, Am J Respir Crit Care Med 184:269-76). Specifically, in this study the impact of rpoB mutations on the proteome and metabolome of rifampicin-resistant and susceptible strains was performed in the absence of rifampicin in order to determine protein and metabolite abundance changes that are associated with rpoB mutations independent of drug exposure.

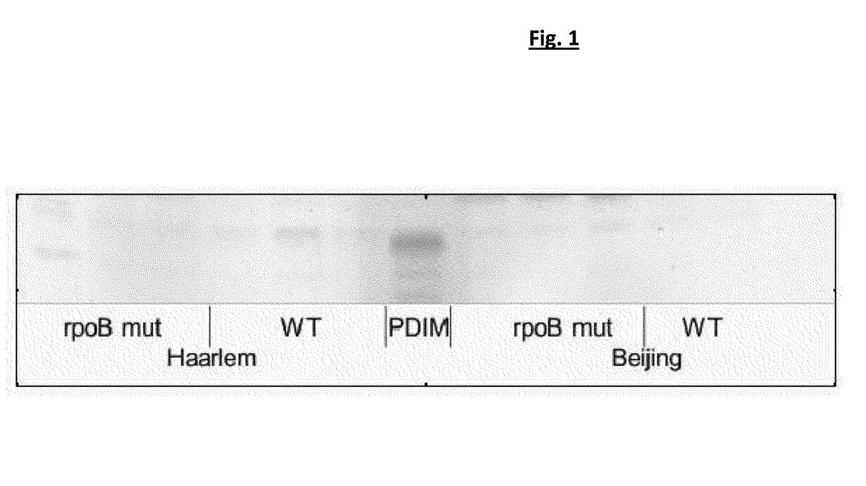

Proteins encoded by ppsA-E are involved in PDIM biosynthesis and the corresponding genes are grouped on a 50-kb fragment of the MTB chromosome (Camacho et al., 2001, J Biol Chem 276:19845-54). Although evidence of higher levels of intact PDIM in the Haarlem rpoB mutant was not observed, levels of PDIM in the Beijing rpoB mutant isolate appear to be elevated when compared to its isogenic strain as evaluated by TLC (FIG. 1). However, metabolites with the same spectra as PDIM precursors were detected at higher levels in both rpoB mutant isolates compared to their wild-type progenitor isolates. While only two strains were evaluated, the finding of remarkably similar changes in protein abundance in a Beijing and Haarlem strain of MTB with different rpoB mutations grown in the absence of drug exposure suggests that rpoB mutation and rifampicin resistance in MTB lead to a pattern of differential protein and metabolite abundance that is conserved across both mutations and strains.

PDIM is a long-chain β-diol (phthiocerol) esterified with two branched-chain mycocerosic acids located in the outer mycobacterial cell wall that has been implicated in MTB virulence (Camacho et al., 1999, Mol Microbiol 34:257-67; Cox et al., 1999, Nature 402:79-83; Reed et al., 2004, Nature 431:84-7). The genes ppsA-E (Rv2931-Rv2935) encode a type I modular polyketide synthase responsible for biosynthesis of the phtiocerol backbone of PDIM, with PpsA-C sequentially loading ketide units onto long chain fatty acids and PpsD and PpsE subsequently extending the phthiocerol further by adding a 4-methyl branch and malonyl- or methylmalonyl-CoA, respectively (Azad et al., 1997, J Biol Chem 272:16741-5; Trivedi et al., 2005, Mol Cell 17:631-43). The genes drrA-C(Rv2936-Rv2938) are located adjacent to ppsE (Rv2935) and are thought to encode proteins involved in the transport and localization of PDIM across the cell membrane (Braibant et al., 2000, FEMS Microbiol Rev 24:449-67; Camacho et al., 2001, J Biol Chem 276:19845-54). Others have demonstrated upregulation of genes in this pathway in response to acid exposure (Golby et al., 2007, Microbiology 153:3323-36; Rustad et al., 2008, PLoS One 3:e1502), hypoxia (Park et al., 2003, Mol Microbiol 48:833-43; Rustad et al., 2008, PLoS One 3:e1502), and treatment with antibiotics such as clofazamine and rifapentine (Boshoff et al., 2004, J Biol Chem 279:40174-84). Furthermore, MTB strains with defects in this pathway have been shown to have increased cell envelope permeability (Camacho et al., 2001, J Biol Chem 276:19845-54) and are more susceptible to IFN-γ mediated and IFN-γ-independent immunity (Kirksey et al., 2011, Infect Immun 79:2829-38; Murry, et al, 2009, J Infect Dis 200:774-82). Importantly, PDIM deficiency appears to be particularly important to MTB growth in the host environment, as isolates with deficiencies in this pathway have pronounced growth defects in the spleens and lungs of infected mice (Kondo and Kanai, 1976, Jpn J Med Sci Biol 29:199-210; Camacho et al., 1999, Mol Microbiol 34:257-67; Cox et al., 1999, Nature 402:79-83) and are more susceptible to the nitric-oxide-dependent killing of macrophages (Rousseau et al., 2004, Cell Microbiol 6:277-87). More recently, PDIM has been shown to play a role in MTB's interaction with the host macrophage cell envelope, inducing changes that favor receptor-mediated phagocytosis of the bacterium (Astarie-Dequeker et al., 2009, PLoS Pathog 5:e1000289). In this study, it was observed that the upregulation of PDIM by rpoB mutants relative to wild-type isolates, initially identified by proteomics experiments on MTB isolates grown in broth, was more dramatic when these comparisons were performed in activated murine macrophages. Thus, although the physiologic importance of PDIM upregulation by rpoB mutants cannot be discerned from these data, it is possible that upregulation of the PDIM pathway is related to an increased pressure for rpoB mutants to maintain or remodel the cell wall, particularly during host cell infection. Of note, certain other bacteria have been shown to increase lipid abundance in association with rpoB mutations, which suggests that this may be a conserved response to development of drug resistance (Vitali et al., 2008, Int J Antimicrob Agents 31:555-60).

The finding of increased peptidoglycan precursors in the rpoB mutants in the metabolomics data is also supportive of this hypothesis. Peptidoglycan biosynthesis is a multi-enzyme process that involves the MurA-G cluster in addition to MurX and a putative flippase (Cole, S. T. 2005. TB and the tubercle bacillus. ASM Press, Washington, D.C.). The instant proteomic analysis identified three of the seven Mur proteins (MurD-F) in the cell wall, membrane and cytosol, which did not show significant differences in their abundance between resistant vs susceptible strains. However, given the lack of data on the relative abundance of the other peptidoglycan biosynthetic enzymes and the complex nature of peptidoglycan precursor biosynthesis, the idea that the increase amount of PG related metabolites could be associated with an increase in the production of PG enzymes cannot be discarded, especially with those involved in the last steps of the biosynthetic pathway, such as MurG and MurX (a.k.a MraY). Nonetheless, peptidoglycan and PDIM are both significant components of the cell envelope, with peptidoglycan forming the inner cell wall layer and PDIM decorating the outer layer via non-covalent association with other cell envelope lipids (Brennan, 2003, Tuberculosis (Edinb) 83:91-7). Peptidoglycan interacts (at least indirectly) with PDIM to form the mature mycobacterial cell envelope, in that PDIIM translocates across peptidoglycan via mmpl7 to reside in the outer layer of the cell envelope (Jain and Cox, 2005, PLoS Pathog 1:e2). Thus, it is plausible that changes in PG ultrastructure combined with changes in PDIM biosynthesis synergize to affect the overall integrity of the mycobacterial cell envelope together in response to rpoB mutation.

rpoB mutant strains of saprophytic environmental mycobacteria have been found to cohabitate with rifamycin-producing organisms on marine sponges (Izumi et al., 2010, FEMS Microbiol Lett 313:33-40), indicating that ancestors of MTB faced rifamycin exposure long before this class of antibiotics was used to treat TB. Rifamycin resistant, rpoB mutant environmental mycobacteria antagonized by antibiotics secreted by surrounding competitor organisms conceivably would have experienced a survival advantage if they were able to maintain the integrity of their cell wall. Thus, it is possible that upregulation of the PDIM biosynthetic pathway in rpoB mutants of MTB, as demonstrated here both in broth and macrophage culture, is a defense mechanism response to competition interference inherited from MTB's environmental ancestors. Although the physiologic relevance of this gene upregulation to the human pathogen MTB is unknown, it may be important to the survival of rifampicin-resistant mycobacteria in the setting of rifamycin or other antibiotic exposure. In particular, experiments can be designed to comprehensively investigate the degree to which various globally heterogenous strains of rpoB-mutant MTB upregulate PDIM, the mechanisms that result in PDIM upregulation, and the effect of rpoB mutation and PDIM upregulation on susceptibility to various stressors, including existing and novel antibiotics used to treat TB.

The MTB genome contains an expansive repertoire of polyketide synthase genes that in related bacteria are involved in the biosynthesis of various secondary metabolites including erythromycin A and rifamycin B (Cole, S et al., 1998, Nature 393:537-44; Gokhale et al., 2007, Nat Prod Rep 24:267-77; Parish, T., and A. Brown. 2009. Mycobacterium: genomics and molecular biology. Caister Academic Press, Norfolk, UK.). The finding of upregulation of several genes encoding polyketide synthases in both rpoB mutants of MTB is of interest in that it suggests that the adaptive response of MTB to rpoB mutation may be analogous to responses observed in related model organisms. In such, rpoB mutations lead to upregulation of otherwise dormant gene clusters resulting in increased abundance of specific secondary metabolites that are absent or minimally present in wild-type strains (Hosaka et al., 2009, Nat Biotechnol 27:462-4; Zazopoulos et al., 2003, Nat Biotechnol 21:187-90). For example, genes encoding protein enzymes involved in actinorhodin (Act) and undecylprodigiosin (Red) biosynthesis are found in Streptomyces lividans, but normally are not expressed by the corresponding paired wild-type isolates from which they arose (Hu et al., 2002, J Bacteriol 184:3984-91). Certain rifampicin-resistant rpoB mutants of S. lividans, however, produce both Act and Red in abundance, a phenomenon which can be reversed by replacing mutant with wild-type rpoB (Hu et al., 2002, J Bacteriol 184:3984-91). Similarly, specific rpoB mutations in Bacillus subtilis lead to dramatic auto-induction of the antibiotic 3,3′-neotrehalosadiame (NTD), which wild-type strains do not produce (Inaoka et al., 2004, J Biol Chem 279:3885-92). Remarkably, while only certain rpoB mutations are associated with gene upregulation in model organisms, the specific mutations most commonly associated with induced gene expression in non-mycobacterial organisms (Carata et al., 2009, Microb Cell Fact 8:18; Hu et al., 2002, J Bacteriol 184:3984-91; Inaoka et al., 2004, J Biol Chem 279:3885-92) align to mutations most commonly found in clinical rifampicin resistant MTB strains (i.e., positions 445 and 450 in Mtb) (Cavusoglu et al., 2002, J Clin Microbiol 40:4435-8; Herrera et al., 2003, Int J Antimicrob Agents 21:403-8; Mani et al., 2001, J Clin Microbiol 39:2987-90; Telenti et al., 1993, Lancet 341:647-50; Yue et al., 2003, J Clin Microbiol 41:2209-12). This suggests that those rpoB mutations that trigger upregulation of genes involved in biosynthesis of secondary metabolites are evolutionarily advantageous.

Camacho et al demonstrated that the PDIM locus is divided into three transcriptional units, one spanning fadD26 to papA5 (which includes the genes ppsA-E and drrA), another containing only the mas gene, and a third including fadD28 and mmpL7 (Camacho et al., 2001, J Biol Chem 276:19845-54). The transcription of all three units appears to be required for the correct biosynthesis and placement of intact PDIM in the cell (Camacho et al., 2001, J Biol Chem 276:19845-54). The genes ppsC, ppsE and drrA are located within the first transcriptional unit and in this study their corresponding PDIM precursor proteins were detected at statistically significantly higher levels in the rpoB mutants relative to their corresponding wild-type parent strains. Although the limited sensitivity of shotgun proteomics approaches hinders the ability to definitively conclude that proteins found in the other two downstream PDIM transcriptional units were not also upregulated, these findings, together with the metabolomics data, suggest that the first transcriptional unit of the PDIM biosynthetic locus, and not the others, may be specifically upregulated in rpoB mutant isolates of MTB. Comprehensive transcriptomics data can be used to confirm this hypothesis. PDIM production is characteristic of pathogenic, but not environmental, mycobacterial strains (Daffe and Laneelle, 1988, J Gen Microbiol 134:2049-55). This indicates that PDIM production is not important for survival of environmental mycobacteria and, to the extent that the responses of MTB to rpoB mutation may have been inherited from MTB's saprophytic environmental ancestors, provides one speculative explanation for the observation that intact PDIM was not found in greater amounts in rpoB mutant isolates in this study.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of assessing microbial infection in a mammal, the method comprising:
a) contacting at least one polypeptide that is differentially expressed in a drug-resistant strain compared to a corresponding drug-susceptible strain with a biological sample from the mammal; and
b) measuring an immune response in the biological sample, wherein an increase in the immune response to the at least one polypeptide that is differentially expressed in a drug-resistant strain compared to a corresponding drug-susceptible strain in the biological sample of the mammal indicates a past or present microbial infection with a drug-resistant strain.

2. The method of claim 1, wherein the at least one polypeptide that is differentially expressed in a drug-resistant strain compared to a corresponding drug-susceptible strain is selected from the group consisting of SEQ ID NOs: 1-98 or a fragment thereof.

3. The method of claim 1, wherein the immune response is at least one selected from the group consisting of a cell-mediated response and a humoral response.

4. The method of claim 1, wherein the biological sample comprises at least one selected from the group consisting of whole blood, serum, plasma, and peripheral blood mononuclear cells.

5. The method of claim 1, wherein the microbial infection is M. tuberculosis infection.

6. The method of claim 3, wherein the cell-mediated response is a T cell response.

7. The method of claim 6, wherein the T cell response is measured by detecting expression of a marker for T cell activation or proliferation.

8. The method of claim 7, wherein the T cell response is measured by detecting secretion of a cytokine.

9. The method of claim 3, wherein the humoral response is measured by
a) contacting the biological sample of the mammal with the at least one polypeptide that is differentially expressed in a drug-resistant strain compared to a corresponding drug-susceptible strain for a sufficient amount of time to allow complex formation between the at least one polypeptide with an antibody present in the biological sample, and
b) detecting a complex formed between an antibody in the biological sample and the antigen, wherein detection of the complex is indicative of infection by the drug-resistant strain in the mammal.

10. The method of claim 1, wherein the mammal is a human.

11. The method of claim 1, wherein the mammal is receiving or has previously received a therapeutic intervention.

12. A kit for use in determining the presence of a drug-resistant strain in a mammal, the kit comprising at least one polypeptide that is differentially expressed in a drug-resistant strain compared to a corresponding drug-susceptible strain.

13. The kit of claim 12, wherein the drug-resistant strain is M. tuberculosis.

14. The kit of claim 12, wherein the at least one polypeptide that is differentially expressed in a drug-resistant strain compared to a corresponding drug-susceptible strain is selected from the group consisting of SEQ ID NOs: 1-98 or a fragment thereof.

15. The kit of claim 12, wherein the at least one polypeptide that is differentially expressed in a drug-resistant strain compared to a corresponding drug-susceptible strain is used to detect an immune response in a biological sample from a mammal.

16. The kit of claim 15, wherein the biological sample comprises at least one selected from the group consisting of whole blood, serum, plasma, and peripheral blood mononuclear cells.

17. The kit of claim 15, wherein the immune response is at least one selected from the group consisting of a cell-mediated response and a humoral response.

18. The kit of claim 17, wherein the cell-mediated response is measured by detecting expression of a marker for T cell activation or proliferation.

19. The kit of claim 17, wherein the cell-mediated response is measured by detecting secretion of a cytokine.

20. The kit of claim 17, wherein the humoral response is detected by detecting a complex formed between an antibody in the biological sample and the at least one polypeptide that is differentially expressed in a drug-resistant strain compared to a corresponding drug-susceptible strain.
